SEQUENCE LISTING (15 sequences)

SEQ ID NO: 1 - DNA, 1458 bp, Homo sapiens
atgtcggtgccgctgctgaagatcggggtcgtgctgagcaccatggccatgatcactaactggatgtcccagacgctgccctcgctggtgggcctcaacaccaccaagctctcggcggccggcggcgggacgctggaccgcagcaccggcgtgctgcccaccaaccctgaggagagctggcaggtgtacagctctgcccaggacagcgagggcaggtgtatctgcacagtggtcgccccacagcagaccatgtgttcacgggatgcccgcacaaaacagctgaggcagctactggagaaggtgcagaacatgtctcaatccatagaggtcttggacaggcggacccagagagacttgcagtacgtggagaagatggagaaccaaatgaaaggactggagtccaagttcaaacaggtggaggagagtcataagcaacacctggccaggcagtttaaggcgataaaagcgaaaatggatgaacttaggcctttgatacctgtgttggaagagtacaaggccgatgccaaattggtattgcagtttaaagaggaggtccagaatctgacgtcagtgcttaacgagctgcaagaggaaattggcgcctatgactacgatgaacttcagagcagagtgtccaatcttgaagaaaggctccgtgcatgcatgcaaaaactagcttgcgggaagttgacgggcatcagtgaccccgtgactgtcaagacctccggctcgaggttcggatcctggatgacagaccctctcgcccctgaaggcgataaccgggtgtggtacatggacggctatcacaacaaccgcttcgtacgtgagtacaagtccatggttgacttcatgaacacggacaatttcacctcccaccgtctcccccacccctggtcgggcacggggcaggtggtctacaacggttctatctacttcaacaagttccagagccacatcatcatcaggtttgacctgaagacagagaccatcctcaagacccgcagcctggactatgccggttacaacaacatgtaccactacgcctggggtggccactcggacatcgacctcatggtggacgagagcgggctgtgggccgtgtacgccaccaaccagaacgctggcaacatcgtggtcagtaggctggaccccgtgtccctgcagaccctgcagacctggaacacgagctaccccaagcgcagcgccggggaggccttcatcatctgcggcacgctgtacgtcaccaacggctactcagggggtaccaaggtccactatgcataccagaccaatgcctccacctatgaatacatcgacatcccattccagaacaaatactcccacatctccatgctggactacaaccccaaggaccgggccctgtatgcctggaacaacggccaccagatcctctacaacgtgaccctcttccacgtcatccgctccgatgagttgtag

SEQ ID NO: 2 - protein, 485 aa, Homo sapiens
MSVPLLKIGVVLSTMAMITNWMSQTLPSLVGLNTTKLSAAGGGTLDRSTGVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKAIKAKMDELRPLIPVLEEYKADAKLVLQFKEEVQNLTSVLNELQEEIGAYDYDELQSRVSNLEERLRACMQKLACGKLTGISDPVTVKTSGSRFGSWMTDPLAPEGDNRVWYMDGYHNNRFVREYKSMVDFMNTDNFTSHRLPHPWSGTGQVVYNGSIYFNKFQSHIIIRFDLKTETILKTRSLDYAGYNNMYHYAWGGHSDIDLMVDESGLWAVYATNQNAGNIVVSRLDPVSLQTLQTWNTSYPKRSAGEAFIICGTLYVTNGYSGGTKVHYAYQTNASTYEYIDIPFQNKYSHISMLDYNPKDRALYAWNNGHQILYNVTLFHVIRSDEL

SEQ ID NO: 3 - DNA, 462 bp, Homo sapiens
atgtcggtgccgctgctgaagatcggggtcgtgctgagcaccatggccatgatcactaactggatgtcccagacgctgccctcgctggtgggcctcaacaccaccaagctctcggcggccggcggcgggacgctggaccgcagcaccggcgtgctgcccaccaaccctgaggagagctggcaggtgtacagctctgcccaggacagcgagggcaggtgtatctgcacagtggtcgccccacagcagaccatgtgttcacgggatgcccgcacaaaacagctgaggcagctactggagaaggtgcagaacatgtctcaatccatagaggtcttggacaggcggacccagagagacttgcagtacgtggagaagatggagaaccaaatgaaaggactggagtccaagttcaaacaggtggaggagagtcataagcaacacctggccaggcagtttaagggctaa

SEQ ID NO: 4 - protein, 153 aa, Homo sapiens
MSVPLLKIGVVLSTMAMITNWMSQTLPSLVGLNTTKLSAAGGGTLDRSTGVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKG

SEQ ID NO: 5 - DNA, 1374 bp, Homo sapiens
atgcacccggcccggaagctcctcagcctcctcttcctcatcctgatgggcactgaactcactcaagtgctgcccaccaaccctgaggagagctggcaggtgtacagctctgcccaggacagcgagggcaggtgtatctgcacagtggtcgccccacagcagaccatgtgttcacgggatgcccgcacaaaacagctgaggcagctactggagaaggtgcagaacatgtctcaatccatagaggtcttggacaggcggacccagagagacttgcagtacgtggagaagatggagaaccaaatgaaaggactggagtccaagttcaaacaggtggaggagagtcataagcaacacctggccaggcagtttaaggcgataaaagcgaaaatggatgaacttaggcctttgatacctgtgttggaagagtacaaggccgatgccaaattggtattgcagtttaaagaggaggtccagaatctgacgtcagtgcttaacgagctgcaagaggaaattggcgcctatgactacgatgaacttcagagcagagtgtccaatcttgaagaaaggctccgtgcatgcatgcaaaaactagcttgcgggaagttgacgggcatcagtgaccccgtgactgtcaagacctccggctcgaggttcggatcctggatgacagaccctctcgcccctgaaggcgataaccgggtgtggtacatggacggctatcacaacaaccgcttcgtacgtgagtacaagtccatggttgacttcatgaacacggacaatttcacctcccaccgtctcccccacccctggtcgggcacggggcaggtggtctacaacggttctatctacttcaacaagttccagagccacatcatcatcaggtttgacctgaagacagagaccatcctcaagacccgcagcctggactatgccggttacaacaacatgtaccactacgcctggggtggccactcggacatcgacctcatggtggacgagagcgggctgtgggccgtgtacgccaccaaccagaacgctggcaacatcgtggtcagtaggctggaccccgtgtccctgcagaccctgcagacctggaacacgagctaccccaagcgcagcgccggggaggccttcatcatctgcggcacgctgtacgtcaccaacggctactcagggggtaccaaggtccactatgcataccagaccaatgcctccacctatgaatacatcgacatcccattccagaacaaatactcccacatctccatgctggactacaaccccaaggaccgggccctgtatgcctggaacaacggccaccagatcctctacaacgtgaccctcttccacgtcatccgctccgatgagttgtag

SEQ ID NO: 6 - protein, 457 aa, Homo sapiens
MHPARKLLSLLFLILMGTELTQVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKAIKAKMDELRPLIPVLEEYKADAKLVLQFKEEVQNLTSVLNELQEEIGAYDYDELQSRVSNLEERLRACMQKLACGKLTGISDPVTVKTSGSRFGSWMTDPLAPEGDNRVWYMDGYHNNRFVREYKSMVDFMNTDNFTSHRLPHPWSGTGQVVYNGSIYFNKFQSHIIIRFDLKTETILKTRSLDYAGYNNMYHYAWGGHSDIDLMVDESGLWAVYATNQNAGNIVVSRLDPVSLQTLQTWNTSYPKRSAGEAFIICGTLYVTNGYSGGTKVHYAYQTNASTYEYIDIPFQNKYSHISMLDYNPKDRALYAWNNGHQILYNVTLFHVIRSDEL

SEQ ID NO: 7 - DNA, 378 bp, Homo sapiens
atgcacccggcccggaagctcctcagcctcctcttcctcatcctgatgggcactgaactcactcaagtgctgcccaccaaccctgaggagagctggcaggtgtacagctctgcccaggacagcgagggcaggtgtatctgcacagtggtcgccccacagcagaccatgtgttcacgggatgcccgcacaaaacagctgaggcagctactggagaaggtgcagaacatgtctcaatccatagaggtcttggacaggcggacccagagagacttgcagtacgtggagaagatggagaaccaaatgaaaggactggagtccaagttcaaacaggtggaggagagtcataagcaacacctggccaggcagtttaagggctaa

SEQ ID NO: 8 - protein, 125 aa, Homo sapiens
MHPARKLLSLLFLILMGTELTQVLPTNPEESWQVYSSAQDSEGRCICTVVAPQQTMCSRDARTKQLRQLLEKVQNMSQSIEVLDRRTQRDLQYVEKMENQMKGLESKFKQVEESHKQHLARQFKG

SEQ ID NO: 9 - DNA, 1680 bp, Homo sapiens
atgccgctagtgaaaagaaacatcgatcctaggcacttgtgccacacagcactgcctagaggcattaagaatgaactggaatgtgtaaccaatatttccttggcaaatataattagacaactaagtagcctaagtaaatatgctgaagatatatttggagaattattcaatgaagcacatagtttttccttcagagtcaactcattgcaagaacgtgtggaccgtttatctgttagtgttacacagcttgatccaaaggaagaagaattgtctttgcaagatataacaatgaggaaagctttccgaagttctacaattcaagaccagcagcttttcgatcgcaagactttgcctattccattacaggagacgtacgatgtttgtgaacagcctccacctctcaatatactcactccttatagagatgatggtaaagaaggtctgaagttttataccaatccttcgtatttctttgatctatggaaagaaaaaatgttgcaagatacagaggataagaggaaggaaaagaggaagcagaagcagaaaaatctagatcgtcctcatgaaccagaaaaagtgccaagagcacctcatgacaggcggcgagaatggcagaagctggcccaaggtccagagctggctgaagatgatgctaatctcttacataagcatattgaagttgctaatggcccagcctctcattttgaaacaagacctcagacatacgtggatcatatggatggatcttactcactttctgccttgccatttagtcagatgagtgagcttctgactagagctgaggaaagggtattagtcagaccacatgaaccacctccacctccaccaatgcatggagcaggagatgcaaaaccgatacccacctgtatcagttctgctacaggtttgatagaaaatcgccctcagtcaccagctacaggcagaacacctgtgtttgtgagccccactcccccacctcctccaccacctcttccatctgccttgtcaacttcctcattaagagcttcaatgacttcaactcctccccctccagtacctcccccacctccacctccagccactgctttgcaagctccagcagtaccaccacctccagctcctcttcagattgcccctggagttcttcacccagctcctcctccaattgcacctcctctagtacagccctctccaccagtagctagagctgccccagtatgtgagactgtaccagttcatccactcccacaaggtgaagttcaggggctgcctccacccccaccaccgcctcctctgcctccacctggcattcgaccatcatcacctgtcacagttacagctcttgctcatcctccctctgggctacatccaactccatctactgccccaggtccccatgttccattaatgcctccatctcctccatcacaagttatacctgcttctgagccaaagcgccatccatcaaccctacctgtaatcagtgatgccaggagtgtgctactggaagcaatacgaaaaggtattcagctacgcaaagtagaagagcagcgtgaacaggaagctaagcatgaacgcattgaaaacgatgttgccaccatcctgtctcgccgtattgctgttgaatatagtgattcggaagatgattcagaatttgatgaagtagattggttggagtaa

SEQ ID NO: 10 - protein, 559 aa, Homo sapiens
MPLVKRNIDPRHLCHTALPRGIKNELECVTNISLANIIRQLSSLSKYAEDIFGELFNEAHSFSFRVNSLQERVDRLSVSVTQLDPKEEELSLQDITMRKAFRSSTIQDQQLFDRKTLPIPLQETYDVCEQPPPLNILTPYRDDGKEGLKFYTNPSYFFDLWKEKMLQDTEDKRKEKRKQKQKNLDRPHEPEKVPRAPHDRRREWQKLAQGPELAEDDANLLHKHIEVANGPASHFETRPQTYVDHMDGSYSLSALPFSQMSELLTRAEERVLVRPHEPPPPPPMHGAGDAKPIPTCISSATGLIENRPQSPATGRTPVFVSPTPPPPPPPLPSALSTSSLRASMTSTPPPPVPPPPPPPATALQAPAVPPPPAPLQIAPGVLHPAPPPIAPPLVQPSPPVARAAPVCETVPVHPLPQGEVQGLPPPPPPPPLPPPGIRPSSPVTVTALAHPPSGLHPTPSTAPGPHVPLMPPSPPSQVIPASEPKRHPSTLPVISDARSVLLEAIRKGIQLRKVEEQREQEAKHERIENDVATILSRRIAVEYSDSEDDSEFDEVDWLE

SEQ ID NO: 11 - DNA, 507 bp, Homo sapiens
atgccgctagtgaaaagaaacatcgatcctaggcacttgtgccacacagcactgcctagaggcattaagaatgaactggaatgtgtaaccaatatttccttggcaaatataattagacaactaagtagcctaagtaaatatgctgaagatatatttggagaattattcaatgaagcacatagtttttccttcagagtcaactcattgcaagaacgtgtggaccgtttatctgttagtgttacacagcttgatccaaaggaagaagaattgtctttgcaagatataacaatgaggaaagctttccgaagttctacaattcaagaccagcagcttttcgatcgcaagactttgcctattccattacaggagacgtacgatgtttgtgaacagcctccacctctcaatatactcactccttatagagatgatggtaaagaaggtctgaagttttataccaatccttcgtatttctttgatctatggaaagaaaaaatgttgcaagataca

SEQ ID NO: 12 - protein, 169 aa, Homo sapiens
MPLVKRNIDPRHLCHTALPRGIKNELECVTNISLANIIRQLSSLSKYAEDIFGELFNEAHSFSFRVNSLQERVDRLSVSVTQLDPKEEELSLQDITMRKAFRSSTIQDQQLFDRKTLPIPLQETYDVCEQPPPLNILTPYRDDGKEGLKFYTNPSYFFDLWKEKMLQDT

SEQ ID NO: 13 - DNA, 180 bp, Homo sapiens
ctgaggcagctactggagaaggtgcagaacatgtctcaatccatagaggtcttggacaggcggacccagagagacttgcagtacgtggagaagatggagaaccaaatgaaaggactggagtccaagttcaaacaggtggaggagagttataagcaacacctggccaggcagtttaagggc

SEQ ID NO: 14 - protein, 60 aa, Homo sapiens
LRQLLEKVQNMSQSIEVLDRRTQRDLQYVEKMENQMKGLESKFKQVEESHKQHLARQFKG

SEQ ID NO: 15 - DNA, 44442 bp, Genomic DNA
atgcacccggcccggaagctcctcagcctcctcttcctcatcctgatgggcactgaactcactcaagtacgtgcatccaacgccattttcctccctgccaggcgcccggcccggcccctcgggagccccacaaagtccgggaacggctctggctccgcgccgccccgcgcccccggcctgggcgcccgaagtgccggggttggggagggggccagggcgctaggctggacctgggtgggagggaggggtgcaggctgaccggagacggcgctcctccagccccggctcagcagagctgacagctgccccctttttcctaggactccgctgcccccgactccctgctgagaagttcaaagggcagcacgagggggtctttggctgctattgtcatctggagggggaagagtgagagccggatagccaagaccccaggcattttcagaggtggcgggaccttagtcctacccccaacacacacccctgagtggctcatcctccctttgggcataacgctgcccttgggggctccagaaacaggcggtggggattgtgccgctgagacctggaagggcagccagcgtgccggccagctgtgtgcattgctggcttaatatgcagggcttggggggctgtggccacatgcccggcaggaggtgagtgaggagccctgtggcgtgctggtgtggggatcgtgggcatttcaaacgggcttgtcgtaccctgaacaatgtatcaatagagaaaggtctctgcttggtattctccattttaaagatgcatattggagctggcaggtcttgggggaggagaagggctgtctgtgagcgccgaactgggagggttgctttggcactatggtgctcggaagagcctgccagccgagggagccgggctgcttgggagtgacattaaaatgccctttcaatgatgccactgtgccacgctctgaactgggagactctggccctttggagttgcaagtccaggaagtgatgggcagccagtaccttggccccaagccccagctgcccctgacccttctttccttcctcccttcctccatctccctcagaataaaagagaaaacaaagcagagaagatgggagggccagagagcgagaggaagaccacaggagagaagacactgaacgagcttcccttgttttgcctggaagcccacgctggctccctggctctgcccaggatgtgcagtccaaatcccaatccagcagtggggttatgtcgtcccgcttaccctcagagcccttctcctggtgctgcccagacgatcagccagtccctcctggagaggttctgcatggcctctaggagaggttttcttggccccaggaaggcctggtggagggtggtggttgtgcactgttgctggacagatgcattcattcatgtgcacacacacacacacacatgcacacacaggggagcagatacctgcagagaagagccaaccaggtcctgattagtggcaagctgccccacaaagggctatgcctgtgtcttattgagacaccttggcaaagagatggctgattctgggtggtcctggacatggccgcacccaagggccctccaagccttaatggcaccctgaagcctccatgcccaggccaaaagatgcttttcctccctaagttctcctctttgtgtctttttaaagattccctgttctggggagaaacttttggttcaacttcaattagaagccttgtctcagtggtgaaagtatttgtccatccacacgaagggtgctggacatccgaaccccagccagcccctggtcacacccctgcatcctcacaccttagagtcagggcccagccagccaccttcaggaccctggtcagctccgccagcccacagcctccgcgggtcaggaagggaaatgctgctgttttctttgctggcctggcatgttcctctctctgaagctgaaccacaggctgtctcaggggaatgtgtcccttgactcagccaggaggcacctcccaccctcatggtacagctccaccttccccaggctggcttctgataattgtgctttcggaatcttgcattggaggccagttatcctaatgtgttgggattttggaaaaaagcctcagcaggtgaaatcacgtcaacgtttctgagtctctgaagggggcagagagtggctggctcagcaccagcacccgagagcctggctaccctagtccccacctgggtgggccccactggggttatgccaggtgactttcatctttgacctctaagatggcatctggggtcgggaatgggtgtgggggcaggaccagcctgctctgattccaagagctactgggggacatccatccccaagcatcttactttcttgattccaaagttatgcgttgcgggttccctgagcaatgctgctcatatttgtgagtgagtgaagaagcatgtctgggcttcaggatagggtgctggagctgcctggtgtctgcctcccgcttccactgtaggtaaattgctccaacagccacatccttgcttttagctccttccaagggatggacatgcgattctagggccactgtgtttctaaatgagcatacgtcgacataagaagatgtcagaaacgcctggatcctacctgcacttaagaaatgctgcctggaggattcaggcggacaggcagggggagtggagcacatggcggggccatctgttctcccgacagcttacttacgatgagacactcaatcaaacaccaaccgttgatggatggcctacgacacgcaaggctgtggggccaggtgaactttgtcctcagacagaaagacatttcacatggtcttgctctgtgattttcaacaaaagactggtatttcatgctcttagagaagcagggtaaagtggcatggtactgagcttccggggcagggtccagaggcattgatctctgggccttggggaaggtggggcagggcagggactccctgggctccaggcccaccgccacaccccccaggcttcccttcagagtgaagaatgttttttgtacgtgatagggtaaaaggggctgggaagctttgtagcaacaaccttgaacccagatggcctcattctcaccccgatcccaccagacactggctgtgtgaccttgagaaaggccctttaacccctctgagcctgaccttccttgtttcacaaggaggattaaacaagatgattcttgagtaaaatcccttcctgctacagacccccaaccccattctttccttggctcactcatcctctccttagacaagcatttcctgggtaccagctggttagatttaactcggtcaattctgggcttacaatgttaagtgagtccaaacccttgccgcttggcacctggggccggcttgctttgagggaagaccccaggctctgccataatcatgtgtttcagtggcaaaagaagtgtctgcaccgagtgctctgggtaggagttggggagtgagttctgttttggaagctggaggcagctctggagaaggaggatatttgatctgggtccaggcttgcacctgggtccactagtgagcccgggggaaagaagaagaggacagaggctgggtacggtggctcatacctttaaggccagcactttgatcacttgaggtcaggagttcaaaaccagcccggccaacatgaagaaaccccatttctactaaaaatacaaaaaatgagcagggcgtggtggcagacgcctataatcccagctactcaggagactgagggaggagaatcacttgaacccaggaggcagaggttgcagtgagccgagactgcaccactgcactccagcctgggcgacagagcgagattctgtctcaaaaaaaagaagaagaagaggacagaaatggctgcagacagaggctgcagcaggaagaagggcctggaaaccctgactctcacatccaatgctcaatccacgtgggttgagggtatttgaagtgtttgaagatggaagcggagctctcctcattagccacgcatccccaggccgggcctggcctttcctaaaacatgacacccctggatgcccctggtggggttcaagctgtcagtgaacacagaacaggcttgagtggcaaccgtcactgtgactgtttgccttcaggttttattgagtgactactgtttgctgggtctcggagttgggccagctgtttgccagtctaggtgcctgcattgagaagtgggcagacccgggactgcacttggggagttgtgggttggtgtgaacagggcagacgagaagaccagggaggtgatgctgacttaggctttcagagaaccccagtgaggcccatccctctgaatctgttcctcatccccccatttagctcattctagagctgaagacctcacttcactgccctgagcaaatccaacaagtagaaacggctaaacacttttacctgctgcaacctaagcttgggccaggtggtctggatgagctgtcttgtccccctgccaggccctcttcccctccagcttctctgccccctctgttttggacttgctgggaggatacagtgtttgtagaaggggatggagtgcacgggtagggggaagagttcaggtgaaattggggtcttttcctaaacccattatttcagaatacgtggaattcattcagtctttgcaccaaagttggctgtggcctctcaggctggcaatgcctctggacacacggagggagaggggcaccctcccctttcccattctcccgggctgcctcccctggtggagaggctcctgacaccaggggcgctgagctgtcaatcctgcccatgagacagctgctccgtctcctaaagcaatttgcttctcattccactgacttcaaccttccccaatcagaagaaaggtaatttcctgccttgggttgtatttattactaaagttatcaggccctgtaatcagtattaacatcaccgtgtaaagtaatacaaaactaattactagctaaactgaattagatacatggcaaccacgagctaggctgacagggcgagcagccaatttccagctctgaaagattcgtcctggacaccctgcacgctcggaaacctcagcgctgtcccgactggcaccgcagggcgaccgaagggaggggaagagagaacaggaagtaaaaatgcacacctctgagtttttttagattattaaaataataatctaaatattataaataataatgatagtaaataatatactatataagataaataatgtaagaaactggagcccgcccggagttaaagccccaggaatcccgccagtggcgaggactgataccgcatgtcacgagtgtaaactttatcatgttgcggcggtcccagcaatcctgggcagttgtccccattcttgacctgaggacacacaggcgtggggaggttaagcagcctgcccatccacccagcacatatgtgatgaccccagatggtgctctaggtctgtctgacatctgagaaacctaagttctggtgcttgttaagacagaaagaaaatgcaaagtcgaaaaacagcgcagtgtcaccgaacaccagtgcaaaggaggtgctggtgctgctgccgggtcgccgacctctgtggaatgcaccacagagcccatcagctgtgcctttccgactgtgtggacacgtgcacggccggctgctgaccttgaaaacgggcttttgaaggagaaagcagggcaagaaggagggggtccgcttgtggtttccttgcaacccctgaggatcaaagaccccagcaggcccgtccaccccttggaaacggggatgcatctaggagtgcctgccccgctgcctcagttctctggagtgtaaggttaggggttacgtaggcagccctgtgcccgttaaatgtctcagcatgtgcatgacaacttgagatccaagaagtcagaattaggggccaggcgcactggctcatgcctgtgatcccagcactttgagaggccgaggcaggtggatcacttgaggtcaggagttcgaggccaagaagtaagaaataggatctacaggtcctgagctaaggtggcctctcagtgaggctctaaactgcccgacacgtgcacccgacacgcctgcgggtccggtgaccccctttgttgcacctggcatgctttgcacaaggtggctggaggggctgccatggagagaatccgctgcgttcgaactgacccgtcctcgtgtgtggctgctcacctggagaaggtttcctgtccctgcttcttctgtgctcgtgtcaaagctcacgcccctgcctgggctccagctggtgatggcagctgcagtgttagcagacctgctcgccccaaaaggacatcccagcagcagggatcttgcccgtatattccttatgcatggttcaatgacagttaaaatctgattggagagtgtgtagccatgaaaagttgagatgaaagagtgtaagggggagatgggaatgaggtcggctccccagcccttgtccacattcagatcagcagttcacagcatcgctttgggccgtccctaaagtggttccattccagctagtggttttaggtatacaagttgtcttcggggagtttaaaccctgccgcggggatttccctaccatacaaaatggctctcctgagtggtgctgaaaagaaagtctcccccacttgcttctggtgatgcttcgggggcctaagagtcaaagaaagaacatcagtgtattgtaaataactttactccatgcactgaaggatgtggtttttgacagtgcagtgggactcagaaatggggagccagccatttaccaacaatttggctgtcaacttgagtccttgaacccctgcccagcctccttcccagccccaaatatgcacacacaggtttccgttcccaggccactcagcctcagcatcttgtcccacagatgcacagactgagtcagctctgagtggcagcaggtgaggggtgttgacggacccaggggtgctttgttccatcagcaatacaaaggatgatgaaacaggagattctcccaggggctgcagcagtcttggtgagaggtggctaagaacgtttccgaggccaagtccaggctccacagggcaggccccatcagtaatgcccgggagcctcctgtgcagaggccgggtgtcgtctggatgcaaagaactttgtttggaatctggcagcaccaaatccttggctgtaagcaaatggtgccacctggggcattttccacattcccagctggaccctgggattgactctttgatgtttctcatcatgatgtcacctttgaattcatgtcaggatcaatgactgattccacagaaaccgggctgcctttcccaattagttacggtgagtttcctcacctgggaagtaggaaatgattatgtttgtggggtccctgaaaaacgtgcattcagcccagagggtgacgtgtctgctgaattgtcttgtccctgttcttcaaagtaccactcccagaattggcctctttttccattcggcaactaagggaatacagagctgtggcttcccttggcctggctgtggccaggctgagacgatatccatgaaagaggcgtggaacctgccagggagagtgcggagggtgcatgaagggtctggggggatgggggccgtggacaagtcagctctctggatggacaaggaactaggactgaagagctggacacgaggccaactgctgaatggaggaaacgtcccccagcaacagccatgggtctgtgggatgctctccccagggcaagtagctccccatcactgtcactgggggacttgaagagctgacactgagggaaagggggaaggacgatacctcggaggaagttgaatctcctgggtcggcagctcaggccagccgtgctcacaaaccccaagcacagtggctcatgatggcaacggtgtgcatagggcacaggctgcctgggggcacctctggctcacatgtcctgtcattcgagggccctgggcaagggaggggccctctctggatctgttttcctcatgacagaggcagagagacagggtgtgactgagacatttggctctgaaagctgctcagtcgtggcccttagccgttctccctcccggggccaaaatcgcaggtcacacagccacgcctgatgcaggtggtgtttgggagactgtcagcttatgggaggtgctgccaggcacagggagccgggccaggatggaaatcctctccagggaagggggtgaggagcagttacgcctccacccatgtgtgaacccagaggcctcagaggaggggagtacagggaacacttggctcagaagacccatgctggtcttctgtggctgccgagagggagtgcccaggggacctgtggctcctcccattctgcctggcttgaggccaggtcctttccttgctcctggaatgttccagcacagaaagggaatgaaaaagctacatacaacgttagaaaaaggagaggtggctccatgtgggacctctctctgtgtctcttctctctctgtgtcactctatctttcttctctctctctcttctctctctgtctctcatctctctctctgtctctcctctctccgtctcttctctctctcctctctgtctctattatctctcctctctgtctctcttctgtctctcctctctgtctctattatctctcctctctctgtctctcttctctctgtctctcctctctctgtttctcctctcttctcctctctctctgtctctctctccatttctttgtcttcctctctgtctcctctctgtttctttgtctttctgtctctctgcctttgtctctctctctctctcttcttctccccttctccctccctttcttcctcccctccccttgtttcccttcaagatatttgccaatagcctgaggagagtatgcgtatttttaaacagcaaccgggcatccaaactttgtcccttgagggcatcctttgccaaggagcatcgggaagtggcccagagacctgcttccctccaagcagccactcctggctctgggacctgagtaggtttgcatcctgcggacctcactgttctgactgtgggtgtcagtggtcgtctgactgtgggtgttggtgtcgtctgtgccctatcagctcaccatgtggctgtcctagggcttctcgaggtgattccccaatagcccacgtgtgtccgcctcttgtaaggtctatagcaagctgggcaggagcaccctctcacacttgcgtcttctgagctctgaagccgggagatgcagagggggtgtgcttgtttttatgtgtgttgatgaatttgtacagagtccaccaaggtcaaggcctgtagggggctggcatgaggatagagacgtggctgttctaacctgtcatgacagggagggtggtgcaggctcgtgccaggactcagaaaggggtgggcaacctcctgtccttcccacgagcgtggaaggcaagggcccatgaaatgtcttctggcggcctctgtggctcgtggaactgtgcggacagccagggctgctgtggccatgcaactcatgccacttctggggctcgtgagatttaactatggccatgacatttataagacgaggatgctaccatgtgtatgggcttagccctggaaataggctgtcaccatatgaccttggacaggtcttatcatctttctaggcctgagattcctcatctatacagtggagtcaatgccaccacaccatgcacttcagtaaggattgggtgccaggcatggcaaagccccagtgccaccaggggcgcagcacctgccccatcagcatccgtatgattgttcagagctgcaggtgtgatgagccctggctttaagagaagtcgtgaagaaattgggattgaaagtcctctttaggaacacttggtattgcctggcgccatggatcccctaatccaaatgccttcattcttcaacgatccatactttccttctggaatgctccatggccttctaaatccaattacacttactcaagccttatctgacgaccctttcaaaaccattatgcccactgaatcacagattttagaaactggacaggacagtttggagcaggtgcaggttggagccgctcaggtgcaggttggagccgctcaggtgcaggttagagcagtccaagtgcaggttaatcttttacttcgttaaataaataaatgtctatggcttctgagatggcctgacagttttgtggattggattgggttgcatttgaatttgttcccgcagaacaagtggtccttgtccttccgcagcgggaagcggcgtgagtgatctggacagacacggcttgtggccttgaatcggtgttaaacatgcatggccagaggaggggggcgaagccagcccaaccggacttgtgtctccgcctgggcccagtctgtgagccgggcctgcagtcccagcttacactgggagatggcgcccttccccaacagttggaatttcctggcatccgacccagcccggctgcctgagattacagcattaatcagaaaagcagatctgaggggctcatttaactagcgggtctcacacccagcactcaggccaggatcatcttggctgcagctgaagtctcttcagccgaggactgcgcacacagagagaaaagccccaggaaggcgttgttcctctcaggcgggcgccagggaggcgcgctccttccggcccggcgtccgtctttcaaatcccatccaggaagggagattaattttcgcccaggcagagaagagtgtagtgagtgatctggaggattctttccttcccaaatggctgggaaagcttaatggaaggccccggaggaagtggctttcatctccgattagaagccttctgtaaatgcaaaagccctattaacgtgtttgacccagtcaggcctgcgcttcgggtgggactgacacgcgtgagtcctgctgcggtcgccgcagagggccgggaagaggggcagcgtgcgccacttgccctgcctctgtgcctgggcgccatgactcgagcgccacccctgagtcagtaaggacaccccccaacccacacccctccaccacagaccccagtccccgccccacacacccacctccccaacaccaggttcatgggcgtggtcccttcagctcctgaggtccaggcctgagccccagaccttatgcagctcctgccgggtgtgcgccctcccagggccctcactgcgcaccgcgggccacggcagaccaccccagccccagccttgctgtgcaggtgtcaggagtgccggttggctcccttcctcccaagcaaggccttagggcaccgcggctgccctgggatcgcaggggcgcctttagctctccaccgatgccccgacgcccccctggcgctggaggccctcgcgagtctggctgcttttcggagcctgccctgcctgctgggtttcaggcgacggcccaggctggctgggaccctcgaatcaccgcggaaaagggctccagtaggcaggacggcgccgtctctctgccggcaacctttgccccaaagcggaccctctgcggggatcggagagggatgccccggcgtgaggatgggagaagccccgggacgggagggccgcgggccgtgcccccagctggagtccccgcgccgccgccgggtattttatgatctgggggtggtggtgtgtccgtctcctcatgtcaccctgatcccaactcctgggcggactggagtttgcagacctcgctgccagcagccagggggcggcggggagccgagcgagaggaaaaatccacccatttcctgggcggattgcgtcggtcccgcccggccgagccccgcctcccggccgcggcccccgcgcgcagcccgcgcagcgctcagagccggacggcgcttcccggtggcggcggaggagcccggagggacgcagccgggcaaggcagggcgcagggcgggcggcgcgaggcgcagggcgcggcgggcagaggccacctggccaccttccctggcgcccggggaaggcgcggcgatggccggggcgcgcggggcggcggcggcggcgggcgggcggcggcgggcgagggggcgcggggacacagccaggcgcccctgcccgcccggtgcccgccgctgaaggccgcctgggcgcgggagccggtgccagctcggagcgggcgctggaggcagctcgaggcgcgatgtcggtgccgctgctcaagatcggggtcgtgctgagcaccatggccatgatcactaactggatgtcccagacgctgccctcgctggtgggcctcaacaccaccaagctctcggcggccggcggcgggacgctggaccgcagcaccggcgtaagtgcgcccgccggccgccttggcgcggctcctcctcctcctcctcctccccctcctcggtccggagccccgggctgggcgggcgccgcgcgggacccgagtcgcccagggaggcggcggggagcagggcgggcaagggcaggcgtcgcgggccggcgcagcggtggcgaccctgctccccgctcccccagcctgggccactccatctccgcccgcgcgcccctggggcggcgtttccttcgtctgggcccctcgccgcggggccgggggagcttggtgggttctcggaggcttggagtcctgggtcagtaatcatgagccccccattgaaaaggttaggaaactaaggctggggacttggggacttgtccaaggtcacactcagcgagtgaggggtggagccgccgctagaccctagtctgggctcggtccagcggggactgagcccgccctagtttgtaaaagccagactggccggaccgggctgggagtggggccccagccggtgggctccggagctcctgcccgcgcctgcattcccaaagtcccaaggcgccctttcctccccagtccataggagggtttgttccttctcctccgaggacggtgccgaggggcttgggtggggcccctgggagcctgcccttgggcgctcaccccctcgcctttgccttcgtcttctgcgcgcacccctccctcctggcctctgaaattgaaatcgcgtctccctctcgagcctagcgggaggggaaccgtggccggggctgcttctgggcagagctgacttagatggctgagcgaggctgagctgaaaccgccacccggagggccgcgcggggaaggggccgctgccgggaaggcgcgccccagaccactggccctttaggctgaaaggagaggtgaaggacgtgagtctccctccctctctctttttgctgccaggggtttagtgccctgcgaagagggctccgtggtgtggctccctcctaagacccctcttgaggccgccctccctgctccttcaggaatcggagggcgattcctccatgatgactttgttccggcctgccggtcccgatcttctggggttgggaatgaatgagattaaagatatgactactatagattctacccgcccaacttctcctccccatcttcatgctaaaaatagcagagagatgacctgatgccaccggaggggagcgtgctcagagacagtagggccagcagcaggatggagctgtgtaggctcaggagtgcccgccccggtacctggcagggctgggctgcaaggttagaccagacatgggcagctccggtttctttggtgatctttgggcaaacctcaggactgaggttgggggtgacccctgcacagcatgtcaccagcaatttggcgacctccaccaaatggctcgtggggggagcccacaaggagctggaaacctagtgttttctcgggtcggtctgtgccacgctgccggctgggaggttgctgtagctcggggttcatcacagctctgggcgaataggctgctggatctggggctgttacagtgggtgcttcgtctgcaagtgtcagggaacggcagggcgaggcttggtggtgccagccacccgtgcttgacttacgggaaggatatcctgtttctgaaatacgtgcccgctgcaagccacccagcgtttgctttcctgccttgctcctagtccagcctgtggcctccagttgcccctcccatccacccagcagatcaatttggccagacgacaaactccagacatcccttgagttgaacttaaattttaagaacgtaaagggtcttgcctctttgacatccttccagccctgagccccctaacaggagacctgtgcctctttgttcccagggaccctttcttccctttgggtcaaacatgtcttgggaccatttcctcccatcttttccttgggggtggatgtggacagtttcccctggttgttggctctgatacacaacctcatggtctccctttctctccttccctctcccttcccgccccgcccctctccaggtgctgcccaccaaccctgaggagagctggcaggtgtacagctctgcccaggacagcgagggcaggtgtatctgcacagtggtcgctccacagcagaccatgtgttcacgggatgcccgcacaaaacagctgaggcagctactggagaaggtgagtctgcgcagagtgtgtgagtttgtatgtgtgtgtgtgtgtttgtgtgtgtgtgtgtgtgtacatgcctgtgtgctcacaccagcaccaaggcttggctagcttgcaggccccattttgactctttcctggtttgtctccactcaaaatatttgtgaatgagtgaaagggtggatggacggatgggtggatgggtgggtggatggatggacggatgggtggatgggtgggtggacacacaggtggattgagacttccaagggtggtccaggaagagaggactagacctgccccctgtcaccacaggctccactgagaagtcacatgggcatggtgagacgggacacaaggctcgtctctgagtgatgtcataatctgtgtgcacagggctggggctgtcacccagcagctaccttggagagcgcccgtgggcccaggaatgcatggccatttggattttacgagatgagatcttttttctgcccttgaatggttgtacctggccagtggctggacaggtgctcatcaccatgaatgccttcctggggctaaagcaggccatgctgtcaccttcagaaccacatgggaacagcaggtttgcatatgtcgtttgcataacatattttgaatccgttatttcacttaatcttcacaagaaccatgtgaagttggtgagatttttcactccagttcaagaaaaggaaatttggctcccaaggggtggacttatttgtcccaagccaaacagctgttagtagggaagtgggcactcggacccacatggccgagctcatcacctggttcagtctcctccactccccactttagtcagggggccttctcctaatgggcacagcccaccagccttttggcacattccacccaaggaccccgtattctgtggttaacctgggttctgggggccccataggagagttaccaagggaacaaggaggaatggtgggcccttctgttccacccattgattccccagaagaaaggtgcatttgcatgtagtgcctatgggcagggaactttcccaccctgagggagtggggtcacaccacacacacgcacactcacaaacacactcacacatagtcacacacacactcacacacagtcacacacattcacacatagtcacacacacattcacacagtctcacacacacatagtcacacagtcacactcacatagtcacacactcatagtcacacacacacagatcacacactcacagtcacacactcacacatagtcacacagtcacacacactcacatagtcacacacactcacatagtcacacacacagtcacacacactcacacatagtctcacacacacacagtcacactcacacatagtcacacactcatagtcacacacacccacacatagtcacacacactcacacagtcacacacacactcacacatagtcacacagtcacacacactcacatagtcacacacactcacagtcacacacacactcacacatagtcacacacactcacacacagtcacacacacactcacacatagtcacacacacacacacatagtcacacacgttcacacacacacaatagtaaagccgtgactcttcctgcaggttggccttggactttctggaatgggcgtcagctcaggagtatggcaggaggcctgtgaggggcggggggcctctgtgactcagggctggctccgtgctggggggacagatggtgctgtcactgcccacctcgttggggaaggtgggagcagcctggtgagaggacagcattgacccgactcgggtccacattcctggtgctgaaccacaaaccctcaggaagctcgagacaaagcaagtccccttccttggagaaggagagggtgggctagaggtttcgagtgcccacccagctctgacgggccttgaaaggtccgagtcactgcctgttctgtgtcccagtccctgtctggaatggccacaggacccttgtttcttgggcaagaaggatgcatcatggtacgcagggcaactggcctagttcgtaaagcacgctcaccttgaaggaatagaatttgttttttctttagccacctgagtaaatcattttaaaagaattaaagtaagatacatttaacattccaccaacatttattaacaccaaactagaccaaagcctaactgagcctttaattattcagaagcttagatgggggaggcggcacttggaggggcctctgggctgtgggggccggcgagagtcaggaggggaaaacagagccctgagcccagggaggaggcatttgcggggagagcagagagttgggaaatggattccagaatgctccatgagcccctgggactgcaggccctggaatgttccttcctgcctgtgcgtgccacagcattactgcttaaaaatttaaagccccatttgataccatagcgggcactctattttcagagggcatgagccatttgctatgaaagagggcccttgctccttaatgcactgtgcattttagcaggaggcagcgctgctccgtcctcagcttcccccgcacaccctggcctgtgtgctattttctttctttcctctttctcttttcttctcttgctttctcttcatgggcagaagcaggacagggtgctccagaaacctcagcccatgtgccctgttgatagggctgggtcacagctgagcatattccagccaggtcggtatttctgtgccctggcttcacctttaacccacctggtgaggctgcaaggtactgagtcccaggccccacctccagagtctctgatttctgttgccctggggtccctgtggggaacccaggtatgtacagccctggaagcctcaccaggtagttccatgcagctagggtcgagcactgtagggacacagatagtggcaggcgtgtggcacgggctccgttctctttgaagaagcacttggctctgggtttgagagtgtaggctttggagccagtccagcttgaactggcaggcctcctggctgggtgacagtaagcaagttgcttaacttctctgagccttggtctccctatgtgtaaaatgggactcaaaatagtacttattcatttcccggggttttgtaaagatcaaatgagataaataacacagcagactcagtgcagtgcctggaacatgggaaaggatggataattattagctgttagttatcttgaggccaggtgtgtctttgtgtgttgctggcccccctcccgcttcttaatgcctgctgccccgttgggtagggtgtgggatagactgtcatcccgtagctctgtgaccctgagcctgccagttggctcctcagagacacatcctgcaggcaagaaaatgaaggtgctcagaggaagggctgtgctcagaggaatgccttccctagggttgggagatgggtgcgtgattgcagtcctagactgactgtgagctggaggcagacctgtgcctgtgccttgtccatggccagaaaggaaagtgcattcagggttaggagaactataggagagtaattgtttggttttaaggcagctgaatagcttggtatctttaaaatttgttttttaaatctaaagtctaatctttgaaatcttttaaaagcattccttctctggggagcaaaaagcctacagcaccgtgtgtggtctcctgactgaggccagcatgagctcagcagcttctcctcggcacttgtgggccttcgctcctggggattttgccaggaggggaatggagagaggggtctggactggcgccgccatttgtattgttgcttcctccggccatgtggctgcaggagctactcccgtgcactagatttactggactttgtgcaggggttacagatgtggatgagacccagtgtcactccccccatgagcatcctgtccggaaagctgtggggggacagctgtgtggctcactcctaggcagacattgtgccaacaaagtgacgttgttgtttccaaatagtttgcagctagttttaactcagtccatcagagaagcctccctggaggaggtggcatttgggctgggccttgaaggacgaatatagacaaactttacatcttttcttgaaaaaggtgctgtagttattcaaagtgacagaggaagacaacaagagaatcaggcaggtggtattagaggtatgcttccgtttctcggggaagagaaggggtgagagggagctgaagccaccccgtcgtgtactttacaaacttaaaatttcaagatgagtttgtgtgtgttactttctgataacaagatatgaaattccttgcattttggggtgctgagattggcaatagtggttttccttctactgaggaggcttggtttgctctcttgtattttactttatttggcggggtggaggatgtatagtaagaagtagaaaccacactgtcctcttttcacactgaccggcaaagctaagcctcatccccaaatgattttgttcctagaagggagagtattttcaaattcagcagaaaagtcaggagtgaagatttgctgtcatggaggtaacttcctctctagtaattggattagattctgagaaaatgacccaaacaccactgcagcaatcctcagcttcctcccgcctccccgcagccccgaatgcatttgcacagaagcacacaccagtttcctgcaaataaaatgcactggcaggtggctgcactaaaactgtttttcttttaagctccctggaatcctgttgaatattaaagttccatcttgaggctagactcaattcagatctgcctgtaaagatgtaaaacagcgattcttcatttgctgggtgattgattcctgactctatgctcagaatgtacatgtttgcagagcagtcatctacctatgatactgtggggtgtgatggatggcatagcagatagggaaaaaagcttgatgcagagtcagggagagcagctcccttgttccttcactctgcacttaagtaatcaaccttatcagcaagacagtcaatgagatttaggaagaaggagaatttatacctgggggcggggcagcctaagagtgccattcctctctgctggtgtttatgcttgcctaactaatgttgtcctgcaacagaaagactaggaacaatccccacccaccactaccaaaaaaaaaaaaaaaagagagagagagagatcaagagaaatcacccagcctgtgcctggagctacagcgaataacggaacttgagtctcctacacccctgatttgcatccctgatgataagaacaggttggcaaagaaaatgtttacccaaccaatttgctgtgtttggggagttatcagtcctcacagtccgcgatgctggcgattaccttgtaaataatgcatgggccacttctggttcccagtgtgctggctgtgtgggaaagggcaatgtctgtacgagcaggcagagaagattgcctggcacctactgcggctgttttgctgaacctgttgcccttttgacaggtgcagaacatgtctcaatccatagaggtcttggacaggcggacccagagagacttgcagtacgtggagaagatggagaaccaaatgaaaggactggagtccaagttcaaacaggtggaggagagtcataagcaacacctggccaggcagtttaaggtatgcatgttcctccccctctccctccccttatcctcctcctcctcctcttcctcctccttcccctccctcctctcctccttctcttcttcctcctcctcttctcctcctcttgctcctcctcccttcatcctcctcttcctcctccctctccttcttctcctcctcttcctcctcctccccctcttcatcctcctcttcctcctccctctccttcttctcctcctcttcctcctccctctccttcttctcctcctcttccccctcatcctcttcatcctccttttcctcctccctctccttcttctcctcctcttcctcctccctctccttcttctcctcctctgaggctgggtgtgctttcccttcatgctctccctttccctacagaaatggtcatttggggcagggagaagcatagcaaaggttgttctgtgccttgaaaggactgttgcccttggcagtagggaggccaccactggccctggcttggcagaagccaccttgacaggggcggcctgagtggtggcagcagcatacactcgcccccaagcccccgtcagtgtggtttggaagccaggggtctgagatcctgcgctgcccgagccaagtcgaatattagctgggaagggacatcgttattggcccttgtcattctgcagctgctgcaggtaaatcacattagccaaagattagctgaattgatgagggccattctggagcaggaatctctcagggcagttttcacatctgacctaatctagccatgacaaagcataccatagacttgcagggaaaaaagagagagatgccagcctcctttccacctcgtgggaagtgttctgcttctccgggtaactctggacattaaaactggtgtttgtttgacctaaaatcatagatacagatgtgcagccaggtagagagatgcccacagttgactccatctcagtgcgattcgactgaaacgttatatgccgccttaatgaaggtatacatgcattttaattagaaatccagcccagatgtaaatgaacaggtcaaattacacagcctcgcccgactagaaactgctggtgtactctgtctctgtcttcccgtcctttttatgctaatgtttttcttcgatgtgctccctgcatgaggcaagaactaattctcttttaaaaatgatacattaaatagatgaaatggcaagctaatgaaattataaatctatattataaataaaataatagcaggcccaatcctgttgaggtgaaatgagccgattgtgctcatcagaggcagttggacatttttgtcctcgcatctggctggtcatcatgaattactctggagggagagatgttgacctgtctaaccaaaaaagcatttatgtctctgagccagcactcccttctctgcggccagcagactcctctaacgaggggggtgtcttcagcaactgggaggtagctcatcttggcaaacgttgttgacacaggcatctctccgagtttccaattttggggtgctgtggctctgggggaagaaaagcaagcgcttgcctatactgtgctaaaccgcattaaaaaaattccaacagaaattgtgacgagggaatctcaataactcttaaagcagtttgttttgactaactcgagcattacagtgggatttttctaactgaccatgcaaatatgtgtttcctgatggctgtctgtttcaggcaggctagtgagctagttcttcaacggtatttcattttcttacttgcagggctaacttaaaagagttttttcaatgctgcagtgactgaagaagcagtccactcccatgtaaccatgaaagagagccagagagctttttgcaccatgcatttttactattattttccaatacttagcaccatttcactaaggaaccttgaatacaaccaggatcctcctttgcatgcgactgtagctgcatttcatgaatagtttgaacccttgtcaatgcattttttgaaaaagaaagaaaaaaaaaacttcgtgtatgtgactcaaagcatgtaaccttaagatgttgcattctaaactgacaataaagacctttcccaaatatgctggtgttctgaggactgtttaatatgctcttctaactcatttggaccagaacaaataagcctgtaaataaagcgggaatatacacactttccctcacctagggagaagccaggccaaggcagggtgtgagagttcttgcatgcatcgcactgaaccagcttattttaaccttgcaggcgataaaagcgaaaatggatgaacttaggcctttgatacctgtgttggaagagtacaaggccgatgccaaattggtattgcagtttaaagaggaggtccagaatctgacgtcagtgcttaacgagctgcaagaggaaattggcgcctatgactacgatgaacttcagagcagagtgtccaatcttgaagaaaggctccgtgcatgcatgcaaaaactaggtaggcccagtaccctgcgggacgtggcgctgcactgcccacctccggcacacgcacaggcttagggagtggtgctgaagtggacagcgcccgcctggcttcgcgaggtgatggctggattagggctcctgggcaggtctaccttgagagacagcaaaggaggagcgtaggccacacccatcctagggcattgttcagagccgggtcttgtgcagaggccacagaccccgctgagtcgcacatctggaaaaaaatagcataccatctggcagattgtgtgtgtgtgtgtgaatcgtatgtgtgtgtgcattgaagacaccagtttaatagggctggcaataacatctcagattcctccggattgagaacgggggctggtggagctcctgaaatattgaatcatgcatagtttgaataaaaaagggaacaaaattcaatcacatctcagtagagctgccattcacagcacgggagggagcccctgctcacagcctggaaggggaggagcctctgagcaaatgaaccccttcctcgggtgtgtttcctaagaaagacccccagtgtggggtgacccatttggattcttatttctgattgattacccattcaccttatcaactttccagttaattactaggagaaatattaacacattagtgtctgagtctgcttttaaatagcacatttcaaatcccaattccacttttaatttttcttaagaaatattagccatccgtcctcaccaagctgtttttgttttttgttttttgtttaataacacaaaggttgttgttttcatactacctactttttagggtaacttagggtaattttagggtgattttgccttatgaagtttatctcaggctttctctgatgttctaactggattacctttttatttctactctcccttccacacacacacactccaagtgccttactataaacctaaaacatccaaaaagaacactttaaaaaaaaacctctaatgttagtcaggtgaagagagagaatattcaaggggaaagaaaagagtcagccaaacccttggtttcccaggttcccaatttaggtgatttttaaacgctcttctaggtgtcctcagtgaacgcacaaagctttaaattccccagtcccgaaacagagacaataagaaatgcttggagtcagagaactaatccattttgatgtgtgcatgctggctgtgtctcacgggggctgctaactgcattctttcagctctgtgctccatggtgcccagccctggtatgacagtggctgggtgatctcagacagtcccaggaggggggtctgggccagatggcctctaccatggcttcccaatttgactttctaggagttggtgtgagtgtttgactttctaggagttggtgtgaatgttaatgatgatagggctgtattaacgatcatggggctatataactattgcccttgggtactgtcttcggcttgctacccatcgtgaccttgagtgacctcaccattgcccctctctccaggacctccaaacactcccacttatggggtctccctcggcctccgaagaatccagtggtggggtctttttgttgttgttatgtatttccccatgagcagtgtcttccctccctggaatagacagggcacatcatggagaaacccagagaaaacctccacattttccaatcaaggaatcaagggagcaaagtgaggatttggagtaaaaagttgcaaatagtgagaagccaagggcctttctgagggggcaccacccctcccggatgagctggccttcccatctgtgcttggcctatttttagtagctgggtgagtttaccttgcagcttaaaatcttgggtcttgtgaaagagatcacatggccgtccttgggctgctaacaccattgatttgggagtaaagagagagggttaacaggcctccagaggccctctttctcccacctttggagtgcagctgcctggtttcacctctgcgtttaattctttctttggaagggctgcgacacgtaggcggagctgctgaatcagagcgtctgcaggtggggccctggcatctgcccgtagccagcacccagctaatcttgacacacagcaaactgagctcccccgttagaggaggcagttttgtaaggtagtaaatgccactcgatagctgtgcgactcccccaaggcatttcccctctcgagtttcagctgcctcctcctctgtaaaatggtgcagggagtccctgagctctggggtaattcatggaatggtaattcatggaagtccttcagatcctgagctcagcactgtgcccagcagaggaggtgctggtaggcaggtaggtcgatggatagagaggtgatgacagctgactgataactggataggtagttagatagataaatgattgattgattgattgattgattgatagatgattgacacatagatagagttagcagctgctgaccttcctcctccctcttcccaggctttcattcaagctcagtaacttaagcaccaaattaatatcctgcagctgcttaatttagcttcattttagccttgcaaataggtacccatctcaggaccaccctggcagcctgttcccttgctgggaggttgctgaggagcccggccttctgcaaggtggagccggcacccgctgccggccgtcctgggtggtgagggcttctgtgagacgtggcctccagtagtgggcagtttcctcccctgtcccggaccccgcagtttcttggtgtgcctgctgttcctgctggtgtgaagtgtgactgcaggttccatggtctagtgaacagggtccgggttcccttcctgcagtaagcctggaggagaggctctcccaaagtcttcctgatagttctctccttgctgctaccttcttcaagtcactgttgctgacaagcccagaacatactcagaaacagagaaaacaaggaccagggctccatcagttttcagtttccaattctgaagatccctcttttgatggcattatatgattttcctaatggcccagccagaaacttgaatctgaacctctcctctcgggcatcctctggaaaggaggcagtttgccggttcccctggagcaagtcttgttgccaggcaaggggagagtcagtgctgccccgtgcccctggcctcgtcccctgagctgggcgtggggccctctctccaggggagcctgcagagtcagatgccccagcagcaaagctgagcgaagccagaagcgtgagggtcagtgcacgatgctgctctacccatggagctcccggggcgtggcatgtgcctgtcagcttcaggcctccgtggtcctctctataaatgaggggtgagggggccagctgatttgaagggctcctcccagatataccatcctggcactctggtgctaaaggcttccttagtttcttttttttcccaaagcctgggaagcccccagacacccataagcacagacagagcctgggcttcagggtcagtcgggtctttttgtttcactggaacgcgtcttaacaaagcccgctggctccagcttcagagtcccctgtcagccgctgggagctgggcctgcctgggtatctaggttgatgcaagtccagttgcaggcccccgtcccagctcgggatggcagggcatagtgcttggctctggcaccatgccctacactctgtcctgggggagacaaacctgaagcctccctcttgttccctgaccctgagatgtgagaaagggtcagccaggcagagagagggtctgtcttctcctgcccccggccctgagtggagctagacagtgagccactgtcaccagcacacatggtgcgttcaaggatagagagttaagcccttgccagtggattctgaaggaaaacccactagagtgacaggggaggaaataaaccaaaatctaaaaagccgctcagaagcactgactgagtggggcccgggcggccagggcttctcccaggaagttctccagatgacaggcggtgagagtctcctggaggcccgcccttcctgccatgcgaggaagagggtctgccgtgaccctttcctcagtggccgccccagcccaggccctgggtctgatctcgaggcctggcggggatgcagcatggttctcagcttctttcatctgcattttggacatcggtttgtccaagttcccagctttgcagacttcttccagcttaagtcttcccggattgcaggaggtaaaagctgtgaccaagggagataggctcagaggacaggcagcaggacaggaaccccatctgcctgcttcagggcactcagccctcctggtctgtctcctttgttgcccagagacccctctgtctctggcctgaggtcctcagtgggcatctccccgggcctgttctatgagatcgtccatgctgctgcgtagaagtggcctcctcttgtgaatagcaagaggcagattgtctcatccgcaggggcatcagcttgtccaggctcccggccttgcacagcgctctgccctctgagcccgtctggggccgtcggggggcctctgcccctccagctgagcatgctggccctccaagcccaggccttcctaggagagagagcccgtttgggccatttcctgagcctccaacagtgcaggaagcccggccagccctctcctgcctctcccctcctcttccctccttgtgtgtgcatcctgagtgctcgtgactggagagggacgctttcctgaactgcatgtgccaagattccactgaggctctgccatgggcttttttggatcctgtcagttcctgaggtcttggcagaagcagtctggatggagaaccaaaaataactccctgactcaaggagggcaggtggcctccagccccaagggccctgggagctgtgcctacagccagcagttggaagatcagggtgcagagccagcctcacccttccctgcccttgctaagccaggattttaaggctcatttcaaggggtcacttttgcatttaaaagagggagctggagagggtgatgctcagctctgagccagtggggcccatgcaggagggaagagggagcccttggcccacggcagggtgggcctggggcagagccgccctctggagagcagaactgcaaggtccagggtgggcgggatgaagtgggaggggtgaagaccacgtccacttgggctcgccttttctgcacatcctcaggctgaatccccagtgatgcctcctgacctctgtggagctctgactctgtggcaggtgctgtcccaaaagctctctggtggtccctgccctaattctcacagcagccttccgagggggcactgttgttattagcccatctatagagaaggagagggagtacacagcagcccccagggaagagggtcaccctccctccttcactgagacagagagtgaagccttcgacttgggaggcttttctgagcaatgagtcattcgttcattcattaattcattccatacaaatccccaggctgagttttggaggaaacagggtgagtgcaggaagcttcttccctggggcagtaagacccagacattccttggcagccccatgagatgtacggtgggaagctggctccggctgcagtgaggacaggcagaccaggcaaagtgaaggaagggcacgtctttagggcagaggtgtcagtttggggcaagccagtgaaggtgggtgggaaggtggggctggggccggcacaccaggtggagggacagctggagcaaagctgcgtgaaccagcagggcggggctctggccaggcggcggagaggagcccagcactgacttgctgcctctggcctctgccggctgcctgcaaggtggagggcagacacctggtctccttccggtaggtcatgcgaccgggatgagtctgctgcgggtgtggttccgggcgttagtgtgggccagcgtcacggaaggcccggcgtgttgactgaggaggctgaagtggccagagcccgtgtgcgctgctggatggaaacccagcatggggccgccactcctctaggcttctccttccggacaactaacagaaccacggcgtggaaagtcctcacgggatgttcacagggccaaggcactgtcttaggggacaccagcctctggatggcagggagggctggagaggggctgtgaagggcttctcccagcgcccacccagtgcagagggagctgctgtctcccccgaagcccagggccccccagcagccggagggtgagcccagccatggctcctccctcccacctcctgcctcctgctcctccagggccttagtgaagccgccctgagctccacctctccgccagcgagttgcactggggtgaaaatctgggccgggctctcctggaagaggagtctcttgtgagacttctcagaccccccacatcttcttacttctcgtccccatagacgccggtcagctgtggccatctcctctccatccctttcttctgctgcttctcctcccacagatggggagcatggcctggcccagagcccgtgtggaccacggccgagaagaccctggcagcctctcacccggccccactgccaggaagcctccagccatgaggggacaatatttacttgggaaagcacataattcctcctgaaagtaggaatggggaactacgcacggaggaggaagggagggaagggctgtgacatttcttcttccaatcggggcagaggcggcagcccgggagccaggttgccggggccttggagcaatgcagcccgactcgatgggaatttgggggcaaacccagtctttttctgtgggcagtgggctcctcgtccctctgaaagccctggtgcccggatgcacgctctcccaccaacccacagaatcggagacgcctccatgccggcagggccgggagcgttcctctttctggtcttgtgtgatgccagtaacaggccacttccagttgggagagagtgggaggcaccctgagacccgcgctgagcatgggagtggccaggccgcgtcctcccgggggccagcctggagcctgccccaccctgcttgccgacaggattatcctggctgagccgaggtccgggcgcactcacccgcagtcttcccccacacgtggatggcctctgaactcgtgtgctcgtgcaagcccaccgagggtgccacgcagacctaggaggtcacaggcgggcttggccgggagggacagggcagggtgcggggacttgtggtggaggggcccttcctctaccctccccaggaaagccacccacttcccatccagggtccctgttagaatcaggagcgtttggcccttcagagggcggccgaggctcgtgcattctggaagagctgcgtggctgcgcagacacaccggcctccgggcaggaggagttctgttcctgtgcagtgggtgtggaagccgccctgagccctcgggggtgtggggggctccagcctcagtccaaatcaccgggtggcccgggtgtgtccctcctcacctccatggtcctctgtgagacgggagggctgctgagatcacatccaggatcccatccgccctggtgcgctccagcttgggctcctcccactggcagaagcaaccggctcacgcctgtgggagaagcgcgcaggctcttcctgcgggaaggcagctgtggcctttgtcccgggtcagttccggggcggctggtggtgctgctgctccatcgtgggacagggcctgcctggatgcagtgtctcacactgggcctgactgtgccccacgcgggctgggctgtcagggcatgacctctaatgcccttgggggtggagccgctgcgttcccatctccaagagaagaaaattgagtcttggcaaagctgaccccctctcctagaggtgctgctccagcacccccatccccaggccatgggtccagcaggctgaggaggcgtgaagcctgggagggcggccgtctgcccacggctcttccctgctatcctggccacagctgctctgttttggggaggaggtggcttttccgagagtgggggagttgtctgtgaaaacaagggcgtgagcagctttccacagtaccccagaccccgagggcaagaggagaagccgccacatggcacgtgtgctctgggcagtcgaggtcagggtcatcaccgcgggccggggcccccgccctccctctcctgacctgcgtgctcttttccagcttgcgggaagttgacgggcatcagtgaccccgtgactgtcaagacctccggctcgaggttcggatcctggatgacagaccctctcgcccctgaaggcgataaccgggtgagtgtccccttatgtcatagggggtcatttgggcaagggcgctctcggacacctggtgggccccagacatgggtacaagccacgcccaccctccagggcctatggactgggcagcttggtgcctgggggcgtttgttcctggaagactttcgggagggacccaggcctctatgctaatccagagctgtagatcatgggccagggagtgacatgaggttgatggtatcccatgacatggctgtagaccccttcaaggcctcctccccggcccggtgggctgggctgggctgggctgggctgggctgggctgggctggtgccccaagtccatttctctggagccgaaacccagccctgacttttccggtccttgcatctgcttcaggaagagaattcattaggcctttcttgttttaatgacatctcatttgtatttcatttgccattcctttcatggctgatggagactcatgttccttttgatttaggaaagaggccatgttcttttttcccaggcttttccccgctgggtgaaacctcagaagaggagagagaaacaggcatcatatttgcctttatctggggggctgggttttatgttcgagcccctttgaaaaatggggaggtgagggtggctgcccgactgatggtgaggcccggcctcgctcggcccctgggccccagacccctgtatacaggcagcatgggcttgtagactccctccaaagtgagcaccctctgaaggctctcgatgggggagggaagctgtcagggcttcgctgtcccttgatttgcaaactgacctctggctgccagagtgggcatttctcacccagcaaccccttccttcaggggtttgcaggacacttagaaaataaacacttaaaaacaaacaacccagctctgccctgggccgactgagaaaggccctttgaaatgtgagatcctctaagctttatctggagcgggtttgaaggaagggatggacccagctctctcccctctgatttctgatctctttgcctccctccttcaccattgccaccatttccacgaaatctcttatatttaaaacatgggcggtacggcctctggcacactccttccttccaggaagatggctggggaggggagggggactggcagacttgctagagcctgttgcatgtgtcttgatcccccagcctcgcatgggagtggcccccgcccccatctggaagggctggccagactgcagagccgggatacaattggtgttgggtgtttgtcagggaggtttttctgtcctgttttctaaattgtgacagctgaggcttggaagttttctaacaatttaattagctgtgaagactctgaccactcttttattccacatcccacctggaaaaccccacttatgttcagagttaggaacttgctcctgccacagatgtttttgtaatgatcataatacagaagaaaacaaggtgatgctggcaggcacttagaaagcaggcccgggcacagtggctcacacctgtaatcccagcactttgggaggctgaggcgggcggatcacctgaagtcaggagtttgagaccatcctggccaacaaggtgaaaccccatctctctaaaaatgcaaaaattagccaggcgtggtggcgtgcacctgtaatcccagcgactccggaggctgaggcataagaatcgcttgaacctgggaggcagaggttgcagtgagccgagatcatgctactgcactccagcctgggtgatagagcgaggctctgtctcaaaaaaaaaaaaacaaaaaaaacagcgagtgtgtcctgtgtgcaggtgctgtccaaagcactttcttttcatgcattaactcccttaatagcgggggaaggacggggccttcatgcagccatctgcgtgcccttcctgctttgtcctccctgggacctgcctgcgtcggggtcattggcatctcagtgtggatgacaagactgttctcacgcccagaggcagaagggtctcaggatcatggagtgcctgtctgcagcatgcactgacactccaagccgagtcccttataccaccctccccctacaagtgcccttcccacacctccccgcaatctggcccaccccactatgcagagcaggaaaaccaccccacaaacccacgctgaccacattgagatctgtgagcaggaagcagtcacctccctgctgcagagggcaaccccagggctggactgctctgtctgtatctaacaccccccaggactggactgctttgtctgtaccttacaccccccagggctggactgctctgtctgtatctaacgccacacgtgtgtgcctcttgacttctgtttttgtatcaccttcctttgtcgtagggagcactagtcaagttatcgtttttctaggtaccaaggatctggggctcctggaagccccggctgtttgctttgcacagcgcagcgagggtgtgggtttgcaaagctgtcgcggtgctgatggatgcttttgatcattaggcatctattttctgcttgatgactggatcgccctccaggaaaagaggctctgatggtgggtagagtgtggggagaagaagccgcaggagaagggatccctggcaagggggtgggggagatgggtgctggggctggcagaggagccacccggagcctgctcagagcaccctcggctacggccattccaagccatacgctcagcacagcctccaactcccagtgtgctaagtgacatgtccccaggtcctgtcattgatctaggccagccgtggtttgtttgttttgatcttttccaatctcccattcagttttgatcacacacacaggagcccaataactcgctccaaaaaaaaaagaaaaagaaatcaatgtcggcttcagtgcaggctgccacggagggcattgtaaggcaggtgtggccaagggcagaggggagaggttcaatggggctgcctagtagagagcccggctcagggggacctgcagaatctctgtgttcgtgtgaccacaggagggacggtcctgagccagcttcagggccttgctaagctgtcagaacaaggttagcttgggacgcctttgcagcaggtctttctggattcatttattaagcactgactgtatgtccggcctgtgctgagcactgagagcccccaagttcttctcagcaggaagccacccctgcgcttcccagggcaccgggccccacagcccctcctttccttcccgcccgtcccattgcctcagtgttggaggagggaagggctgtgtggaggttggccagctctcaacctgatgcgtgaatgccctgccaggctgtgttcccacccactgggagaccacatgagttctgggaccccttccccagctcggaggcttctggcctgctctgaccatggcctgccttccagagcccaacatctgcctcctcctttctgacccgcttcctataaaagtctcaggtcagctcacatcagacaccagacaccaaaaagggggccagcgctactcccaccaccccaggaagttcatgtttgaaggggtgatataattaggaaaacactgtcatttacaaacagcagcaagcttagcttagcgtttcaaagtcctttcccatcatctaaacagcgtcaaggccatctagatattttataaggatcccacggaatctttttttccagaaggtggaaatatccaaaatgcataaaaccgtgtgggtgcaggcagtgatccgcccgccgccagtggtgcaataaacatcaacgccccttccagtcacttctcagtgtgaggagggaccttcccaggaccgagaaccgttgtctgttccccttcagccttcactccccaagtggagcctccggcagaaactgccagcatcccccccacctctttcccaccacccattcagctacgggccgctttagagtgtcttttccagcggcccaggctccttgatgttaggaattctgtcaggacgacatgtgacagatgcttgccaaaccaaagggaagtgccagcgcgggcccaagtccctacgccaagaccccttaaatagcatccgcttcccacttcccgggggctgcatcagaggcatttgggacggcagtgccagatctgtgcccatcgtccagctctaccctgaaccgagaggccctcttcactgggaagctgatgaaagtaatcgggacagttaggaaaatcccacgttctcagtaaactgcaccacatgtggctgaaatgcggtgcagaggttcagacagccagtttttcttatatgggaagctgtgttgggcctcggtttcggagaagcctggtcccctgaaagcatggtatttacaaatgcattttgtgtttgcgtgagaaggaagaacccacccgttgctttatcttttccggcaaagaaaccctcatgggttggggaggggatttgggcaggaacttggggccttccagcctgtgtcgtcgaattagagtgaggctgtgctcggggcagggtggcctcgctcgccgtctggccgcaggctctcagggactgtagcgcatggcccattgaactggagaaggatgatcagtgtgcgagacctccacgacccccaggaggcctgagtccagccctgaccccctgcttttcctagatctcggggctgggaatctgaactttcctgcctgtgctgagggtgggtgtgttgcggagcttggggacatcagatgtcaccagtgagctcctgtgtggggtccctgtgggtggcgtggttcagagctgggcacagaggttcggagctgctccacactggagtctgcgtctctctgctgcccctcccagctctgtgacctttaggatattctttttttttgagacggagtcttgctctgtcgtcagactggagtgcagtggtgcaatctcagctcactgcaacctccgcctcccgaattgaagcaattcccaggcctcagcctcccgagtagctgggattacaggcgcatgccatcacttccggctattttttttctgtgtgttttttagtagagacagggtttcaccacattggccaggatgatctccatctcctgacctcatgatccacctacatcggcctcccaaagtgctgggattacaggcgtgagccaccacgcccagccaggacattcttaaccttctgttccttggggtcatgatcagaaagggcacacctgcacagggggctctcagcatacgttgtgggggcagcaccagtggctctaatacaccaggtttcagctcatggcacacagcagcccgtccagctgtgaatggagagcagttaggtcagggagggacatgcactggccgctgatcgccaaaagggctccatggaaagacggcaccaggggccacccgccctgtggggctctcagttgggagaggcacctgcagaagagtctctggtctctgctgtctaaggtccctgttccctccaggctgcactggccttctgttcccagaatgttctctctggtctccccttcctatgcctggggaaccccaagattcttccagcacgaggatttttgcgtggagccatggcccctcatgaggtcagaagctttgggaggacagggcctgtgtccccagcacctggaaaataggagatgaatggtggcccttgatggagcttggggcccagccttggggctctgtgtagccaagacctggggcaggatggccagccagccatgcacacctgctttcaggcaccattctcccagtcggtgggtgacccgtccacccagtccgcacagggccactcttgggcgggcgtctcctgggccgctgtggatgccttgaccctgggagagctcagcagcaagggctcgtgtggacaccgtggtgtcctgggaggatggcggcgtgagagcttgccttctgcaggggcacagacggaccaccacatggggccgccaagtgagattgggacaaggtccctgagggggagaaggaagggggatgagaagaggccatggcaggagggaagagggcatcactgggggcatccagccagggatgagcaggtgcggggagctgctgtctccttcatagatggaagccactttgtggcgtgggggagggcagccttgggaacctgggtggaagcaagctgtgtcggggtcagggcaggctttggatgtgctgtgtgcaagatccagaccagcgtgcacgcttcctaatgcacagagcatgtggcagctggcacagctggccatgtgaccctgagagaggccttccttctctctgggcctctctgaagtggggatttggggacggggtccctaagcctctgtgactctgtggagggatgggtgtcacggcctgtcttgctgcatcctgggctagtccggaggccaagccccctgtggtctcggttttcagatgggctgaccctaccgccacgtccccacaggtggtcttatggccttcttcctttgggtggtgcctgggcctgatgagctccagcaggctctgagggccgtgtctagcctacccagaaccaccaagcccactcgagtcccgggctaaggacataaatgatgatagctggcatttattgaaccactacaggtggcaggtcctcacaggacctattttacagcagagggaactgaggcacagagaggggtggaaactccccaggtctcgctggagactggcggtggctggcggtggaggcgcttcttggtgccgcattaacaggagtccagccacgtggacacccctcactctgtgggatccacagagcagtgcctgggaggccagaaagctttggcagacccacggtgtgccctgagctgagtgggggtgctgggtcacagtgaggcggcaaggcctcccctccgggctcacggtcaggtgggagacacatgcccgatgccccagcacacgggtgtggaagcccaagaggtgccctgtggctgctgcaggagctcccacagcctggggggcaccacaggggctcactgtcacccagtgctggaggctggaagccccagaccagggcgcctgcagggctgcgctctgaagatgccaggggaggaccctcctgcctcgtctcgtggctccatgtgttcctggctgtggctgcatcactccagtctctgcctccgtcttcacatggctcctccctgtgtctgtgtcccctcttctgtctcttataaggacacttgtcactggatttagggcccaccttcatccaggaccagctcatcttgagattctttttttttttttttttttttttttttgagacagggtctcactctgttgcccatgctggagtgctggagtgcagtggtgcgatctcggctcactgcaagtgattctcctgccttagcctcccgagtagctgggattacaggcacgtgccaccatgtccagctaatttttatatttttagtagagacgaggtttcactgtgttggccaggtctcgaactcctgacctcgtgatccgcccaccttagcctcccaaaatgctgggattacaagcgtaagcccccatgcccagccgagattcttaatcctatctgcaaagacctttttcccaaataaggtcccatttgcaggtcccaggatgtggacctatcttctgggggccactattcagtctactataagagtggacacctgcctgcctgggggtacaggggtttaggaaatacctcccaagggggtgagtgtggcctggggcagggatggggacggggtggaggggcagctcccgcaggcacagaggcaagagttggtaggtctgggactgaaggatgtgactgggcagcctggggagggagggaggggctggctgggcagcagagtttggagtttgggatcagaaagggtgtgtacaggggactgggtaagggacaggctggaccggcattctagaaggttcacacaggccacaccagagctgcagaggccaggagggagctgccagcaccactgaggaatgaggcagcgggaaccaggccccagacacggattattcccatgtgcatggggaatagccctcttcgcacgcggcacagtgtggaccccggagctgatgtcatccaagccccccacccacccctgctctatggcaggaaggatgaggccaggcaggaacagcaagcttctccctgcaaatgcgtctgccctatttgggacaatttccccgctggagcttgacacacacagatcctttcctcaagaccgctagggccacaaaaggtcaagggtgaatgtacttctttgtaattcagccctgctaggaaagaaatatcctctattgtgggagccaccgaaatcttcagccaggggtgccatcttcaccttcctacacattcaccactcaggtctgcagcttcaggccgagccttcaaacccacgtccacatccaggttgcatccttcggaagagggaggaggccggcgaagccttacctggccaggccccacttccccagcacagggacgagattgcttgcccaaggtcagccctgatgggtgacgcagggacagagggtttgccctccaggttcccctgggggcagaaaggataagtggacggagggaaatgggccgtttacctggcaggttcatggcatggacagcagcgcatcagggctggggcttttcagcctcacccactgacgtgtgggaccggatcatgcctcgttgcagggggctgttggtgtgtggggatgggtagcagcacccctggcctctgcccctagatgccagcagcactttccccacttcccagtcaagacaatcgaactgtttctgggcatgtgcagtgcccccagggcagatcaccactggccagattggtggtttcaagctcagggtttggagtcccctcttgactctgaccactggaaagtcaccaaacctccctggcctccattttctagtctaaaaatggggcgatcacggctccctgggtgcacgggctcatgtgataattgaaggcaagagatgatgccagcgtccggcacagtgaatgccctgagtaacggcacacaagtgacctggcatccaggcagcctggttccgctcttggccttttgctgggtgcccttgggtctttttggaaagaacgataggtcctgcccggaggcgcaagtgctcaatctccctaaaagccggtactgtatttggggccgcctccccagagaggaagctagcaggcattgatggaatttgatctgagccttgggacttggagaaaggggaagaaaaaggcctttcagatgagaacattgagggaatgaactcagaggaggggacctactgtgaagcacggggctccttatcccaaggcctgtggggttctgagtgcctctctttgcatgggtgccttcccattggcaactggatctcagcctcgaaggagtttttaccccacagaaactggcaaacgctcagagcggcctcccctgaaccctgcctgagcctgtgcaccgtgtgccgattctccctcccacccccaattacatcttcagagtgcggtatcctgtgtcattttgctggtgtactggaagagaaggtgatttaaaaatccttagcaagttgatgctggtgtatcccccttctgcccatgggaaggaggctctggacccagggtacaggggtgagtcaccctgggtccccgcgatcaagggcttgtttgtgggggtgatgtgtgaacacagtcaaaggtgttctgtgtgctgcgatggcaggggtggttcccctccaggcaggtcaggagagggcttggggaggaatggtattagatgtgagtttcagaagcagtgagctccctgtctgatgagatagccaagcaaagagggggcaagagggcagctttgtggcagtgtcattaagggggtttcagcacatggccccgttccctccaatacccctcccggttctaatctgtgacccacatcattgaggtttggtctgtgggaggctaaggataatagcctcaaatgaggcccagctgaaaaaaaaaaaaagatctcattaaaaacaaaaagcacatctgcttgtaagtatcgaatggatgtcttgagaagaaggttataatttttttttaatttttagcgcgtgtgtaatgccaagtctgaaagctccctcatccttagtctcctgcagctccagagccctcgacggataaagcagctgtctcattgccagacagatgcatgcagagcggcaccagcctgccagactccctctgcctaactgcgttgctttctaatttgctcccacattggttgaaaatgactaaagcattttgcgcaaagtccagacagttctcaagtcaactggcatttcatcggaaatctcttcttctgtaaccccaaacttgggccattactgggtttgctatcttggttgctttcgacaaccagaggcttcttcaaagcccatattcttctcggagaggcacttttgctggattaggggtgacaatgagtgattacactcacagcatcccaaatgccaaattaatgacattccgccctgcagacaggatgactcagtccgtgccgcagcgatggcttggtgggaggagaggccttgagcgtggtgtcttgttgggatatgggagctggcccgccccggtgactttgagagtcgatcctcactgcagacaacagctgcctggggctgtgagcatttgccctcggctaggccaggtggctgtgccccttcgaaggcccccttcagccctgggttctgtactgaaggagctcctttcttgcacacgtgtgtatgacccccactatgggccaggcccctggctaggggtatggcagtgaacagataggtgcagctgctgccccacaacctggtgtcatcccaagcaagcagagggcaggattaggggcgaatggctgcacgtatacgcctgtgtgctcccatcccaccttcctgctagattctttcttcaaactgggcctgtgtgtcccatcccaaattcccactagatcctttcttcaaactgtgtgttcccatctcaccttcccactagaccctttcttcaaactgggccatcctttctctgatagcacaatagctgtgtctccttttgaggagtgtgctgggtttggaggtaggacgtggcaccctttggttgtgactactctttcctggctctctgggtttggagggaggacatggcaccctttggtcgtgactactgtttcctggctctttcagctccttctcctgaggtccaggaaattccaagtcggggaacagcttcccgcccaaggaacagctttccacacaagcctgttccaccccactttggaagtgctcgccgggtctttggaagtgctcactgggtctttggaagtcctcactgggtctttggagtactcactgggtctttggagtgctcaccgggtctttggagtgctcaccaggtctttggaagtgctcactgggtctttggagtgctcgctgggtctttggaaatgctcactgggtctttggagtgctcgctgggtctttggagtgctcgctgtgtctttggagtgctcgctgtgtctttggagtgctcaccaggtctttagagtgcttactgggtctttggagtgctcactgggtctttggagtgctcgctgggtctttggaatgctcgctgggtctttggaatgctcgctggatctttggaagtgctcactgggtctttggagtgcttgctgggtctttggaaatgctcactgggtctttggagtactcactgggtctttggaatgctcactgggtctttggagtactcactggctctttggagtgctcactgggtctttggaaatgctcactgggtctctggagtactcactgggtctctggagtgctcactgggtctttggagtgctcgccgtgtctttggagtgctcgccgtgtctttggagtgctcgccgggtctttggagtgctcgccgggtctttggagtgctcaccgggtctttggagtgctcactgtgtctttggagtgcttactgggtctttggagtgctcgccgggtctttggagtgctctctgtgtctttggaagtgctcactgggtctttggaaatgctcgccgggtctttggcatgctcgccgggtctttggagtactcactggatctttggattactcactggatctttggaagtgctcactggatctttggaagtgctcactgggtctttggagtgctcaccgggcctttggaagtgctcaccgggtctttggagtactcgttgtgtctttggagtgctcaccgagtctttggagtacttaccgggtctttggagtactcactgggtctttggagtgctcgctgtgtctttggagtactcactgggtctttggagtgctcactgggtctttggaagtgctcacttggtctttggagtactcactgggtctttggagtactcactggatctttggaagtgctcacggggtctttggagtgctttctgggtctttggagtgctcactgggtctttggagtgctcagtgggtctttggagtactcactggatctttggaagtgctcactgggtctttggagtactcactgggtctttggaagtgctcaccaccgggtctgctctctccacaggtgtggtacatggacggctatcacaacaaccgcttcgtacgtgagtacaagtccatggttgacttcatgaacacggacaatttcacctcccaccgtctcccccacccctggtcgggcacggggcaggtggtctacaacggttctatctacttcaacaagttccagagccacatcatcatcaggtttgacctgaagacagagaccatcctcaagacccgcagcctggactatgccggttacaacaacatgtaccactacgcctggggtggccactcggacatcgacctcatggtggacgagagcgggctgtgggccgtgtacgccaccaaccagaacgctggcaacatcgtggtcagtaggctggaccccgtgtccctgcagaccctgcagacctggaacacgagctaccccaagcgcagcgccggggaggccttcatcatctgcggcacgctgtacgtcaccaacggctactcagggggtaccaaggtccactatgcataccagaccaatgcctccacctatgaatacatcgacatcccattccagaacaaatactcccacatctccatgctggactacaaccccaaggaccgggccctgtatgcctggaacaacggccaccagatcctctacaacgtgaccctcttccacgtcatccgctccgacgagttgtag

(19) United States
(12) Patent Application Publication
(10) Pub. No.: US 2003/0215852 A1
(43) Pub. Date: Nov. 20, 2003

(54) Novel pancortin-Pablo protein interactions and methods of use thereof

(75) Inventors: Robert John Mark, Lawrenceville, NJ (US); Andrew Timothy Wood, Newtown, PA (US); Kamalakar Gulukota, Hyderabad (IN)

Correspondence Address: Bill T. Brazil, Wyeth, Five Giralda Farms, Madison, NJ, 07940-0874, US

(73) Assignee: Wyeth, Madison, NJ

(21) Appl. No.: 10/395,740

(22) Filed: Mar. 24, 2003

Related U.S. Application Data
(60) Provisional application No. 60/369,244, filed on Apr. 1, 2002.
(60) Provisional application No. 60/386,645, filed on Jun. 6, 2002.

(51) Int. Cl. A61K 48/00; C07K 14/705; A61K 38/17; C12Q 1/68; C07K 16/28

(52) U.S. Cl. 435/6; 435/69.1; 435/320.1; 435/325; 530/350; 530/388.1; 514/12; 514/44; 536/23.5

(57) ABSTRACT

This invention relates to newly identified human pancortin polypeptides, the interaction of the pancortin polypeptides with a Pablo polypeptide, the use of such polypeptides, as well as the production of such polypeptides. The invention relates also to identifying compounds which modulate the activity of a pancortin polypeptides and/or the interaction of a pancortin-Pablo polypeptide interaction, wherein modulators can be agonists, antagonists and/or inhibitors of pancortin and/or the pancortin/Pablo interaction and therefore potentially useful in therapy.

This application claims priority from copending provisional application serial No. 60/369,244, filed on Apr. 1, 2002, the entire disclosure of which is hereby incorporated by reference and provisional application serial No. 60/386,645, filed Jun. 6, 2002, the entire disclosure of which is hereby incorporated by reference.

### FIELD OF THE INVENTION

The present invention relates generally to the fields of cell signaling, apoptosis, neuroscience and molecular biology. More particularly, the invention relates to newly identified polypeptide interactions, comprising a neuron-specific pancortin polypeptide and a neuron-specific pro-apoptotic Pablo polypeptide, the use of such polypeptides, the modulation of such polypeptides, as well as the production of such polypeptides. The invention also relates to identifying compounds which may be agonists, antagonists and/or inhibitors of the pancortin-Pablo interaction, and therefore potentially useful in therapy.

### BACKGROUND OF THE INVENTION

Apoptosis is a form of programmed cell death which occurs through the activation of cell-intrinsic suicide machinery. The biochemical machinery responsible for apoptosis is expressed in most, if not all, cells. Apoptosis is primarily a physiologic process necessary to remove individual cells that are no longer needed or that function abnormally. Apoptosis is a regulated event dependent upon active metabolism and protein synthesis by the dying cell.

Apoptosis plays a major role during development and homeostasis. Apoptosis can be triggered in a variety of cell types by the deprivation of growth factors, which appear to repress an active suicide response. Apoptosis is particularly important for the physiology of the immune system. Apoptosis is the mode of death of centroblasts with low affinity for antigen within germinal centers, cells killed by specific cytotoxic T lymphocytes or natural killer cells, as well as thymocytes bearing high-affinity T-cell receptors for self antigens that are clonally deleted during thymus development (negative selection).

The morphological and biochemical characteristics of cells dying by apoptosis differ markedly from those of cells dying by necrosis. During apoptosis, cells decrease in size and round up. The nuclear chromatin undergoes condensation and fragmentation. Cell death is preceded by DNA fragmentation. The DNA of apoptotic cells is nonrandomly degraded by endogenous calcium and magnesium-dependent endonuclease(s) inhibited by zinc ions. This enzyme(s) gives fragments of approximately 200 base pairs (bp) or multiples of 200 bp by cutting the linker DNA running between nucleosomes. Thus, DNA appears to be one of the most important targets of the process that leads to cell suicide. The apoptotic cell then breaks apart into many plasma membrane-bound vesicles called “apoptotic bodies”, which contain fragments of condensed chromatin and morphologically intact organelles, such as mitochondria. Apoptotic cells and bodies are rapidly phagocytosed, thereby protecting surrounding tissues from injury. The rapid and efficient clearance of apoptotic cells makes apoptosis extremely difficult to detect in tissue sections.

In contrast, necrosis is associated with rapid metabolic collapse that leads to cell swelling, early loss of plasma membrane integrity, and ultimate cell rupture. Cytosolic contents leach from the necrotic cell causing injury and inflammation to surrounding tissue.

Although the exact details of apoptotic pathways are not fully understood, it has been established that caspases, which are cysteine proteases (cysteine aspartate proteases), play an essential role at various stages of the apoptotic process (Grutter, 2000). In addition to the caspases, the highly regulated process of apoptosis involves an intricate cascade of events. The Bcl-2 family of proteins constitute an intracellular checkpoint of apoptosis. The founding member of this family is the apoptosis-inhibiting protein encoded by the Bcl-2 proto-oncogene, which was initially isolated from a follicular lymphoma (Bakhshi et al., 1985; Tsujimoto et al., 1985; Cleary and Sklar, 1985). The Bcl-2 protein is a 25 kDa, integral membrane protein localized to intracellular membranes including mitochondria. This factor extends survival in many different cell types by inhibiting apoptosis elicited by a variety of death-inducing stimuli (Korsmcyer, 1992).

The family of BCL-2-related proteins is comprised of both anti-apoptotic and pro-apoptotic members that function in a distal apoptotic pathway common to all multi-cellular organisms. It has been suggested that the ratio of anti-apoptotic (Bcl-2, BCl-X_{L}, Mcl-1 and A1) to pro-apoptotic (Bax, Bak, Bcl-xₛ, Bad, Bik and Bid) molecules may be involved in determining whether a cell will respond to a proximal apoptotic stimulus (Oltvai et al., 1992; Farrow, et al., 1996). Because members of this family can form both homodimers and heterodimers, the latter often between anti-apoptotic and pro-apoptotic polypeptides, the balance of these homodimers and heterodimers could play a role in regulating apoptosis (Oltvai and Korsmeyer, 1994).

Members of the BCL-2 family have been defined by sequence homology that is largely based upon conserved motifs termed BCL-Homology domains (Yin et al., 1994). BCL-Homology domains 1 and 2, designated BH1 and BH2, have been shown to be important in dimerization and in modulating apoptosis (Yin et al., 1994). A third homology region is an amphipathic α-helix designated BH3, which has been found in some family members and shown to be important in dimerization as well as promoting apoptosis (Chittenden et al., 1995). BH4, the most recently identified homology domain, is present near the amino terminal end of some pro-apoptotic family members (Farrow et al., 1996).

All known members of the BCL-2 family, other than Bad and Bid, have a C-terminal membrane-anchoring tail (TM). BCL-2 family members with a TM are intracellular integral membrane proteins most commonly localized to mitochondria, the endoplasmic reticulum and the nuclear membrane. The intracellular membrane localization of BCL-2 family members, together with the identification of structural similarity between the BCl-x_{L} monomer and the ion-pore forming toxins of colicin and diphtheria toxin B fragment (Muchmore et al., 1996), has prompted electrophysiological studies by several groups on the ability of BCL-2 family members to form ion channels in artificial lipid membranes.

Some disease conditions are believed to be related to the development of a defective down-regulation of apoptosis in the affected cells. For example, neoplasias may result, at least in part, from an apoptosis-resistant state in which cell proliferation signals inappropriately exceed cell death signals. Furthermore, some DNA viruses such as Epstein-Barr virus, African swine fever virus and adenovirus, parasitize the host cellular machinery to drive their own replication and at the same time modulate apoptosis to repress cell death and allow the target cell to reproduce the virus. Moreover, certain disease conditions such as lymphoproliferative conditions, cancer, including drug resistant cancer, arthritis, inflammation, autoimmune diseases and the like, may result from a down-regulation of cell death regulation. In such disease conditions, it would be desirable to promote apoptotic mechanisms.

Conversely, in other disease conditions, it would be desirable to inhibit apoptosis such as in the treatment of immunodeficiency diseases, including AIDS, senescence, neurodegenerative disease, ischemic and reperfusion cell death, infertility, wound-healing, and the like. In the treatment of such diseases it would be desirable to inhibit apoptotic mechanisms.

Thus, there is clearly a need for the identification and characterization of further proteins, their genes and their ligands, which can play a role in preventing, ameliorating or correcting dysfunctions or diseases related to cellular apoptosis. The identification of compounds that can modulate apoptosis would be useful in developing treatment regimens for advantageously modulating the apoptotic process in disease conditions which involve either inappropriate repression or inappropriate enhancement of cell death.

### SUMMARY OF THE INVENTION

The present invention relates to newly identified human pancortin polypeptides, the interaction of these pancortin polypeptides with a Pablo polypeptide (i.e., a pancortin-Pablo interaction), the use of such polypeptides, as well as the production of such polypeptides. The invention also relates to identifying compounds which may modulate the activity of a pancortin polypeptide and/or the interaction of a pancortin-Pablo polypeptide interaction, wherein modulators can be agonists, antagonists and/or inhibitors of pancortin and/or the pancortin-Pablo interaction, and therefore potentially useful in therapy. In particular embodiments, a pancortin polypeptide, a polynucleotide encoding a pancortin polypeptide, a modulator of pancortin polypeptide activity or a modulator of pancortin gene expression may be used to modulate apoptosis in cells, more preferably in neural cells.

Thus, in specific embodiments, the invention is directed to an isolated polynucleotide encoding a human pancortin polypeptide, wherein the polynucleotide comprises a nucleotide sequence having at least 95% identity to the nucleotide sequence of SEQ ID NO:1, a degenerate variant thereof, a complement thereof or a fragment thereof. In preferred embodiments, the polynucleotide comprises the nucleotide sequence of SEQ ID NO:1, a degenerate variant thereof, a complement thereof or a fragment thereof. In another embodiment, an isolated polynucleotide encoding a human pancortin polypeptide is provided, wherein the polynucleotide comprises a nucleotide sequence having at least 95% identity to the nucleotide sequence of SEQ ID NO:3, a degenerate variant thereof, a complement thereof or a fragment thereof. In preferred embodiments, the polynucleotide comprises the nucleotide sequence of SEQ ID NO:3, a degenerate variant thereof, a complement thereof or a fragment thereof. In yet another embodiment, an isolated polynucleotide encoding a human pancortin polypeptide is provided, wherein the polynucleotide comprises a nucleotide sequence having at least 95% identity to the nucleotide sequence of SEQ ID NO:5, a degenerate variant thereof, a complement thereof or a fragment thereof. In preferred embodiments, the polynucleotide comprises the nucleotide sequence of SEQ ID NO:5, a degenerate variant thereof, a complement thereof or a fragment thereof. In yet another preferred embodiment, an isolated polynucleotide encoding a human pancortin polypeptide is provided, wherein the polynucleotide comprises the nucleotide sequence of SEQ ID NO:7, a degenerate variant thereof, a complement thereof or a fragment thereof. In particular embodiments, the polynucleotide of SEQ ID NO:1, SEQ ID NO:3, SEQ ID NO:5 or SEQ ID NO:7 is selected from the group consisting of DNA, genomic DNA, cDNA, RNA and antisense RNA, and may further comprise heterologous nucleotides.

In another embodiment of the invention, an isolated polynucleotide encoding a human pancortin polypeptide, wherein the polynucleotide comprises a nucleotide sequence having at least 95% identity to the nucleotide sequence of SEQ ID NO:1, a degenerate variant thereof, a complement thereof or a fragment thereof, encodes a polypeptide comprising an amino acid sequence of SEQ ID NO: 2, a variant thereof or a fragment thereof. In another embodiment, an isolated polynucleotide encoding a human pancortin polypeptide, wherein the polynucleotide comprises a nucleotide sequence having at least 95% identity to the nucleotide sequence of SEQ ID NO:3, a degenerate variant thereof, a complement thereof or a fragment thereof, encodes a polypeptide comprising an amino acid sequence of SEQ ID NO: 4, a variant thereof or a fragment thereof. In still another embodiment of the invention, an isolated polynucleotide encoding a human pancortin polypeptide, wherein the polynucleotide comprises a nucleotide sequence having at least 95% identity to the nucleotide sequence of SEQ ID NO:5, a degenerate variant thereof, a complement thereof or a fragment thereof, encodes a polypeptide comprising an amino acid sequence of SEQ ID NO: 6, a variant thereof or a fragment thereof. In further embodiments of the invention, an isolated polynucleotide encoding a human pancortin polypeptide, wherein the polynucleotide comprises the nucleotide sequence of SEQ ID NO:7, a degenerate variant thereof, a complement thereof or a fragment thereof, encodes a polypeptide comprising an amino acid sequence of SEQ ID NO: 8, a variant thereof or a fragment thereof. In other embodiments, the polypeptide of SEQ ID NO:2, SEQ ID NO:4, SEQ ID NO:6 or SEQ ID NO:8 binds a Pablo polypeptide comprising the amino acid sequence of SEQ ID NO:9, a variant thereof, or a fragment thereof. In certain other embodiments, the pancortin polypeptide of SEQ ID NO:2, SEQ ID NO:4, SEQ ID NO:6 or SEQ ID NO:8 is a fusion polypeptide.

In other embodiments, the invention is directed to an isolated polynucleotide which hybridizes with a polynucleotide comprising the nucleotide sequence of SEQ ID NO:1, SEQ ID NO:3, SEQ ID NO:5, SEQ ID NO:7, a degenerate variant thereof, a complement thereof, or a fragment thereof, under high stringency hybridization conditions.

In another embodiment, the invention is directed to an isolated human pancortin polypeptide encoded by a polynucleotide comprising a nucleotide sequence having at least 95% identity to the nucleotide sequence of SEQ ID NO:1, a degenerate variant thereof, a complement thereof or a fragment thereof. In yet other embodiments, the invention is directed to an isolated human pancortin polypeptide encoded by a polynucleotide comprising a nucleotide sequence having at least 95% identity to the nucleotide sequence of SEQ ID NO:3, a degenerate variant thereof, a complement thereof or a fragment thereof. In still other embodiments, the invention is directed to an isolated human pancortin polypeptide encoded by a polynucleotide comprising a nucleotide sequence having at least 95% identity to the nucleotide sequence of SEQ ID NO:5, a degenerate variant thereof, a complement thereof or a fragment thereof. In still another embodiments, the invention is directed to an isolated human pancortin polypeptide encoded by a polynucleotide comprising a nucleotide sequence of SEQ ID NO:7, a degenerate variant thereof, a complement thereof or a fragment thereof. In preferred embodiments, the pancortin polypeptide binds a pablo polypeptide comprising the amino acid sequence of SEQ ID NO:9, a variant thereof, or a fragment thereof, wherein binding modulates apoptosis in a cell, more preferably in a neural cell. In other particular embodiments, the polypeptide is a fusion polypeptide.

The invention is directed in preferred embodiments, to an isolated human pancortin polypeptide comprising an amino acid sequence of SEQ ID NO:2, SEQ ID NO:4, SEQ ID NO:6, SEQ ID NO:8, a variant thereof or a fragment thereof. In a particularly preferred embodiment, the polypeptide binds a pablo polypeptide comprising the amino acid sequence of SEQ ID NO:9 or a variant thereof, wherein binding modulates apoptosis in a cell, even more preferably a neural cell. In certain embodiments, the polypeptide is a fusion polypeptide.

In certain other embodiments the invention is directed to an antibody specific for a pancortin polypeptide comprising the amino acid sequence of SEQ ID NO:2, SEQ ID NO:4, SEQ ID NO:6, SEQ ID NO:8, a variant thereof or a fragment thereof. In particular embodiments, the antibody is selected from the group consisting of monoclonal, polyclonal, chimeric, humanized and single chain. In a preferred embodiment, the antibody is monoclonal.

In other embodiments, the invention is directed to an antibody specific for a pablo-pancortin polypeptide dimer. In particular embodiments, the polypeptide dimer comprises a pablo polypeptide comprising the amino acid sequence of SEQ ID NO: 9, a variant thereof, or a fragment thereof and a pancortin polypeptide comprising the amino acid sequence of SEQ ID NO: 2, SEQ ID NO: 4, SEQ ID NO:6, SEQ ID NO:8, a variant thereof or a fragment thereof. In particular embodiments, the antibody is selected from the group consisting of monoclonal, polyclonal, chimeric, humanized and single chain. In a preferred embodiment, the antibody is monoclonal.

In certain embodiments, the invention is directed to an expression vector comprising a polynucleotide comprising the nucleotide sequence of SEQ ID NO:1, a degenerate variant thereof, a complement thereof or a fragment thereof. In preferred embodiments, the polynucleotide encodes a pancortin polypeptide comprising the amino acid sequence of SEQ ID NO:2, a variant thereof or a fragment thereof. In other embodiments, the invention is directed to an expression vector comprising a polynucleotide comprising the nucleotide sequence of SEQ ID NO:3, a degenerate variant thereof, a complement thereof or a fragment thereof. In preferred embodiments, the polynucleotide encodes a pancortin polypeptide comprising the amino acid sequence of SEQ ID NO:4, a variant thereof or a fragment thereof. In still other embodiments, the invention is directed to an expression vector comprising a polynucleotide comprising the nucleotide sequence of SEQ ID NO:5, a degenerate variant thereof, a complement thereof or a fragment thereof. In preferred embodiments, the polynucleotide encodes a pancortin polypeptide comprising the amino acid sequence of SEQ ID NO:6, a variant thereof or a fragment thereof. In yet another embodiment, the invention is directed to a recombinant expression vector comprising a polynucleotide comprising the nucleotide sequence of SEQ ID NO:7, a degenerate variant thereof, a complement thereof or a fragment thereof. In preferred embodiments, the polynucleotide encodes a pancortin polypeptide comprising the amino acid sequence of SEQ ID NO:8, a variant thereof or a fragment thereof. In particular embodiments, the expression vector further comprises a polynucleotide encoding a pablo polypeptide comprising the amino acid sequence of SEQ ID NO:9, variant thereof or a fragment thereof. In still other particular embodiments, the polynucleotide comprised in the vector is selected from the group consisting of DNA, genomic DNA, cDNA, RNA and antisense RNA. In preferred embodiments, the polynucleotide is operatively linked to one or more regulatory elements selected from the group consisting of a promoter, an enhancer, a splicing signal, a termination signal, a ribosomal binding signal and a polyadenylation signal. In other embodiments, the vector DNA is selected from the group consisting of plasmid, episomal, YAC and viral. In preferred embodiments, the vector is plasmid DNA.

In other embodiments, the invention is directed to a genetically engineered host cell, which has been transformed, transfected or infected with an expression vector comprising a polynucleotide comprising the nucleotide sequence of SEQ ID NO:1, SEQ ID NO:3, SEQ ID NO:5, SEQ ID NO:7, a degenerate variant thereof, a complement thereof or a fragment thereof. In particular embodiments, the host cell is selected from the group consisting of a bacterial cell, a fungal cell, an insect cell, a plant cell and an animal cell. In a preferred embodiment, the host cell is bacterial. In another preferred embodiment, the vector comprised in the host cell, expresses the polynucleotide to produce the encoded polypeptide, variant or a fragment thereof.

In another embodiment, a neural cell line stably expressing a pancortin polypeptide is provided, comprising the amino acid sequence of SEQ ID NO:2, SEQ ID NO:4, SEQ ID NO:6, SEQ ID NO:8, a variant thereof or a fragment thereof.

In particular embodiments, the invention is directed to a transgenic animal whose genome comprises an engineered functional disruption in a polynucleotide encoding an endogenous pancortin polypeptide. In preferred embodiments, the animal is homozygous for the functional disruption and in other embodiments the animal is selected from the group consisting of mouse, rat, rabbit and hamster. In still other embodiments, the invention is directed to a method for assaying the effects of test compounds on the activity of a pancortin polypeptide comprising the steps of providing a transgenic animal comprising a polynucleotide encoding a pancortin polypeptide, administering a test compound to the animal and determining the effects of the test compound on the activity of the pancortin in the presence and absence of the test compound. In particular embodiments, the polynucleotide has at least one mutation selected from the group consisting of nucleotide deletion, nucleotide substitution and nucleotide insertion. In still other embodiments, the present invention is directed to a method for assaying the effects of test compounds on a transgenic animal with a genome comprising a functional disruption of a polynucleotide encoding a pancortin polypeptide, the method comprising providing a transgenic animal whose genome comprises a disruption of the endogenous polynucleotide encoding a pancortin polypeptide, administering a test compound to the animal and determining the effects of the test compound on the activity of the pancortin polypeptide in the presence and absence of the test compound. In yet another embodiment, the invention is directed to a method for producing a transgenic animal whose genome comprises a functional disruption in a polynucleotide encoding a pancortin polypeptide, the method comprising providing a polynucleotide encoding a pancortin polypeptide having a functional disruption, introducing the disrupted polynucleotide into embryonic stem cells, selecting those embryonic stem cells that comprise the disrupted polynucleotide, introducing an embryonic stem cell comprising the disrupted polynucleotide into a blastocyst, transferring the blastocyst to a pseudopregnant animal and allowing the transferred blastocyst to develop into an animal chimeric for the disruption. In a preferred embodiment, the method further comprises breeding the chimeric animal with a wild-type animal to obtain animals heterozygous for the disruption. In still another preferred embodiment, the method further comprises breeding the heterozygous animal to generate an animal homozygous for the disruption.

In certain embodiments, the invention is directed to a method for modulating apoptosis in a cell comprising modulating the activity of a pancortin polypeptide. In particular embodiments, modulating apoptosis in a cell further comprises modulating the activity of a pablo polypeptide.

In another embodiment, the present invention is directed to a method of modulating apoptosis in a cell comprising modulating the expression of a polynucleotide encoding a pancortin polypeptide. In particular embodiments, modulating apoptosis in a cell further comprises modulating the expression of a polynucleotide encoding a pablo polypeptide.

In yet another embodiment, the invention is directed to a method of treating a subject for a nervous system disorder comprising modulating the activity of a pancortin polypeptide and/or modulating the expression of a polynucleotide encoding a pancortin polypeptide.

In particular embodiments, the polynucleotide has at least one mutation selected from the group consisting of nucleotide deletion, nucleotide substitution and nucleotide insertion. In still another embodiment, the invention is directed to a method for assaying the effects of test compounds on the activity of a pancortin polypeptide comprising the steps of providing recombinant cells comprising a polynucleotide expressing a pancortin polypeptide, contacting the cells with a test compound and determining the effects of the test compound on the activity of the pancortin in the presence and absence of the test compound. In one particular embodiment, the polynucleotide has at least one mutation selected from the group consisting of nucleotide deletion, nucleotide substitution and nucleotide insertion. In another particular embodiment, the recombinant cell may further comprise a polynucleotide expressing a pablo polypeptide. In other embodiments, a method for assaying the effects of test compounds on the binding interaction of pancortin and pablo polypeptides is provided comprising the steps of providing yeast cells for a yeast two-hybrid system comprising a pancortin polypeptide and a pablo polypeptide, contacting the cells with a test compound and determining the effect of the test compound on the binding interaction of the pancortin and pablo polypeptides in the presence and absence of the test compound.

In yet another embodiment, the invention is directed to a method for producing a pancortin polypeptide comprising an amino acid sequence of SEQ ID NO:2, SEQ ID NO:4, SEQ ID NO:6, SEQ ID NO:8, a variant thereof or a fragment thereof, comprising transfecting, transforming or infecting a recombinant host cell with an expression vector comprising a polynucleotide comprising a nucleotide sequence of SEQ ID NO:1, SEQ ID NO:3, SEQ ID NO:5, SEQ ID NO:7, or a degenerate variant thereof, culturing the host cell under conditions sufficient for the production of the polypeptide and isolating the polypeptide from the culture.

In yet another embodiment, the invention is directed to a method for the treatment of a subject in need of reduced pancortin activity comprising administering to the subject a therapeutically effective amount of a pancortin antagonist and/or administering to the subject a polynucleotide encoding an antisense RNA polynucleotide comprising a nucleotide sequence that is a complement to a nucleotide sequence of SEQ ID NO:1, SEQ ID NO:3, SEQ ID NO:5, SEQ ID NO:7, a degenerate variant thereof or a fragment thereof.

In still other embodiments, a method for the diagnosis of a disease or the susceptibility to a disease in a subject related to the expression or activity of a pancortin polypeptide in the subject comprising determining the presence or absence of a mutation in a polynucleotide encoding a pancortin polypeptide comprising an amino acid sequence of SEQ ID NO:2, SEQ ID NO:4, SEQ ID NO:6, SEQ ID NO:8, or a fragment thereof and/or assaying for the presence of pancortin expression in a sampled derived from the subject, wherein the pancortin expressed is a polynucleotide encoding a pancortin polypeptide comprising an amino acid sequence of SEQ ID NO:2, SEQ ID NO:4, SEQ ID NO:6, SEQ ID NO:8, or a fragment thereof.

In particular embodiments, the invention is directed to a composition for treating a hyperproliferative disease comprising a pancortin polypeptide comprising the amino acid sequence of SEQ ID NO:2, SEQ ID NO:4, SEQ ID NO:6, or SEQ ID NO:8 and a pablo polypeptide comprising the amino acid sequence of SEQ ID NO:10. In a preferred embodiment, the hyperproliferative disease is selected from the group consisting of cancer, psoriasis, restenosis, atherosclerosis and fibrosis.

Other features and advantages of the invention will be apparent from the following detailed description, from the preferred embodiments thereof, and from the claims.

### BRIEF DESCRIPTION OF THE DRAWINGS

FIG. 1 is a schematic representation of the four pancortin mRNA protein coding sequences. Alternative splicing results in the pancortin 1, pancortin 2, pancortin 3 and pancortin 4 isoforms.

FIG. 2A shows the genomic organization of pancortin in mice, arranged in eight exons (black squares) located along 28 kb of DNA. The pancortin domain corresponding to each exon is indicated.

FIG. 2B is a schematic representation of alternative splicing resulting in four pancortin cDNAs. The open reading frame sizes contributed are A=66 bp, B=150 bp, M=306 bp, Y=6 bp, and Z=1002 bp.

FIG. 3 is a schematic showing the strategy for the targeting vector used in the generation of pancortin knockout mice. The strategy will yield a mouse that is a constitutive knockout for the Y exon, and can have the M2 exon inducibly knocked out by use of the CRE-LOX system.

### DETAILED DESCRIPTION OF THE INVENTION

Apoptosis occurs via a generally well accepted sequence of events that involves activation of a family of caspases. A variety of extracellular signals are known to trigger caspase activation and lead to apoptotic cell death. What is not well understood however are the steps and molecules that are involved in transmission and integration of apoptotic signaling.

It has previously been demonstrated that a protein identified as pro-apoptotic Bcl-X_{L} binding protein,” hereinafter referred to as Pablo, interacts with the anti-apoptotic BCl-X_{L} protein, modulates apoptosis in cells, and is neuron specific (U.S. patent application Ser. No. 09/425,501, filed Oct. 22, 1999, specifically incorporated by reference herein in its entirety). The present invention has identified proteins which interact with Pablo. Specifically, the present invention has identified a protein family, termed pancortins, which bind, interact or associate with Pablo and “mediate” Pablo induced apoptosis.

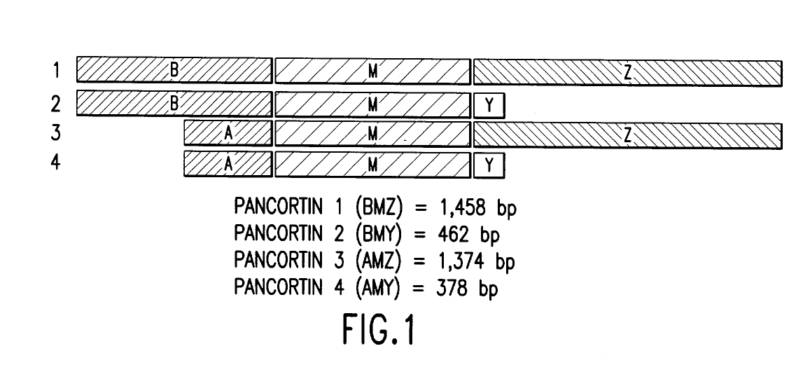

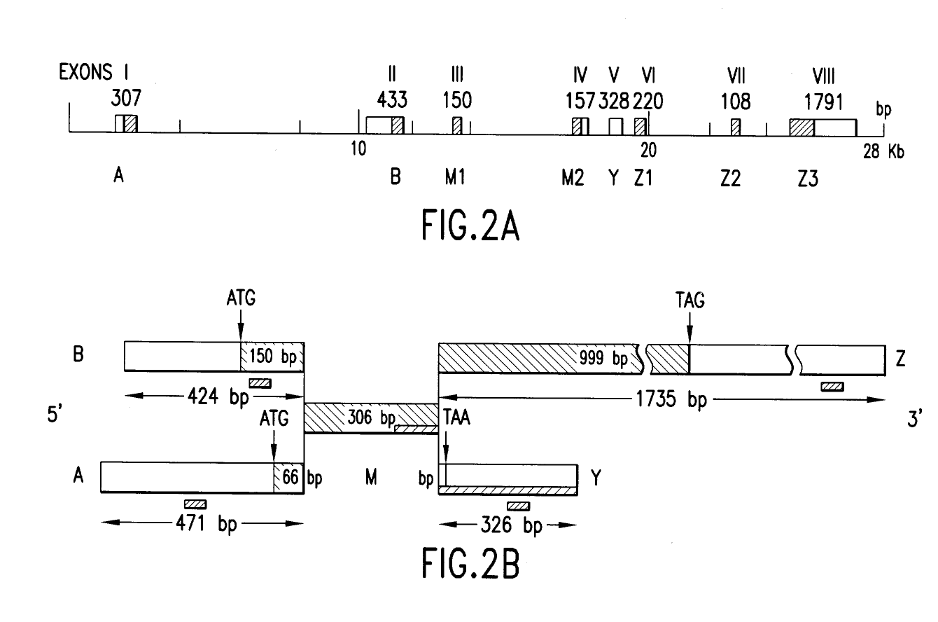

Pancortins represent a family of brain-specific glycoproteins, which were initially identified based on cloning of brain-specific transcripts (Danielson et al., 1994). Differentially processed pancortin transcripts are expressed in the rat brain in a developmental and region-specific manner (Nagano et al., 1998). Four pancortin proteins arise from the usage of two 5′ exons (A and B with independent promoters) along with distinct 3′ exons that encode two different C-termini of the proteins (termini Y and Z). Matrixing of all combinations results in 4 species of mRNA and proteins that share a middle region (M) (see FIG. 1 and FIG. 2B).

Pancortins 3 and 4 are the dominant forms during development and may be secreted, while pancortins 1 and 2 predominate during adulthood (Nagano et al., 2000). It is demonstrated in the present invention that the pancortin family of proteins are localized to the endoplasmic reticulum (ER) (Nagano et al., 1998). Thus, if pancortins 3 and 4 are truly secreted proteins, then their association with the ER is expected as part of the secretory pathway. The present invention shows that pancortin 2 is a non-secreted, ER resident or associated protein. There are numerous literature reports citing the importance of the endoplasmic reticulum, in apoptosis in general, and in neuronal apoptosis in particular. Pancortin 2 and pancortin 4 were observed in the present invention to bind to Pablo in yeast two-hybrid assays, whereas pancortin 1 and 3 do not bind to Pablo (data not shown). The failure of pancortins 1 and 3 to bind to Pablo suggests that either the Z domain sterically hinders such an interaction or the glycine residue, which is the Y domain, is of critical importance in the binding. However, of the four pancortin isoforms, pancortin 2 appears to functionally interact in vivo with Pablo. Transfection of pancortin 2 leads to increased cell death in cultured neuronal cells, presumably by interacting with endogenous cellular factors. Co-transfections of pancortin 2 with Pablo decreases viability of non-neuronal cells while transfection of either alone has minimal effect, indicating that pancortin 2 is a partner for Pablo and mediates Pablo-induced apoptosis in the central nervous system (CNS). No such synergistic apoptotic activity in non-neuronal cells is observed with co-transfection of Pablo and pancortins 1, 3, or 4. The discrepancy seen with pancortin 4, that is, it binds to Pablo in the yeast two hybrid, but has no pro-apoptotic consequences in mammalian apoptosis assays, has at least three possible explanations. Firstly, the intracellular localization/targeting of pancortin 4 may keep it physically separated from Pablo, thus precluding binding. Secondly, pancortin 4 may bind to Pablo in vivo in an anti-apoptotic manner. Thirdly, pancortin 4 binding to Pablo in vivo may play no role in apoptosis whatsoever.

The nucleic acid sequence of pancortin 1, pancortin 2, pancortin 3 and pancortin 4 are SEQ ID NO:1, SEQ ID NO:3, SEQ ID NO:5, and SEQ ID NO:7, respectively. The amino acid sequence of pancortin 1, pancortin 2, pancortin 3 and pancortin 4 are SEQ ID NO:2, SEQ ID NO:4, SEQ ID NO:6, and SEQ ID NO:8, respectively. The nucleic acid sequence of Pablo is SEQ ID NO:9, which encodes a Pablo protein of SEQ ID NO:10.

The polynucleotide sequence of SEQ ID NO:11 encodes a Pablo polypeptide fragment of having an amino acid sequence SEQ ID NO:12. This Pablo fragment comprises the pancortin binding domain of the full length Pablo polypeptide (i.e., SEQ ID NO:10). Similarly, SEQ ID NO:13 encodes a pancortin 2 polypeptide fragment of SEQ ID NO:14, which comprises the Pablo binding domain of the full length pancortin 2 polypeptide (i.e., SEQ ID NO:4). Further, nucleotides 280-459 of pancortin 2 (SEQ ID NO:3) are homologous to nucleotides 196-375 of pancortin 4 (SEQ ID NO:7). Nucleotides 280-456 of pancortin 2 (SEQ ID NO:3) are homologous to nucleotides 280-456 of pancortin 1 (SEQ ID NO:1) and are homologous to nucleotides 196-372 of pancortin 3 (SEQ ID NO:5).

Thus, the present invention relates to newly identified polypeptide interactions, comprising a neuron-specific pancortin polypeptide and a neuron-specific pro-apoptotic Pablo polypeptide, the use of such polypeptides, the modulation of such polypeptides, as well as the production of such polypeptides. The invention also relates to identifying compounds which may be agonists, antagonists and/or inhibitors of the pancortin-Pablo interaction, and therefore potentially useful in preventing, ameliorating or correcting dysfunctions or diseases related to cellular apoptosis.

Compositions and methods for use of the polynucleotides, polypeptides, antibodies, expression vectors, host cells and transgenic animals of the present invention are discussed in the following sections.

A. Isolated Polynucleotides that Encode Pancortin and Pablo Polypeptides

Isolated and purified pancortin and Pablo polynucleotides of the present invention are contemplated for use in the production of pancortin and Pablo polypeptides and fragments thereof. In particular embodiments, the pancortin and Pablo polypeptides and fragments thereof are used in methods for assaying the effects of test compounds on the activity of pancortin-Pablo interactions, methods for assaying the effects of test compounds on the activity or interactions of pancortin and Pablo comprised in transgenic animals encoding pancortin and/or pancortin-Pablo, methods for diagnosis and treatment of diseases related to the activity of pancortin and/or pancortin-Pablo and methods for modulating pancortin and/or pancortin-Pablo activity. In other embodiments, antibodies are provided specific for pancortin polypeptides and fragments thereof, pancortin-Pablo polypeptide dimers and fragments thereof, transgenic animals comprising functional disruptions in a polynucleotide encoding a pancortin polypeptide, recombinant expression vectors encoding pancortin and/or pancortin-Pablo polypeptides, and host cells comprising these vectors. As defined herein, the term “pancortin-Pablo” includes the presence of both a pancortin and a Pablo polypeptide.

Thus, in one aspect, the present invention provides isolated and purified polynucleotides that encode pancortin and/or pancortin-Pablo polypeptides. In particular embodiments, a polynucleotide of the present invention is a DNA molecule. In a preferred embodiment, a polynucleotide of the present invention encodes an isolated human pancortin polypeptide comprising the amino acid sequence of SEQ ID NO:2, SEQ ID NO:4, SEQ ID NO:6, SEQ ID NO:8, a variant thereof or a fragment thereof. In particular embodiments, an isolated polynucleotide encoding a pancortin polypeptide comprises the nucleotide sequence of SEQ ID NO:1, SEQ ID NO:3, SEQ ID NO:5, SEQ ID NO:7, a degenerate variant thereof, or a fragment thereof. In certain embodiments, an isolated Pablo polypeptide is further provided, wherein the Pablo polypeptide is encoded by a polynucleotide comprising the nucleotide sequence of SEQ ID NO:9.

As used herein, the term “polynucleotide” means a sequence of nucleotides connected by phosphodiester linkages. Polynucleotides are presented herein in the direction from the 5′ to the 3′ direction. A polynucleotide of the present invention can comprise from about 40 to about several hundred thousand base pairs. Preferably, a polynucleotide comprises from about 10 to about 3,000 base pairs. Preferred lengths of particular polynucleotides are set forth hereinafter.

A polynucleotide of the present invention can be a deoxyribonucleic acid (DNA) molecule, a ribonucleic acid (RNA) molecule, or analogs of the DNA or RNA generated using nucleotide analogs. The nucleic acid molecule can be single-stranded or double-stranded, but preferably is double-stranded DNA. Where a polynucleotide is a DNA molecule, that molecule can be a gene, a cDNA molecule or a genomic DNA molecule. Nucleotide bases are indicated herein by a single letter code: adenine (A), guanine (G), thymine (T), cytosine (C), inosine (I) and uracil (U).

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed herein.

Preferably, an “isolated” polynucleotide is free of sequences which naturally flank the nucleic acid (i.e., sequences located at the 5′ and 3′ ends of the nucleic acid) in the genomic DNA of the organism from which the nucleic acid is derived. For example, in various embodiments, the isolated pancortin and/or pancortin-Pablo nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., neuronal or placental). However, the pancortin nucleic acid molecule can be fused to other protein encoding or regulatory sequences and still be considered isolated.

Polynucleotides of the present invention may be obtained, using standard cloning and screening techniques, from a cDNA library derived from mRNA from human cells or from genomic DNA. Polynucleotides of the invention can also be synthesized using well known and commercially available techniques.

The invention further encompasses nucleic acid molecules that differ from the nucleotide sequences of SEQ ID NO:1, SEQ ID NO:3, SEQ ID NO:5 or SEQ ID NO:7, encoding a human pancortin polypeptide, due to degeneracy of the genetic code and thus encode the same pancortin polypeptide as that encoded by the nucleotide sequence shown in SEQ ID NO:1, SEQ ID NO:3, SEQ ID NO:5 or SEQ ID NO:7.

In another preferred embodiment, an isolated polynucleotide of the invention comprises a nucleic acid molecule which is a complement of the nucleotide sequence shown in SEQ ID NO:1, SEQ ID NO:3, SEQ ID NO:5, SEQ ID NO:7, or a fragment of these nucleotide sequences. A nucleic acid molecule which is complementary to the nucleotide sequence shown in SEQ ID NO:1, SEQ ID NO:3, SEQ ID NO:5 or SEQ ID NO:7 is one which is sufficiently complementary to the nucleotide sequence SEQ ID NO:1, SEQ ID NO:3, SEQ ID NO:5 or SEQ ID NO:7, such that it can hybridize to the nucleotide sequence shown in SEQ ID NO:1, SEQ ID NO:3, SEQ ID NO:5 or SEQ ID NO:7, thereby forming a stable duplex.

Orthologues and allelic variants of the human pancortin polynucleotides can readily be identified using methods well known in the art. Allelic variants and orthologues of pancortins will comprise a nucleotide sequence that is typically at least about 70-75%, more typically at least about 80-85%, and most typically at least about 90-95% or more homologous to the nucleotide sequence shown in SEQ ID NO:1, SEQ ID NO:3, SEQ ID NO:5 or SEQ ID NO:7, or a fragment of these nucleotide sequences. Such nucleic acid molecules can readily be identified as being able to hybridize, preferably under stringent conditions, to the nucleotide sequence shown in SEQ ID NO:1, SEQ ID NO:3, SEQ ID NO:5 or SEQ ID NO:7, or a fragment of these nucleotide sequences.

When the polynucleotides of the invention are used for the recombinant production of pancortin and/or pancortin-Pablo polypeptides of the present invention, the polynucleotide may include the coding sequence for the mature polypeptide, by itself, or the coding sequence for the mature polypeptide in a reading frame with other coding sequences, such as those encoding a leader or secretory sequence, a pre-, or pro- or prepro-polypeptide sequence, or other fusion peptide portions. For example, a marker sequence which facilitates purification of the fused polypeptide can be encoded (see Gentz et al., 1989, incorporated herein by reference in its entirety). The polynucleotide may also contain non-coding 5′ and 3′ sequences, such as transcribed, non-translated sequences, splicing and polyadenylation signals, ribosome binding sites and sequences that stabilize mRNA.

In addition to the pancortin nucleotide sequences shown in SEQ ID NO:1, SEQ ID NO:3, SEQ ID NO:5 and SEQ ID NO:7, it will be appreciated by those skilled in the art that DNA sequence polymorphisms that lead to changes in the amino acid sequences of pancortin polypeptides may exist within a population (e.g., the human population). Such genetic polymorphism in the pancortin gene or polynucleotide may exist among individuals within a population due to natural allelic variation. As used herein, the terms “gene” and “recombinant gene” refer to polynucleotides comprising an open reading frame encoding a pancortin polypeptide, preferably a human pancortin polypeptide. Such natural allelic variations can typically result in 1-5% variance in the nucleotide sequence of the pancortin polynucleotide. Any and all such nucleotide variations and resulting amino acid polymorphisms in a pancortin polynucleotide that are the result of natural allelic variation are intended to be within the scope of the invention. Such allelic variation includes both active allelic variants as well as non-active or reduced activity allelic variants, the latter two types typically giving rise to a pathological disorder.

Moreover, nucleic acid molecules encoding pancortin polypeptides from other species, and thus which have a nucleotide sequence which differs from the human sequence of SEQ ID NO:1, SEQ ID NO:3, SEQ ID NO:5 or SEQ ID NO:7, are intended to be within the scope of the invention. Polynucleotides corresponding to natural allelic variants and non-human orthologues of the human pancortin cDNAs of the invention can be isolated based on their homology to the human pancortin polynucleotides disclosed herein using the human cDNA, or a fragment thereof, as a hybridization probe according to standard hybridization techniques under stringent hybridization conditions.

Thus, a polynucleotide encoding a polypeptide of the present invention, including homologs and orthologs from species other than human, may be obtained by a process which comprises the steps of screening an appropriate library under stringent hybridization conditions with a labeled probe having the sequence of SEQ ID NO:1, SEQ ID NO:3, SEQ ID NO:5 or SEQ ID NO:7, or a fragment thereof; and isolating full-length cDNA and genomic clones containing the polynucleotide sequence. Such hybridization techniques are well known to the skilled artisan. The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low “processivity” (a measure of the ability of the enzyme to remain attached to the template during the polymerization reaction), failing to complete a DNA copy of the mRNA template during 1 st strand cDNA synthesis.

Thus, in certain embodiments, the polynucleotide sequence information provided by the present invention allows for the preparation of relatively short DNA (or RNA) oligonucleotide sequences having the ability to specifically hybridize to gene sequences of the selected polynucleotides disclosed herein. The term “oligonucleotide” as used herein is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence, e.g., a sequence such as that shown in SEQ ID NO:1, SEQ ID NO:3, SEQ ID NO:5 or SEQ ID NO:7. The ability of such nucleic acid probes to specifically hybridize to a polynucleotide encoding a pancortin polypeptide lends them particular utility in a variety of embodiments. Most importantly, the probes can be used in a variety of assays for detecting the presence of complementary sequences in a given sample.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of a gene or polynucleotide that encodes a pancortin polypeptide from mammalian cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

Polynucleotides which are identical or sufficiently identical to a nucleotide sequence contained in SEQ ID NO:1, SEQ ID NO:3, SEQ ID NO:5, SEQ ID NO:7, or a fragment thereof, may be used as hybridization probes for cDNA and genomnic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to SEQ ID NO:1, SEQ ID NO:3, SEQ ID NO:5, SEQ ID NO:7, or a fragment thereof. Typically these nucleotide sequences are from at least about 70% identical to at least about 95% identical to that of the reference polynucleotide sequence. The probes or primers will generally comprise at least 15 nucleotides, preferably, at least 30 nucleotides and may have at least 50 nucleotides. Particularly preferred probes will have between 30 and 50 nucleotides.

There are several methods available, and well known to those skilled in the art, to obtain full-length cDNAs, or extend short cDNAs, for example those based on the method of Rapid Amplification of cDNA ends (RACE) (see, Frohman et al., 1988). Recent modifications of the technique, exemplified by the Marathon™ technology (Clontech Laboratories Inc.) have significantly simplified the search for longer cDNAs. In the Marathon™ technology, cDNAs have been prepared from mRNA extracted from a chosen tissue and an “adaptor” sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

To provide certain of the advantages in accordance with the present invention, a preferred nucleic acid sequence employed for hybridization studies or assays includes probe molecules that are complementary to at least a 10 to 70 or more long nucleotide stretch of a polynucleotide that encodes a pancortin polypeptide, such as that shown in SEQ ID NO:2, SEQ ID NO:4, SEQ ID NO:6 or SEQ ID NO:8. A size of at least 10 nucleotides in length helps to ensure that the fragment will be of sufficient length to form a duplex molecule that is both stable and selective. Molecules having complementary sequences over stretches greater than 10 bases in length are generally preferred, in order to increase stability and selectivity of the hybrid, and thereby improve the quality and degree of specific hybrid molecules obtained. One will generally prefer to design nucleic acid molecules having gene-complementary stretches of 25 to 40 nucleotides, 55 to 70 nucleotides, or even longer where desired. Such fragments can be readily prepared by, for example, directly synthesizing the fragment by chemical means, by application of nucleic acid reproduction technology, such as the PCR technology (U.S. Pat. No. 4,683,202, incorporated by reference herein in its entirety) or by excising selected DNA fragments from recombinant plasmids containing appropriate inserts and suitable restriction enzyme sites.

In another aspect, the present invention contemplates an isolated and purified polynucleotide comprising a base sequence that is identical or complementary to a segment of at least 10 contiguous bases of SEQ ID NO:1, SEQ ID NO:3, SEQ ID NO:5 or SEQ ID NO:7, wherein the polynucleotide hybridizes to a polynucleotide that encodes a pancortin polypeptide. Preferably, the isolated and purified polynucleotide comprises a base sequence that is identical or complementary to a segment of at least 25 to 70 contiguous bases of SEQ ID NO:1, SEQ ID NO:3, SEQ ID NO:5 or SEQ ID NO:7. For example, the polynucleotide of the invention can comprise a segment of bases identical or complementary to 40 or 55 contiguous bases of the disclosed nucleotide sequences.

Accordingly, a polynucleotide probe molecule of the invention can be used for its ability to selectively form duplex molecules with complementary stretches of the gene. Depending on the application envisioned, one will desire to employ varying conditions of hybridization to achieve a varying degree of selectivity of the probe toward the target sequence. For applications requiring a high degree of selectivity, one will typically desire to employ relatively stringent conditions to form the hybrids (see Table 1).

Of course, for some applications, for example where one desires to prepare mutants employing a mutant primer strand hybridized to an underlying template, or where one seeks to isolate a pancortin polynucleotide coding sequence from other cells, functional equivalents, or the like, less stringent hybridization conditions are typically needed to allow formation of the heteroduplex. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

The present invention also includes polynucleotides capable of hybridizing under reduced stringency conditions, more preferably stringent conditions, and most preferably highly stringent conditions, to polynucleotides described herein. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| Strin- | | | | |
|---|---|---|---|---|
| gency | | Hybrid | Hybridization | Wash |
| Con- | Polynucleotide | Length | Temperature and | Temperature |
| dition | Hybrid | (bp)^{I} | Buffer^{H} | and Buffer^{H} |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}; 1xSSC | T_{B}; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}; 1xSSC | T_{D}; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}; 1xSSC | T_{f}; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}; 4xSSC | T_{H}; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}; 4xSSC | T_{J}; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}; 2xSSC | T_{L}; 2xSSC |
| M | DNA:DNA | >50 | 50° C.; 4xSSC -or- | 50° C.; 2xSSC |
| | | | 40° C.; 6xSSC, 50% | |
| | | | formamide | |
| N | DNA:DNA | <50 | T_{N}; 6xSSC | T_{N}; 6xSSC |
| O | DNA:RNA | >50 | 55° C.; 4xSSC -or- | 55° C.; 2xSSC |
| | | | 42° C.; 6xSSC, 50% | |
| | | | formamide | |
| P | DNA:RNA | <50 | T_{P}; 6xSSC | T_{P}; 6xSSC |
| Q | RNA:RNA | >50 | 60° C.; 4xSSC -or- | 60° C.; 2xSSC |
| | | | 45° C.; 6xSSC, 50% | |
| | | | formamide | |
| R | RNA:RNA | <50 | T_{R}; 4xSSC | T_{R}; 4xSSC |

(bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.

Buffer^{H}: SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete.

T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M).

Additional examples of stringency conditions for polynucleotide hybridization are provided in Sambrook et al., 1989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, eds., John Wiley & Sons, Inc., sections 2.10 and 6.3-6.4, incorporated herein by reference.

In addition to the nucleic acid molecules encoding pancortin polypeptides described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire pancortin coding strand, or to only a fragment thereof. In one embodiment, an antisense nucleic acid molecule is antisense to a “coding region” of the coding strand of a nucleotide sequence encoding a pancortin polypeptide.

The term “coding region” refers to the region of the nucleotide sequence comprising codons which are translated into amino acid residues, e.g., the entire coding region of SEQ ID NO:1, SEQ ID NO:3, SEQ ID NO:5 or SEQ ID NO:7. In another embodiment, the antisense nucleic acid molecule is antisense to a “noncoding region” of the coding strand of a nucleotide sequence encoding a pancortin polypeptide. The term “noncoding region” refers to 5′ and 3′ sequences which flank the coding region that are not translated into amino acids (i.e., also referred to as 5′ and 3′ untranslated regions).

Given the coding strand sequence encoding the pancortin polypeptide disclosed herein (e.g., SEQ ID NO:1, SEQ ID NO:3, SEQ ID NO:5 or SEQ ID NO:7), antisense nucleic acids of the invention can be designed according to the rules of Watson and Crick base pairing. The antisense nucleic acid molecule can be complementary to the entire coding region of pancortin mRNA, but more preferably is an oligonucleotide which is antisense to only a fragment of the coding or noncoding region of pancortin mRNA. For example, the antisense oligonucleotide can be complementary to the region surrounding the translation start site of pancortin mRNA.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. In addition, backbone modifications such as peptide nucleic acids (PNAs) are contemplated for use in the invention (see U.S. Pat. No. 6,201,103).

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a pancortin polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual γ-units, the strands run parallel to each other (Gaultier et al., 1987). The antisense nucleic acid molecule can also comprise a 2′-o-methylribonucleotide (Inoue et al., 1987(a)) or a chimeric RNA-DNA analogue (Inoue et al., 1987(b)).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach, 1988)) can be used to catalytically cleave pancortin mRNA transcripts to thereby inhibit translation of pancortin mRNA. A ribozyme having specificity for a pancortin-encoding nucleic acid can be designed based upon the nucleotide sequence of a pancortin cDNA disclosed herein (i.e., SEQ ID NO:1, SEQ ID NO:3, SEQ ID NO:5 or SEQ ID NO:7). For example, a derivative of a Tetrahymena L-19 IVS RNA can be constructed in which the nucleotide sequence of the active site is complementary to the nucleotide sequence to be cleaved in a pancortin-encoding mRNA. See, e.g., Cech et al. U.S. Pat. No. 4,987,071 and Cech et al. U.S. Pat. No. 5,116,742, both of which are incorporated by reference herein in their entirety. Alternatively, pancortin mRNA can be used to select a catalytic RNA having a specific ribonuclease activity from a pool of RNA molecules. See, e.g., Bartel and Szostak, 1993).

Alternatively, pancortin gene expression can be inhibited by targeting nucleotide sequences complementary to the regulatory region of the pancortin gene (e.g., the pancortin gene promoter and/or enhancers) to form triple helical structures that prevent transcription of the pancortin gene in target cells. See generally, Helene, 1991; Helene et al., 1992; and Maher, 1992).

Pancortin gene expression can also be inhibited using RNA interference (RNAi). This is a technique for post-transcriptional gene silencing (PTGS), in which target gene activity is specifically abolished with cognate double-stranded RNA (dsRNA). RNAi resembles in many aspects PTGS in plants and has been detected in many invertebrates including trypanosome, hydra, planaria, nematode and fruit fly (Drosophila melangnoster). It may be involved in the modulation of transposable element mobilization and antiviral state formation. RNAi in mammalian systems is disclosed in International Application No. WO 00/63364, which is incorporated by reference herein in its entirety. Basically, dsRNA of at least 600 nucleotides, homologous to the target (pancortin) is introduced into the cell and a sequence specific reduction in gene activity is observed.

B. Pancortin and Pablo Polypeptides

In particular embodiments, the present invention provides isolated and purified pancortin and/or pancortin-Pablo polypeptides and fragments thereof. Preferably, a pancortin and/or a pancortin-Pablo polypeptide of the invention is a recombinant polypeptide. In certain embodiments, a pancortin and/or a pancortin-Pablo polypeptide is produced by recombinant expression in a non-human cell. In certain embodiments, a pancortin polypeptide of the present invention comprises the amino acid sequence of SEQ ID NO:2, SEQ ID NO:4, SEQ ID NO:6, SEQ ID NO:8, a variant thereof or a fragment thereof. In other embodiments, the invention further provides a Pablo polypeptide comprising the amino acid sequence of SEQ ID NO:10, a variant thereof or a fragment thereof.

A pancortin polypeptide according to the present invention encompasses a polypeptide that comprises: 1) the amino acid sequence shown in SEQ ID NO:2, SEQ ID NO:4, SEQ ID NO:6 or SEQ ID NO:8; 2) functional and non-functional naturally occurring allelic variants of human pancortin polypeptides; 3) recombinantly produced variants of human pancortin polypeptides; and 4) pancortin polypeptides isolated from organisms other than humans (orthologues of human pancortin polypeptides.)

An allelic variant of human pancortin polypeptides according to the present invention encompasses 1) a polypeptide isolated from human cells or tissues; 2) a polypeptide encoded by the same genetic locus as that encoding the human pancortin polypeptide; and 3) a polypeptide that contains substantial homology to a human pancortin.

Allelic variants of human pancortin include both functional and non-functional pancortin polypeptides. Functional allelic variants are naturally occurring amino acid sequence variants of the human pancortin polypeptide that maintain the ability to bind a pancortin ligand (e.g., Pablo) and transduce a signal (e.g., modulate apoptosis) within a cell. Functional allelic variants will typically contain only a conservative substitution of one or more amino acids of SEQ ID NO:2, SEQ ID NO:4, SEQ ID NO:6 or SEQ ID NO:8 or a substitution, deletion or insertion of non-critical residues in non-critical regions of the polypeptide.

Non-functional allelic variants are naturally occurring amino acid sequence variants of human pancortin polypeptides that do not have the ability to either bind ligand and/or transduce a signal within a cell. Non-functional allelic variants will typically contain a non-conservative substitution, a deletion, or insertion or premature truncation of the amino acid sequence of SEQ ID NO:2, SEQ ID NO:4, SEQ ID NO:6 or SEQ ID NO:8 or a substitution, insertion or deletion in critical residues or critical regions.

The present invention further provides non-human orthologues of human pancortin polypeptides. Orthologues of human pancortin polypeptides are polypeptides that are isolated from non-human organisms and possess the same ligand binding and signaling capabilities as the human pancortin polypeptides. Orthologues of the human pancortin polypeptide can readily be identified as comprising an amino acid sequence that is substantially homologous to SEQ ID NO:2, SEQ ID NO:4, SEQ ID NO:6 or SEQ ID NO:8.

Modifications and changes can be made in the structure of a polypeptide of the present invention and still obtain a molecule having pancortin-like characteristics. For example, certain amino acids can be substituted for other amino acids in a sequence without appreciable loss of protein activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated reference herein in its entirety, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take a variety of the foregoing characteristics into consideration are well known to those of skill in the art and include: arginine and lysine; glutamate and aspartate; serine and threonine; glutamine and asparagine; and valine, leucine and isoleucine (see Table 2). The present invention thus contemplates functional or biological equivalents of human pancortin polypeptide as set forth above.

TABLE 2

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single stranded and double stranded form. Typically, site-directed mutagenesis in accordance herewith is performed by first obtaining a single-stranded vector which includes within its sequence a DNA sequence which encodes all, or a portion of, the pancortin polypeptide sequence selected. An oligonucleotide primer bearing the desired mutated sequence is prepared (e.g., synthetically). This primer is then annealed to the single-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

A pancortin polypeptide is a pancortin that participates in apoptotic signaling pathways within cells. As used herein, an apoptotic signaling pathway refers to the modulation (e.g., stimulated or inhibited) of a cellular function/activity upon the binding of a ligand to the pancortin or Pablo (pancortin or Pablo polypeptide). Examples of such functions include mobilization of intracellular molecules that participate in a signal transduction pathway, e.g., phosphatidylinositol 4,5-bisphosphate (PIP₂), inositol 1,4,5-triphosphate (IP₃) or adenylate cyclase; polarization of the plasma membrane; production or secretion of molecules; alteration in the structure of a cellular component; cell proliferation, e.g., synthesis of DNA; cell migration; cell differentiation; and cell survival. As the pancortin polypeptide identified is expressed substantially in the brain, examples of cells participating in a pancortin signaling pathway are contemplated in the present invention and include neural cells, e.g., peripheral nervous system and central nervous system cells such as brain cells, e.g., limbic system cells, hypothalamus cells, hippocampus cells, substantia nigra cells, cortex cells, brain stem cells, neocortex cells, basal ganglion cells, caudate putamen cells, olfactory tubercle cells, and superior colliculi cells.

Apoptosis is a major form of programmed cell death that is used to remove excess, damaged, or infected cells throughout life. It is important in normal cell development, and loss of control of the apoptotic program contributes to many diseases, including accumulation of unwanted cells through insufficient apoptosis (e.g. cancer) and cell loss as a result of excessive apoptosis (e.g. neurodegeneration). Cells undergoing apoptosis display characteristic morphological features. These include membrane blebbing and nuclear and cytoplasmic condensation, followed by fragmentation of the cell into membrane-bound apoptotic bodies that are rapidly phagocytized by macrophages without leakage of cellular contents. Fragmentation of genomic DNA into oligonucleosomal fragments as a result of nuclease activation is observed in apoptosis and is a widely accepted biochemical hallmark of apoptotic death. Regardless of the initiating insult and the ensuing upstream death signals generated, the execution phase of apoptosis normally involves the activation of caspases. Caspases are mammalian homologues of the C. elegans gene product Ced-3. Fourteen members of the mammalian caspase family have been identified and they are widely expressed in a variety of tissues and cell types. Caspase activation has been shown to contribute to cell death in the ischemic brain, ishcemic heart, and neuronal loss in chronic neurodegenerative diseases such as Alzheimer's disease and Huntington's disease.

The endoplasmic reticulum (ER) plays a key role in folding, modifying, and sorting newly synthesized proteins, maintaining intracellular Ca²⁺ homeostasis, and synthesizing lipids and sterols. When these processes are disturbed, at least three major ER stress-induced signaling pathways can be activated: 1) the unfolded protein response (UPR), 2) the ER-overload response (EOR) pathway, which leads to NF-κB activation and consequently production of cytokines, and 3) phosphorylation of a eukaryotic translation initiation factor (eIF-2a) which inhibits initiation of translation and thus, blocks protein synthesis.

Alterations of ER-mediated Ca²⁺ homeostasis are sufficient to induce apoptosis. For example, thapsigargin (an inhibitor of the ER Ca-ATPase) can induce apoptosis in neurons, and agents that suppress Ca²⁺ release from ER (e.g., dantrolene) can protect neurons against apoptosis. While the ability of agents that perturb ER Ca²⁺ homeostasis to induce neuronal apoptosis demonstrates that proper functioning of this organelle is necessary for neuronal survival, additional findings suggest that regulatory events occurring at the level of ER might control the cell death process. Bcl-xL is an anti-apoptotic protein that can prevent neuronal apoptosis in experimental models of developmental cell death and neurodegenerative disorders. It associates with the ER and mitochondrial membranes and stabilizes Ca²⁺ homeostasis and suppresses oxidative stress. In addition, agents which disrupt ER function (e.g., tunicamycin or brefeldin A), cause mitochondrial dysfunction and caspase activation.

ER stress or other apoptotic stimuli might also activate caspases at the ER surface and induce apoptosis. Murine caspase 12 is ubiquitously expressed in mouse tissues and resides predominantly on the outer ER membrane. Caspase 12 is activated by chemicals that induce ER stress (e.g., thapsigargin, A23187, brefeldin A, or tunicamycin), but not by insults that target the mitochondria. Localization of the Bcl-xL-BAP31-Procaspase 8 complex to the outer surface of the ER is another putative target of apoptotic signals, and the regulation of these sensors could be a consequence of a Pablo-Pancortin interaction.

A pancortin polypeptide of the present invention is understood to be any pancortin polypeptide comprising substantial sequence similarity, structural similarity and/or functional similarity to a pancortin polypeptide comprising the amino acid sequence selected from the group consisting of SEQ ID NO:2, SEQ ID NO:4, SEQ ID NO:6 or SEQ ID NO:8. In addition, a pancortin polypeptide of the invention is not limited to a particular source. Thus, the invention provides for the general detection and isolation of the genus of pancortin polypeptides from a variety of sources. Where there is a difference between species, identification of those differences is well within the skill of an artisan. Thus, the present invention contemplates a pancortin polypeptide from any mammal, wherein the preferred mammal is a human.

It is contemplated in the present invention, that a pancortin may advantageously be cleaved into fragments for use in further structural or functional analysis, or in the generation of reagents such as pancortin-related polypeptides and pancortin-specific antibodies. This can be accomplished by treating purified or unpurified pancortin with a peptidase such as endopolypeptidease glu-C (Boehringer, Indianapolis, Ind.). Treatment with CNBr is another method by which pancortin fragments may be produced from natural pancortin. Recombinant techniques also can be used to produce specific fragments of pancortin.

In addition, it also is contemplated that compounds sterically similar to a pancortin may be formulated to mimic the key portions of the peptide structure, called peptidomimetics. Mimetics are peptide-containing molecules which mimic elements of polypeptide secondary structure. See, for example, Johnson et al. (1993). The underlying rationale behind the use of peptide mimetics is that the peptide backbone of polypeptides exists chiefly to orient amino acid side chains in such a way as to facilitate molecular interactions, such as those of receptor and ligand.

Successful applications of the peptide mimetic concept have thus far focused on mimetics of β-turns within polypeptides. Likely β-turn structures within pancortin can be predicted by computer-based algorithms as discussed above. Once the component amino acids of the turn are determined, mimetics can be constructed to achieve a similar spatial orientation of the essential elements of the amino acid side chains, as discussed in Johnson et al. (1993).

“Fusion polypeptide” refers to a polypeptide encoded by two, often unrelated, fused genes or fragments thereof. For example, fusion polypeptides comprising various portions of constant region of immunoglobulin molecules together with another human polypeptide or part thereof have been described. In many cases, employing an immunoglobulin Fc region as a part of a fusion polypeptide is advantageous for use in therapy and diagnosis resulting in, for example, improved pharmacokinetic properties. On the other hand, for some uses it would be desirable to be able to delete the Fc part after the fusion polypeptide has been expressed, detected and purified.

C. Pancortin and Pancortin-Pablo Antibodies

In another embodiment, the present invention provides antibodies immunoreactive with a pancortin polypeptide. In other embodiments, the invention provides antibodies immunoreactive with pancortin-Pablo dimers. Preferably, the antibodies of the invention are monoclonal antibodies. Additionally, the pancortin polypeptides comprise the amino acid residue sequence of SEQ ID NO:2, SEQ ID NO:4, SEQ ID NO:6 or SEQ ID NO:8 and a Pablo polypeptide comprises the amino acid residue sequence of SEQ ID NO:10. Means for preparing and characterizing antibodies are well known in the art (see, e.g., Antibodies “A Laboratory Manual, E. Howell and D. Lane, Cold Spring Harbor Laboratory, 1988). In yet other embodiments, the present invention provides antibodies immunoreactive with pancortin polynucleotides.

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e.g., a polypeptide or polynucleotide) of the present invention with a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier polypeptide are well known in the art and include glutaraldehyde, m-maleimidobencoyl-N-hydroxysuccinimide ester, carbodiimide and bis-biazotized benzidine.

As is also well known in the art, immunogenicity to a particular immunogen can be enhanced by the use of non-specific stimulators of the immune response known as adjuvants. Exemplary and preferred adjuvants include complete Freund's adjuvant, incomplete Freund's adjuvants and aluminum hydroxide adjuvant.

The amount of immunogen used of the production of polyclonal antibodies varies inter alia, upon the nature of the immunogen as well as the animal used for immunization. A variety of routes can be used to administer the immunogen subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

In another aspect, the present invention contemplates a process of producing an antibody immunoreactive with a pancortin or a pancortin-Pablo polypeptide dimer comprising the steps of (a) transfecting recombinant host cells with a polynucleotide that encodes a pancortin or a pancortin-Pablo polypeptide; (b) culturing the host cells under conditions sufficient for expression of the polypeptide; (c) recovering the polypeptides; and (d) preparing the antibodies to the polypeptides. Preferably, the host cell is transfected with the polynucleotide of SEQ ID NO:1, SEQ ID NO:3, SEQ ID NO:5, SEQ ID NO:7 or SEQ ID NO:9. Even more preferably, the present invention provides antibodies prepared according to the process described above.

A monoclonal antibody of the present invention can be readily prepared through use of well-known techniques such as those exemplified in U.S. Pat. No. 4,196,265, herein incorporated by reference in its entirety. Typically, a technique involves first immunizing a suitable animal with a selected antigen (e.g., a polypeptide or polynucleotide of the present invention) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, e.g., by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptide. The selected clones can then be propagated indefinitely to provide the monoclonal antibody.

By way of specific example, to produce an antibody of the present invention, mice are injected intraperitoneally with about 1-200 μg of an antigen comprising a polypeptide of the present invention. B lymphocyte cells are stimulated to grow by injecting the antigen in association with an adjuvant, such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the process of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells, known as myeloma cells, are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produce a single antibody. These cells are then screened for the production of the specific antibody immunoreactive with an antigen/polypeptide of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

By use of a monoclonal antibody of the present invention, specific, polypeptides and polynucleotide of the invention can be recognized as antigens, and thus identified. Once identified, those polypeptides and polynucleotides can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

Additionally, examples of methods and reagents particularly amenable for use in generating and screening antibody display library can be found in, for example, U.S. Pat. No. 5,223,409; International Application No. WO 92/18619; International Application No. WO 91/17271; International Application No. WO 92/20791; International Application No. WO 92/15679; International Application No. WO 93/01288; International Application No. WO 92/01047; International Application No. WO 92/09690; International Application No. WO 90/02809.

Additionally, recombinant anti-pancortin and/or anti-pancortin-Pablo antibodies, such as chimeric and humanized monoclonal antibodies, comprising both human and non-human fragments, which can be made using standard recombinant DNA techniques, are within the scope of the invention. Such chimeric and humanized monoclonal antibodies can be produced by recombinant DNA techniques known in the art, for example using methods described in U.S. Pat. No. 6,054,297; U.S. Pat. No. 4,816,567; European Application Nos. EP 184,187; EP 125,023; EP 171,496; EP 173,494; and International Application No. WO 86/01533.

Anti-pancortin or anti-pancortin-Pablo antibodies (e.g., monoclonal antibody) can be used to isolate pancortin or pancortin-Pablo polypeptide dimers, respectively, by standard techniques, such as affinity chromatography or immunoprecipitation. An anti-pancortin or anti-pancortin-Pablo antibody can facilitate the purification of a natural pancortin or pancortin-Pablo polypeptides from cells and recombinantly produced pancortin or pancortin-Pablo polypeptide expressed in host cells. Moreover, an anti-pancortin or anti-pancortin-Pablo antibody can be used to detect pancortin polypeptides or pancortin-Pablo polypeptide dimers (e.g., in a cellular lysate or cell supernatant) in order to evaluate the abundance and pattern of expression of the pancortin or pancortin-Pablo polypeptide. The detection of circulating fragments of a pancortin or pancortin-Pablo polypeptide can be used to identify pancortin or pancortin-Pablo polypeptide turnover in a subject. Anti-pancortin or anti-pancortin-Pablo antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ¹⁵S or ³H.

D. Vectors, Host Cells and Recombinant Pancortin and Pablo Polypeptides

In an alternate embodiment, the present invention provides expression vectors comprising polynucleotides that encode pancortin polypeptides or pancortin-Pablo dimers, or fragments thereof. Preferably, the expression vectors of the present invention comprise polynucleotides that encode polypeptides comprising the amino acid residue sequence of SEQ ID NO:2, SEQ ID NO:4, SEQ ID NO:6, SEQ ID NO:8 or SEQ ID NO:10. More preferably, the expression vectors of the present invention comprise polynucleotides comprising the nucleotide base sequence of SEQ ID NO:1, SEQ ID:3, SEQ ID NO:5, SEQ ID:7 or SEQ ID NO:9. Even more preferably, the expression vectors of the invention comprise polynucleotides operatively linked to an enhancer-promoter. In certain embodiments, the expression vectors of the invention comprise polynucleotides operatively linked to a prokaryotic promoter. Alternatively, the expression vectors of the present invention comprise polynucleotides operatively linked to an enhancer-promoter that is a eukaryotic promoter, and the expression vectors further comprise a polyadenylation signal that is positioned 3′ of the carboxy-terminal amino acid and within a transcriptional unit of the encoded polypeptide.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson,1988), pMAL (New England Biolabs, Beverly; Mass.) and pRIT5 (Pharmacia, Piscataway, N.J.) which fuse glutathione S-transferase (GST), maltose E binding protein, or protein A, respectively, to the target recombinant protein.

In one embodiment, the coding sequence of the pancortin or Pablo gene is cloned into a pGEX expression vector to create a vector encoding a fusion protein comprising, from the N-terminus to the C-terminus, GST-thrombin cleavage site-pancortin or -Pablo polypeptide. The fusion protein can be purified by affinity chromatography using glutathione-agarose resin. Recombinant pancortin or Pablo polypeptide unfused to GST can be recovered by cleavage of the fusion protein with thrombin.

Examples of suitable inducible non-fusion E. coli expression vectors include pTrc (Amann et al., 1988) and pET lid (Studier et al., 1990). Target gene expression from the pTrc vector relies on host RNA polymerase transcription from a hybrid trp-lac fusion promoter. Target gene expression from the pET lid vector relies on transcription from a T7 gn10-lac fusion promoter mediated by a coexpressed viral RNA polymerase J7 gnl. This viral polymerase is supplied by host strains BL21 (DE3) or HMS I 74(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention can be carried out by standard DNA mutagenesis or synthesis techniques.

In another embodiment, the pancortin or Pablo polynucleotide expression vector is a yeast expression vector. Examples of vectors for expression in yeast S. cerivisae include pYepSec I (Baldari, et al., 1987), pMFa (Kurjan and Herskowitz, 1982), pJRY88 (Schultz et al., 1987), and pYES2 (Invitrogen Corporation, San Diego, Calif.).

Alternatively, a pancortin or pancortin-Pablo polynucleotide can be expressed in insect cells using, for example, baculovirus expression vectors. Baculovirus vectors available for expression of proteins in cultured insect cells (e.g., Sf9 cells) include the pAc series (Smith et al., 1983) and the pVL series (Lucklow and Summers, 1989).

In yet another embodiment, a polynucleotide of the invention is expressed in mammalian cells using a mammalian expression vector. Examples of mammalian expression vectors include pCDM8 (Seed, 1987), pCDNA3-1 (Invitrogen) and pMT2PC (Kaufman et al., 1987). When used in mammalian cells, the expression vector's control functions are often provided by viral regulatory elements.

For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., “Molecular Cloning: A Laboratory Manual” 2nd, ed, Cold Spring Harbor Laboratory, Cold Spring Harbor Laboratory Press, Cold Spring Harbor, N.Y., 1989, incorporated by reference herein in its entirety.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987), lymphoid-specific promoters (Calame and Eaton, 1988), in particular promoters of T cell receptors (Winoto and Baltimore, 1989) and immunoglobulins (Banerji et al., 1983, Queen and Baltimore, 1983), neuron-specific promoters (e.g., the neurofilament promoter; Byrne and Ruddle, 1989), pancreas-specific promoters (Edlund et al., 1985), and mammary gland-specific promoters (e.g., milk whey promoter; U.S. Pat. No. 4,873,316 and European Application No. EP 264,166). Developmentally-regulated promoters are also encompassed, for example the murine hox promoters (Kessel and Gruss, 1990) and the α-fetoprotein promoter (Campes and Tilghman, 1989).

The invention further provides a recombinant expression vector comprising a DNA molecule encoding a pancortin or polypeptide cloned into the expression vector in an antisense orientation. That is, the DNA molecule is operatively linked to a regulatory sequence in a manner which allows for expression (by transcription of the DNA molecule) of an RNA molecule which is antisense to pancortin or Pablo mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, pancortin or Pablo polypeptide can be expressed in bacterial cells such as E coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation, infection or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (“Molecular Cloning: A Laboratory Manual” 2nd. Ed. Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the pancortin or Pablo polypeptide or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) pancortin or Pablo polypeptides. Accordingly, the invention further provides methods for producing pancortin or Pablo polypeptides using the host cells of the invention. In one embodiment, the method comprises culturing the host cell of invention (into which a recombinant expression vector encoding a pancortin or Pablo polypeptide has been introduced) in a suitable medium until the pancortin or Pablo polypeptide is produced. In another embodiment, the method further comprises isolating the Pancortin or Pablo polypeptide from the medium or the host cell.

A promoter is a region of a DNA molecule typically within about 100 nucleotide pairs in front of (upstream of) the point at which transcription begins (i.e., a transcription start site). That region typically contains several types of DNA sequence elements that are located in relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer-promoter that drives expression in a cell to be transfected. By employing an enhancer-promoter with well-known properties, the level and pattern of gene product expression can be optimized.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in an adenovirus vector construct of the present invention comprises a polyadenylation signal of SV40 or the protamine gene.

An expression vector comprises a polynucleotide that encodes a pancortin or Pablo polypeptide. Such a polypeptide is meant to include a sequence of nucleotide bases encoding a pancortin or Pablo polypeptide sufficient in length to distinguish said segment from a polynucleotide segment encoding a non-pancortin or -Pablo polypeptide. A polypeptide of the invention can also encode biologically functional polypeptides or peptides which have variant amino acid sequences, such as with changes selected based on considerations such as the relative hydropathic score of the amino acids being exchanged. These variant sequences are those isolated from natural sources or induced in the sequences disclosed herein using a mutagenic procedure such as site-directed mutagenesis.

Preferably, the expression vectors of the present invention comprise polynucleotides that encode polypeptides comprising the amino acid residue sequence of SEQ ID NO:, SEQ ID NO:4, SEQ ID NO:6, SEQ ID NO:8 or SEQ ID NO:10. An expression vector can include a pancortin or Pablo polypeptide coding region itself of any of the pancortin or Pablo polypeptides noted above or it can contain coding regions bearing selected alterations or modifications in the basic coding region of such a pancortin or Pablo polypeptide. Alternatively, such vectors or fragments can code larger polypeptides or polypeptides which nevertheless include the basic coding region. In any event, it should be appreciated that due to codon redundancy as well as biological functional equivalence, this aspect of the invention is not limited to the particular DNA molecules corresponding to the polypeptide sequences noted above.

Exemplary vectors include the mammalian expression vectors of the pCMV family including pCMV6b and pCMV6c (Chiron Corp., Emeryville Calif.). In certain cases, and specifically in the case of these individual mammalian expression vectors, the resulting constructs can require co-transfection with a vector containing a selectable marker such as pSV2neo. Via co-transfection into a dihydrofolate reductase-deficient Chinese hamster ovary cell line, such as DG44, clones expressing pancortin or Pablo polypeptides by virtue of DNA incorporated into such expression vectors can be detected.

A DNA molecule, gene or polynucleotide of the present invention can be incorporated into a vector by a number of techniques which are well known in the art. For instance, the vector pUC18 has been demonstrated to be of particular value Likewise, the related vectors M13 mp18 and M13 mp19 can be used in certain embodiments of the invention, in particular, in performing dideoxy sequencing.

An expression vector of the present invention is useful both as a means for preparing quantities of the pancortin or Pablo polypeptide-encoding DNA itself, and as a means for preparing the encoded polypeptide and peptides. It is contemplated that where pancortin or Pablo polypeptides of the invention are made by recombinant means, one can employ either prokaryotic or eukaryotic expression vectors as shuttle systems. However, in that prokaryotic systems are usually incapable of correctly processing precursor polypeptides and, in particular, such systems are incapable of correctly processing membrane associated eukaryotic polypeptides, and since eukaryotic pancortin or Pablo polypeptides are anticipated using the teaching of the disclosed invention, one likely expresses such sequences in eukaryotic hosts. However, even where the DNA segment encodes a eukaryotic pancortin or Pablo polypeptide, it is contemplated that prokaryotic expression can have some additional applicability. Therefore, the invention can be used in combination with vectors which can shuttle between the eukaryotic and prokaryotic cells. Such a system is described herein which allows the use of bacterial host cells as well as eukaryotic host cells.

Where expression of recombinant pancortin or Pablo polypeptides is desired and a eukaryotic host is contemplated, it is most desirable to employ a vector such as a plasmid, that incorporates a eukaryotic origin of replication. Additionally, for the purposes of expression in eukaryotic systems, one desires to position the pancortin or Pablo encoding sequence adjacent to, and under the control of, an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of or downstream with respect to the promoter chosen. Furthermore, where eukaryotic expression is anticipated, one would typically desire to incorporate into the transcriptional unit, which includes the pancortin or Pablo polypeptide, an appropriate polyadenylation site.

The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these 4 plasmids in containing a translation enhancer in the sequence prior to the polylinker. While they are not directly derived from the pCMV1-5 series of vectors, the functionally similar pCMV6b and c vectors are available from the Chiron Corp. (Emeryville, Calif.) and are identical except for the orientation of the polylinker region which is reversed in one relative to the other.

The universal components of the pCMV plasmids are as follows. The vector backbone is pTZ18R (Pharmacia), and contains a bacteriophage f1 origin of replication for production of single stranded DNA and an ampicillin-resistance gene. The CMV region consists of nucleotides −760 to +3 of the powerful promoter-regulatory region of the human cytomegalovirus (Towne stain) major immediate early gene (Thomsen et al., 1984; Boshart et al., 1985). The human growth hormone fragment (hGH) contains transcription termination and poly-adenylation signals representing sequences 1533 to 2157 of this gene (Seeburg, 1982). There is an Alu middle repetitive DNA sequence in this fragment. Finally, the SV40 origin of replication and early region promoter-enhancer derived from the pcD-X plasmid (HindII to PstI fragment) described in (Okayama et al., 1983). The promoter in this fragment is oriented such that transcription proceeds away from the CMV/hGH expression cassette.

The pCMV plasmids are distinguishable from each other by differences in the polylinker region and by the presence or absence of the translation enhancer. The starting pCMV1 plasmid has been progressively modified to render an increasing number of unique restriction sites in the polylinker region. To create pCMV2, one of two EcoRI sites in pCMV1 were destroyed. To create pCMV3, pCMV1 was modified by deleting a short segment from the SV40 region (StuI to EcoRI), and in so doing made unique the PstI, SalI, and BamHI sites in the polylinker. To create pCMV4, a synthetic fragment of DNA corresponding to the 5′-untranslated region of a mRNA transcribed from the CMV promoter was added. The sequence acts as a translational enhancer by decreasing the requirements for initiation factors in polypeptide synthesis (Jobling et al., 1987; Browning et al., 1988). To create pCMV5, a segment of DNA (HpaI to EcoRI) was deleted from the SV40 origin region of pCMV1 to render unique all sites in the starting polylinker.

The pCMV vectors have been successfully expressed in simian COS cells, mouse L cells, CHO cells, and HeLa cells. In several side by side comparisons they have yielded 5- to 10-fold higher expression levels in COS cells than SV40-based vectors. The pCMV vectors have been used to express the LDL receptor, nuclear factor 1, GS alpha polypeptide, polypeptide phosphatase, synaptophysin, synapsin, insulin receptor, influenza hemmagglutinin, androgen receptor, sterol 26-hydroxylase, steroid 17- and 21-hydroxylase, cytochrome P-450 oxidoreductase, beta-adrenergic receptor, folate receptor, cholesterol side chain cleavage enzyme, and a host of other cDNAs. It should be noted that the SV40 promoter in these plasmids can be used to express other genes such as dominant selectable markers. Finally, there is an ATG sequence in the polylinker between the HindIII and PstI sites in pCMU that can cause spurious translation initiation. This codon should be avoided if possible in expression plasmids. A paper describing the construction and use of the parenteral pCMV1 and pCMV4 vectors has been published (Anderson et al., 1989b).

In yet another embodiment, the present invention provides recombinant host cells transformed, infected or transfected with polynucleotides that encode pancortin or Pablo polypeptides, as well as transgenic cells derived from those transformed or transfected cells: Preferably, the recombinant host cells of the present invention are transfected with a polynucleotide of SEQ ID NO:1, SEQ ID NO:3, SEQ ID NO: 5, SEQ ID NO:7 or SEQ ID NO 9. Means of transforming or transfecting cells with exogenous polynucleotide such as DNA molecules are well known in the art and include techniques such as calcium phosphate-mediated or DEAE-dextran-mediated transfection, protoplast fusion, electroporation, liposome mediated transfection, direct microinjection and adenovirus infection (Sambrook, Fritsch and Maniatis, 1989).

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

The use of adenovirus as a vector for cell transfection is well known in the art. Adenovirus vector-mediated cell transfection has been reported for various cells (Stratford-Perricaudet, et al. 1992).

A transfected cell can be prokaryotic or eukaryotic. Preferably, the host cells of the invention are eukaryotic host cells. The recombinant host cells of the invention may be COS-1 cells. Where it is of interest to produce a human polypeptide, cultured mammalian or human cells are of particular interest.

In another aspect, the recombinant host cells of the present invention are prokaryotic host cells. Preferably, the recombinant host cells of the invention are bacterial cells of the DH5 α strain of Escherichia coli. In general, prokaryotes are preferred for the initial cloning of DNA sequences and constructing the vectors useful in the invention. For example, E. coli K12 strains can be particularly useful. Other microbial strains which can be used include E. coli B, and E. coliₓ 1976 (ATCC No. 31537). These examples are, of course, intended to be illustrative rather than limiting.

Prokaryotes can also be used for expression. The aforementioned strains, as well as E. coli W3110 (ATCC No. 273325), bacilli such as Bacillus subtilis, or other enterobacteriaceae such as Salmonella typhimurium or Serratia marcesans, and various Pseudomonas species can be used.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli can be transformed using pBR322, a plasmid derived from an E. coli species (Bolivar, et al. 1977). pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the β-lactamase (penicillinase) and lactose promoter systems (Chang, et al. 1978; Itakura., et al. 1977, Goeddel, et al. 1979; Goeddel, et al. 1980) and a tryptophan (TRP) promoter system (Siebwenlist et al. 1980). While these are the most commonly used, other microbial promoters have been discovered and utilized, and details concerning their nucleotide sequences have been published, enabling a skilled worker to introduce functional promoters into plasmid vectors (Siebwenlist, et al. 1980).

In addition to prokaryotes, eukaryotic microbes such as yeast can also be used. Saccharomyces cerevisiase or common baker's yeast is the most commonly used among eukaryotic microorganisms, although a number of other strains are commonly available. For expression in Saccharomyces, the plasmid YRp7, for example, is commonly used (Stinchcomb, et al. 1979; Kingsman, et al. 1979; Tschemper, et al. 1980). This plasmid already contains the trpl gene which provides a selection marker for a mutant strain of yeast lacking the ability to grow in tryptophan, for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoter sequences in yeast vectors include the promoters for 3-phosphoglycerate kinase (Hitzeman., et al. 1980) or other glycolytic enzymes (Hess, et al. 1968; Holland, et al. 1978) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also introduced into the expression vector downstream from the sequences to be expressed to provide polyadenylation of the mRNA and termination. Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin or replication and termination sequences is suitable.

In addition to microorganisms, cultures of cells derived from multicellular organisms can also be used as hosts. In principle, any such cell culture is workable, whether from vertebrate or invertebrate culture. However, interest has been greatest in vertebrate cells, and propagation of vertebrate cells in culture (tissue culture) has become a routine procedure in recent years. Examples of such useful host cell lines are AtT-20, VERO and HeLa cells, Chinese hamster ovary (CHO) cell lines, and W138, BHK, COSM6, COS-7, 293 and MDCK cell lines. Expression vectors for such cells ordinarily include (if necessary) an origin of replication, a promoter located upstream of the gene to be expressed, along with any necessary ribosome binding sites, RNA splice sites, polyadenylation site, and transcriptional terminator sequences.

For use in mammalian cells, the control functions on the expression vectors are often derived from viral material. For example, commonly used promoters are derived from polyoma, Adenovirus 2, Cytomegalovirus, Rous Sarcoma Virus (RSV) and most frequently Simian Virus 40 (SV40). The early and late promoters of SV40 virus are particularly useful because both are obtained easily from the virus as a fragment which also contains the SV40 viral origin of replication (Fiers, et al. 1978). Smaller or larger SV40 fragments can also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication can be provided by construction of the vector to include an exogenous origin, such as can be derived from SV40 or other viral (e.g., Polyoma, Adeno, VSV, BPV, CMV) source, or can be provided by the host cell chromosomal replication mechanism. If the vector is integrated into the host cell chromosome, the latter is often sufficient.

In yet another embodiment, the present invention contemplates a process or method of preparing polypeptides comprising transfecting cells with polynucleotide that encode pancortin or Pablo polypeptides to produce transformed host cells; and maintaining the transformed host cells under biological conditions sufficient for expression of the polypeptide. Preferably, the transformed host cells are eukaryotic cells. Alternatively, the host cells are prokaryotic cells. More preferably, the prokaryotic cells are bacterial cells of the DH5-α strain of Escherichia coli. Even more preferably, the polynucleotide transfected into the transformed cells comprise the nucleic acid sequence of SEQ ID NO:1, SEQ ID NO:3, SEQ ID NO:5, SEQ ID NO:7 or SEQ ID NO:9. Additionally, transfection is accomplished using an expression vector disclosed above.

A host cell used in the process is capable of expressing a functional, recombinant pancortin or Pablo polypeptide. A preferred host cell is a Chinese hamster ovary cell. However, a variety of cells are amenable to a process of the invention, for instance, yeast cells, human cell lines, and other eukaryotic cell lines known well to those of skill in the art.

Following transfection, the cell is maintained under culture conditions for a period of time sufficient for expression of a pancortin or Pablo polypeptide. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C., more preferably from about 30° C. to about 40° C. and, even more preferably about 37° C.

pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Other biological conditions needed for transfection and expression of an encoded polypeptide are well known in the art.

Transfected cells are maintained for a period of time sufficient for expression of a pancortin or Pablo polypeptide. A suitable time depends inter alia upon the cell type used and is readily determinable by a skilled artisan. Typically, maintenance time is from about 2 to about 14 days.

Recombinant pancortin or Pablo polypeptide is recovered or collected either from the transfected cells or the medium in which those cells are cultured. Recovery comprises isolating and purifying the pancortin or Pablo polypeptide. Isolation and purification techniques for polypeptides are well known in the art and include such procedures as precipitation, filtration, chromatography, electrophoresis and the like.

E. Transgenic Animals

In certain preferred embodiments, the invention pertains to nonhuman animals with somatic and germ cells having a functional disruption of at least one, and more preferably both, alleles of an endogenous pancortin or Pablo gene of the present invention. Accordingly, the invention provides viable animals having a mutated pancortin or Pablo gene, and thus lacking pancortin or Pablo activity. These animals will produce substantially reduced amounts of a pancortin or Pablo in response to stimuli that produce normal amounts of a pancortin or Pablo in wild type control animals. The animals of the invention are useful, for example, as standard controls by which to evaluate pancortin or Pablo inhibitors, as recipients of a normal human pancortin or Pablo gene to thereby create a model system for screening human pancortin or Pablo inhibitors in vivo, and to identify disease states for treatment with pancortin or Pablo inhibitors. The animals are also useful as controls for studying the effect of ligands on the pancortin or Pablo.

In the transgenic nonhuman animal of the invention, the pancortin or Pablo gene preferably is disrupted by homologous recombination between the endogenous allele and a mutant pancortin or Pablo polynucleotide, or portion thereof, that has been introduced into an embryonic stem cell precursor of the animal. The embryonic stem cell precursor is then allowed to develop, resulting in an animal having a functionally disrupted pancortin or Pablo gene. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. The animal may have one pancortin or Pablo gene allele functionally disrupted (i.e., the animal may be heterozygous for the mutation), or more preferably, the animal has both pancortin or Pablo gene alleles functionally disrupted (i.e., the animal can be homozygous for the mutation).

In one embodiment of the invention, functional disruption of both pancortin or Pablo gene alleles produces animals in which expression of the pancortin or Pablo gene product in cells of the animal is substantially absent relative to non-mutant animals. In another embodiment, the pancortin or Pablo gene alleles can be disrupted such that an altered (i.e., mutant) pancortin or Pablo gene product is produced in cells of the animal. A preferred nonhuman animal of the invention having a functionally disrupted pancortin or Pablo gene is a mouse. Given the essentially complete inactivation of pancortin or Pablo function in the homozygous animals of the invention and about 50% inhibition of pancortin or Pablo function in the heterozygous animals of the invention, these animals are useful as positive controls against which to evaluate the effectiveness of pancortin or Pablo inhibitors. For example, a stimulus that normally induces production or activity of pancortin or Pablo can be administered to a wild type animal (i.e., an animal having a non-mutant pancortin or Pablo gene) in the presence of a pancortin or Pablo inhibitor to be tested and production or activity of pancortin or Pablo by the animal can be measured. The pancortin or Pablo response in the wild type animal can then be compared to the pancortin or Pablo response in the heterozygous and homozygous animals of the invention, to determine the percent of maximal pancortin or Pablo inhibition of the test inhibitor.

Additionally, the animals of the invention are useful for determining whether a particular disease condition involves the action of pancortin or Pablo and thus can be treated by a pancortin or Pablo inhibitor. For example, an attempt can be made to induce a disease condition in an animal of the invention having a functionally disrupted pancortin or Pablo gene. Subsequently, the susceptibility or resistance of the animal to the disease condition can be determined. A disease condition that is treatable with a pancortin or Pablo inhibitor can be identified based upon resistance of an animal of the invention to the disease condition. Another aspect of the invention pertains to a transgenic nonhuman animal having a functionally disrupted endogenous pancortin or Pablo gene but which also carries in its genome, and expresses, a transgene encoding a heterologous pancortin or Pablo (i.e., a pancortin or Pablo from another species). Preferably, the animal is a mouse and the heterologous pancortin or Pablo is a human pancortin or Pablo. An animal of the invention which has been reconstituted with human pancortin or Pablo can be used to identify agents that inhibit human pancortin or Pablo in vivo. For example, a stimulus that induces production and/or activity of pancortin or Pablo can be administered to the animal in the presence and absence of an agent to be tested and the pancortin or Pablo response in the animal can be measured. An agent that inhibits human pancortin or Pablo in vivo can be identified based upon a decreased pancortin or Pablo response in the presence of the agent compared to the pancortin or Pablo response in the absence of the agent. As used herein, a “transgene” is exogenous DNA which is integrated into the genome of a cell from which a transgenic animal develops and which remains in the genome of the mature animal, thereby directing the expression of an encoded gene product in one or more cell types or tissues of the transgenic animal.

Yet another aspect of the invention pertains to a polynucleotide construct for functionally disrupting a pancortin or Pablo gene in a host cell. The nucleic acid construct comprises: a) a nonhomologous replacement portion; b) a first homology region located upstream of the nonhomologous replacement portion, the first homology region having a nucleotide sequence with substantial identity to a first pancortin or Pablo gene sequence; and c) a second homology region located downstream of the nonhomologous replacement portion, the second homology region having a nucleotide sequence with substantial identity to a second pancortin or Pablo gene sequence, the second pancortin or Pablo gene sequence having a location downstream of the first pancortin or Pablo gene sequence in a naturally occurring endogenous pancortin or Pablo gene. Additionally, the first and second homology regions are of sufficient length for homologous recombination between the nucleic acid construct and an endogenous pancortin or Pablo gene in a host cell when the nucleic acid molecule is introduced into the host cell. As used herein, a “homologous recombinant animal” is a non-human animal, preferably a mammal, more preferably a mouse, in which an endogenous pancortin or Pablo gene has been altered by homologous recombination between the endogenous gene and an exogenous DNA molecule introduced into a cell of the animal, e.g., an embryonic cell of the animal, prior to development of the animal.

In a preferred embodiment, the nonhomologous replacement portion comprises a positive selection expression cassette, preferably including a neomycin phosphotransferase gene operatively linked to a regulatory element(s). In another preferred embodiment, the nucleic acid construct also includes a negative selection expression cassette distal to either the upstream or downstream homology regions. A preferred negative selection cassette includes a herpes simplex virus thymidine kinase gene operatively linked to a regulatory element(s). Another aspect of the invention pertains to recombinant vectors into which the nucleic acid construct of the invention has been incorporated.

Yet another aspect of the invention pertains to host cells into which the nucleic acid construct of the invention has been introduced to thereby allow homologous recombination between the nucleic acid construct and an endogenous pancortin or Pablo gene of the host cell, resulting in functional disruption of the endogenous pancortin or Pablo gene. The host cell can be a mammalian cell that normally expresses pancortin or Pablo, such as a human neuron, or a pluripotent cell, such as a mouse embryonic stem cell. Further development of an embryonic stem cell into which the nucleic acid construct has been introduced and homologously recombined with the endogenous pancortin or Pablo gene produces a transgenic nonhuman animal having cells that are descendant from the embryonic stem cell and thus carry the pancortin or Pablo gene disruption in their genome. Animals that carry the pancortin or Pablo gene disruption in their germline can then be selected and bred to produce animals having the pancortin or Pablo gene disruption in all somatic and germ cells. Such mice can then be bred to homozygosity for the pancortin or Pablo gene disruption.

It is contemplated that in some instances the genome of a transgenic animal of the present invention will have been altered through the stable introduction of one or more of the pancortin or Pablo polynucleotide compositions described herein, either native, synthetically modified or mutated. As described herein, a “transgenic animal” refers to any animal, preferably a non-human mammal (e.g. mouse, rat, rabbit, squirrel, hamster, rabbits, guinea pigs, pigs, micro-pigs, prairie, baboons, squirrel monkeys and chimpanzees, etc), bird or an amphibian, in which one or more cells contain heterologous nucleic acid introduced by way of human intervention, such as by transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

The host cells of the invention can also be used to produce non-human transgenic animals. The non-human transgenic animals can be used in screening assays designed to identify agents or compounds, e.g., drugs, pharmaceuticals, etc., which are capable of ameliorating detrimental symptoms of selected disorders such as nervous system disorders, e.g., psychiatric disorders or disorders affecting circadian rhythms and the sleep-wake cycle. For example, in one embodiment, a host cell of the invention is a fertilized oocyte or an embryonic stem cell into which pancortin or Pablo polypeptide-coding sequences have been introduced. Such host cells can then be used to create non-human transgenic animals in which exogenous pancortin or Pablo gene sequences have been introduced into their genome or homologous recombinant animals in which endogenous pancortin or Pablo gene sequences have been altered. Such animals are useful for studying the function and/or activity of a pancortin or Pablo polypeptide and for identifying and/or evaluating modulators of pancortin or Pablo polypeptide activity.

A transgenic animal of the invention can be created by introducing pancortin or Pablo polypeptide encoding nucleic acid into the male pronuclei of a fertilized oocyte, e.g., by microinjection, retroviral infection, and allowing the oocyte to develop in a pseudopregnant female foster animal. The human pancortin or Pablo cDNA sequence of SEQ ID NO:1, SEQ ID NO:3, SEQ ID NO:5, SEQ ID NO:7 or SEQ ID NO:9. respectively, can be introduced as a transgene into the genome of a non-human animal.

Moreover, a non-human homologue of the human pancortin or Pablo gene, such as a mouse pancortin or Pablo gene, can be isolated based on hybridization to the human pancortin or Pablo cDNA (described above) and used as a transgene. Intronic sequences and polyadenylation signals can also be included in the transgene to increase the efficiency of expression of the transgene. A tissue-specific regulatory sequence(s) can be operably linked to the pancortin or Pablo transgene to direct expression of a pancortin or Pablo polypeptide to particular cells. Methods for generating transgenic animals via embryo manipulation and microinjection, particularly animals such as mice, have become conventional in the art and are described, for example, in U.S. Pat. No. 4,736,866, U.S. Pat. No. 4,870,009, U.S. Pat. No. 4,873,191 and in Hogan, 1986. Similar methods are used for production of other transgenic animals. A transgenic founder animal can be identified based upon the presence of the pancortin or Pablo transgene in its genome and/or expression of pancortin or Pablo mRNA in tissues or cells of the animals. A transgenic founder animal can then be used to breed additional animals carrying the transgene. Moreover, transgenic animals carrying a transgene encoding a pancortin or Pablo polypeptide can further be bred to other transgenic animals carrying other transgenes.

To create a homologous recombinant animal, a vector is prepared which contains at least a fragment of a pancortin or Pablo gene into which a deletion, addition or substitution has been introduced to thereby alter, e.g., functionally disrupt, the pancortin or Pablo gene. The pancortin or Pablo gene can be a human gene (e.g., from a human genomic clone isolated from a human genomic library screened with the cDNA of SEQ ID NO:1, SEQ ID NO:3, SEQ ID NO:5, SEQ ID NO:7 or SEQ ID NO:9), but more preferably is a non-human homologue of a human pancortin or Pablo gene. For example, a mouse pancortin or Pablo gene can be isolated from a mouse genomic DNA library using the pancortin or Pablo cDNA of SEQ ID NO:1, SEQ ID NO:3, SEQ ID NO:5, SEQ ID NO:7 or SEQ ID NO:9 respectively, as a probe. The mouse pancortin or Pablo gene then can be used to construct a homologous recombination vector suitable for altering an endogenous pancortin or Pablo gene in the mouse genome. In a preferred embodiment, the vector is designed such that, upon homologous recombination, the endogenous pancortin or Pablo gene is functionally disrupted (i.e., no longer encodes a functional protein; also referred to as a “knock out” vector.

Alternatively, the vector can be designed such that, upon homologous recombination, the endogenous pancortin or Pablo gene is mutated or otherwise altered but still encodes functional protein (e.g., the upstream regulatory region can be altered to thereby alter the expression of the endogenous pancortin or Pablo polypeptide). In the homologous recombination vector, the altered fragment of the pancortin or Pablo gene is flanked at its 5′ and 3′ ends by additional nucleic acid of the pancortin or Pablo to allow for homologous recombination to occur between the exogenous pancortin or Pablo gene carried by the vector and an endogenous pancortin or Pablo gene in an embryonic stem cell. The additional flanking pancortin or Pablo nucleic acid is of sufficient length for successful homologous recombination with the endogenous gene.

Typically, several kilobases of flanking DNA (both at the 5′ and 3′ ends) are included in the vector (see e.g., Thomas and Capecchi, 1987, for a description of homologous recombination vectors). The vector is introduced into an embryonic stem cell line (e.g., by electroporation) and cells in which the introduced pancortin or Pablo gene has homologously recombined with the endogenous pancortin or Pablo gene are selected (see e.g., Li et al., 1992). The selected cells are then injected into a blastocyst of an animal (e.g., a mouse) to form aggregation chimeras (see e.g., Bradley, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, 1991; and in PCT International Publication Nos. WO 90/11354; WO 91/01140; and WO 93/04169.

In another embodiment, transgenic non-human animals can be produced which contain selected systems which allow for regulated expression of the transgene. One example of such a system is the cre/loxP recombinase system of bacteriophage PL. For a description of the cre/loxP recombinase system, see, e.g., Lakso et al., 1992. Another example of a recombinase system is the FLP recombinase system of Saccharomyces cerevisiae (O'Gonnan et al., 1991). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al., 1997, and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then is transferred to a pseudopregnant female foster animal. The offspring borne of this female foster animal will be a clone of the animal from which the cell, e.g., the somatic cell, is isolated.

F. Uses and Methods of the Invention

The nucleic acid molecules, polypeptides, polypeptide homologues, modulators, antibodies, vectors and host cells described herein can be used in one or more of the following methods: a) drug screening assays; b) diagnostic assays particularly in disease identification, allelic screening and pharmocogenetic testing; c) methods of treatment; d) pharmacogenomics; and e) monitoring of effects during clinical trials. A polypeptide of the invention can be used as a drug target for developing agents to modulate the activity of a pancortin-Pablo polypeptide dimer. The isolated nucleic acid molecules of the invention can be used to express pancortin and Pablo polypeptide (e.g., via a recombinant expression vector in a host cell or in gene therapy applications), to detect pancortin and Pablo mRNA (e.g., in a biological sample) or a naturally occurring or recombinantly generated genetic mutation in a pancortin and Pablo gene, and to modulate pancortin and Pablo polypeptide activity, as described further below. In addition, the pancortin and Pablo polypeptides can be used to screen drugs or compounds which modulate polypeptide activity. Moreover, the anti-pancortin and anti-Pablo antibodies of the invention can be used to detect and isolate a pancortin or Pablo polypeptide, particularly fragments of a pancortin and Pablo polypeptides present in a biological sample, and to modulate pancortin and Pablo polypeptide activity.

Drug Screening Assays

The invention provides methods for identifying compounds or agents that can be used to treat disorders characterized by (or associated with) aberrant or abnormal pancortin and Pablo acid expression and/or abnormal pancortin-Pablo polypeptide activity. These methods are also referred to herein as drug screening assays and typically include the step of screening a candidate/test compound or agent to identify compounds that are an agonist or antagonist of a pancortin or Pablo polypeptide, and specifically for the ability to interact with (e.g., bind to) a pancortin or Pablo polypeptide, to modulate the interaction of a pancortin or Pablo polypeptide and a target molecule, and/or to modulate pancortin or Pablo nucleic acid expression and/or pancortin or Pablo polypeptide activity. Candidate/test compounds or agents which have one or more of these abilities can be used as drugs to treat disorders characterized by aberrant or abnormal pancortin or Pablo nucleic acid expression and/or pancortin or Pablo polypeptide activity. Candidate/test compounds include, for example, 1) peptides such as soluble peptides, including Ig-tailed fusion peptides and members of random peptide libraries and combinatorial chemistry-derived molecular libraries made of D- and/or L-configuration amino acids; 2) phosphopeptides (e.g., members of random and partially degenerate, directed phosphopeptide libraries, see, e.g., Songyang et al., 1993); 3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); and 4) small organic and inorganic molecules (e.g., molecules obtained from combinatorial and natural product libraries). In one embodiment, the invention provides assays for screening candidate/test compounds which interact with (e.g., bind to) a pancortin or Pablo polypeptide. Typically, the assays are recombinant cell based or cell-free assays which include the steps of combining a cell expressing a pancortin polypeptide or a pancortin-Pablo polypeptide or a bioactive fragment thereof, or an isolated pancortin polypeptide or a pancortin-Pablo polypeptide, and a candidate/test compound, e.g., under conditions which allow for interaction of (e.g., binding of) the candidate/test compound to the pancortin polypeptide or the pancortin-Pablo polypeptide or fragment thereof to form a complex, and detecting the formation of a complex, in which the ability of the candidate compound to interact with (e.g., bind to) the pancortin polypeptide or pancortin-Pablo polypeptide or fragment thereof is indicated by the presence of the candidate compound in the complex. Formation of complexes between the pancortin polypeptide or a pancortin-Pablo polypeptide and the candidate compound can be detected using competition binding assays, and can be quantitated, for example, using standard immunoassays.

In another embodiment, the invention provides screening assays to identify candidate/test compounds which modulate (e.g., stimulate or inhibit) the interaction (and most likely polypeptide activity as well) between a pancortin polypeptide or a pancortin-Pablo polypeptide and a molecule (target molecule) with which the pancortin polypeptide or pancortin-Pablo polypeptide normally interacts. Examples of such target molecules include proteins in the same signaling path as the pancortin polypeptide or pancortin-Pablo polypeptide, e.g., proteins which may function upstream (including both stimulators and inhibitors of activity) or downstream of the pancortin polypeptide or pancortin-Pablo polypeptide in, for example, an apoptotic signaling pathway or in a pathway involving a pancortin polypeptide or pancortin-Pablo polypeptide activity, e.g., a Bcl-X_{L}-Pablo-pancortin interaction. Typically, the assays are recombinant cell based assays which include the steps of combining a cell expressing a pancortin polypeptide or pancortin-Pablo polypeptide, or a bioactive fragment thereof, a pancortin polypeptide or pancortin-Pablo polypeptide target molecule (e.g., a pancortin polypeptide or pancortin-Pablo ligand) and a candidate/test compound, e.g., under conditions wherein but for the presence of the candidate compound, the pancortin polypeptide or pancortin-Pablo polypeptide or biologically active fragment thereof interacts with (e.g., binds to) the target molecule, and detecting the formation of a complex which includes the pancortin polypeptide or pancortin-Pablo polypeptide and the target molecule or detecting the interaction/reaction of the pancortin polypeptide or pancortin-Pablo polypeptide and the target molecule.

Detection of complex formation can include direct quantitation of the complex by, for example, measuring inductive effects of the pancortin polypeptide or pancortin-Pablo polypeptide. A statistically significant change, such as a decrease, in the interaction of the pancortin polypeptide or pancortin-Pablo polypeptide and target molecule (e.g., in the formation of a complex between the pancortin polypeptide or pancortin-Pablo polypeptide and the target molecule) in the presence of a candidate compound (relative to what is detected in the absence of the candidate compound) is indicative of a modulation (e.g., stimulation or inhibition) of the interaction between the pancortin polypeptide or pancortin-Pablo polypeptide and the target molecule. Modulation of the formation of complexes between the pancortin polypeptide or pancortin-Pablo polypeptide and the target molecule can be quantitated using, for example, an immunoassay.

To perform cell free drug screening assays, it is desirable to immobilize either the pancortin polypeptide or pancortin-Pablo polypeptide or its target molecule to facilitate separation of complexes from uncomplexed forms of one or both of the proteins, as well as to accommodate automation of the assay. Interaction (e.g., binding) of the pancortin polypeptide or pancortin-Pablo polypeptide to a target molecule, in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows the protein to be bound to a matrix. For example, glutathione-S-transferase/pancortin polypeptide or pancortin-Pablo fusion proteins can be adsorbed onto glutathione sepharose beads (Sigma Chemical, St. Louis, Mo.) or glutathione derivatized microtitre plates, which are then combined with the cell lysates (e.g., ³⁵S labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of pancortin polypeptide or pancortin-Pablo-binding protein found in the bead fraction quantitated from the gel using standard electrophoretic techniques.

Other techniques for immobilizing proteins on matrices can also be used in the drug screening assays of the invention. For example, either the pancortin polypeptide or pancortin-Pablo polypeptide dimer or its target molecule can be immobilized utilizing conjugation of biotin and streptavidin. Biotinylated pancortin polypeptide or pancortin-Pablo polypeptide molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with a pancortin polypeptide or pancortin-Pablo polypeptide but which do not interfere with binding of the protein to its target molecule can be derivatized to the wells of the plate, and pancortin polypeptide or pancortin-Pablo polypeptide trapped in the wells by antibody conjugation. As described above, preparations of a pancortin polypeptide or pancortin-Pablo polypeptide-binding protein and a candidate compound are incubated in the pancortin polypeptide or pancortin-Pablo polypeptide-presenting wells of the plate, and the amount of complex trapped in the well can be quantitated. Methods for detecting such complexes, in addition to those described above for the GST-immobilized complexes, include immunodetection of complexes using antibodies reactive with the pancortin polypeptide or pancortin-Pablo polypeptide target molecule, or which are reactive with pancortin polypeptide or pancortin-Pablo polypeptide and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant or abnormal pancortin polypeptide or pancortin-Pablo polypeptide activity. This method typically includes the step of assaying the ability of the compound or agent to modulate the expression of the pancortin or pancortin-Pablo nucleic acid or the activity of the pancortin polypeptide or pancortin-Pablo polypeptide thereby identifying a compound for treating a disorder characterized by aberrant or abnormal pancortin or pancortin-Pablo nucleic acid expression or pancortin polypeptide or pancortin-Pablo polypeptide activity. Methods for assaying the ability of the compound or agent to modulate the expression of the pancortin or pancortin-Pablo nucleic acid or activity of the pancortin polypeptide or pancortin-Pablo polypeptide are typically cell-based assays. For example, cells which are sensitive to ligands which transduce signals via a pathway involving a pancortin polypeptide or pancortin-Pablo polypeptide can be induced to overexpress a pancortin polypeptide or pancortin-Pablo polypeptide in the presence and absence of a candidate compound.

Candidate compounds which produce a statistically significant change in pancortin polypeptide or pancortin-Pablo polypeptide-dependent responses (either stimulation or inhibition) can be identified. In one embodiment, expression of pancortin or pancortin-Pablo nucleic acid or activity of a pancortin polypeptide or pancortin-Pablo polypeptide is modulated in cells and the effects of candidate compounds on the readout of interest (such as apoptosis) are measured. For example, the expression of genes which are up- or down-regulated in response to a pancortin polypeptide or pancortin-Pablo polypeptide-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of a pancortin polypeptide or pancortin-Pablo polypeptide or pancortin polypeptide or pancortin-Pablo polypeptide target molecules can also be measured, for example, by immunoblotting.

Alternatively, modulators of pancortin or pancortin-Pablo expression (e.g., compounds which can be used to treat a disorder characterized by aberrant or abnormal pancortin or pancortin-Pablo nucleic acid expression or pancortin or pancortin-Pablo polypeptide activity) can be identified in a method wherein a cell is contacted with a candidate compound and the expression of pancortin or pancortin-Pablo mRNA or protein in the cell is determined. The level of expression of pancortin or pancortin-Pablo mRNA or protein in the presence of the candidate compound is compared to the level of expression of pancortin or pancortin-Pablo mRNA or protein in the absence of the candidate compound. The candidate compound can then be identified as a modulator of pancortin or pancortin-Pablo nucleic acid expression based on this comparison and be used to treat a disorder characterized by aberrant pancortin or, pancortin-Pablo nucleic acid expression. For example, when expression of pancortin or pancortin-Pablo mRNA or protein is greater (statistically significantly greater) in the presence of the candidate compound than in its absence, the candidate compound is identified as a stimulator of pancortin or pancortin-Pablo nucleic acid expression. Alternatively, when pancortin or pancortin-Pablo nucleic acid expression is less (statistically significantly less) in the presence of the candidate compound than in its absence, the candidate compound is identified as an inhibitor of pancortin or pancortin-Pablo nucleic acid expression. The level of pancortin or pancortin-Pablo nucleic acid expression in the cells can be determined by methods described herein for detecting pancortin or pancortin-Pablo mRNA or protein.

In certain aspects of the invention, pancortin or pancortin-Pablo polypeptides or portions thereof can be used as “bait proteins” in a two-hybrid assay or three-hybrid assay (see, e.g., U.S. Pat. No. 5,283,317; U.S. Statutory Invention Registration No. H1,892; Zervos et al., 1993; Madura et al., 1993; Bartel et al., 1993(b); Iwabuchi et al., 1993; International Application No. WO 94/10300), to identify other proteins, which bind to or interact with pancortin or pancortin-Pablo and are involved in pancortin or pancortin-Pablo activity. Such pancortin or pancortin-Pablo-binding proteins are also likely to be involved in the propagation of signals by the pancortin or pancortin-Pablo polypeptides or pancortin or pancortin-Pablo targets as, for example, downstream elements of a apoptosis-mediated signaling pathway. Alternatively, such pancortin or pancortin-Pablo-binding proteins may be pancortin or pancortin-Pablo inhibitors.

Thus, in certain embodiments, the invention contemplates determining protein:protein interactions. The yeast two-hybrid system is extremely useful for studying protein:protein interactions. Variations of the system are available for screening yeast phagemid (Harper et al., 1993; Elledge et al., 1991) or plasmid (Bartel et al., 1993(b), Bartel 1993(a); Finley and Brent, 1994) cDNA libraries to clone interacting proteins, as well as for studying known protein pairs. Recently, a two-hybrid method for high volume screening for specific inhibitors of protein:protein interactions and a two-hybrid screen that identifies many different interactions between protein pairs at once have been described (see, U.S. Statutory Invention Registration No. H1,892).

The success of the two-hybrid system relies upon the fact that the DNA binding and polymerase activation domains of many transcription factors, such as GAL4, can be separated and then rejoined to restore functionality (Morin et al., 1993). Briefly, the assay utilizes two different DNA constructs. In one construct, the gene that codes for a pancortin polypeptide, a Pablo polypeptide, or both, is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a pancortin, or a Pablo, or a pancortin-Pablo dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the pancortin or pancortin-Pablo polypeptide.

Modulators of pancortin or pancortin-Pablo polypeptide activity and/or pancortin or pancortin-Pablo nucleic acid expression identified according to these drug screening assays can be used to treat, for example, nervous system disorders. These methods of treatment include the steps of administering the modulators of pancortin or pancortin-Pablo polypeptide activity and/or nucleic acid expression, e.g., in a pharmaceutical composition as described herein, to a subject in need of such treatment, e.g., a subject with a disorder described herein.

Diagnostic Assays

The invention further provides a method for detecting the presence of a pancortin or pancortin-Pablo polypeptide or pancortin or pancortin-Pablo nucleic acid molecule, or fragment thereof, in a biological sample. The method involves contacting the biological sample with a compound or an agent capable of detecting pancortin or pancortin-Pablo polypeptide or mRNA such that the presence of pancortin or pancortin-Pablo polypeptide/encoding nucleic acid molecule is detected in the biological sample. A preferred agent for detecting pancortin or pancortin-Pablo mRNA is a labeled or labelable nucleic acid probe capable of hybridizing to pancortin or pancortin-Pablo mRNA. The nucleic acid probe can be, for example, the full-length pancortin or pancortin-Pablo cDNA of SEQ ID NO: 1, SEQ ID NO: 3, SEQ ID NO:5, SEQ ID NO:7, SEQ ID NO:9, or a fragment thereof, such as an oligonucleotide of at least 15, 30, 50, 100, 250 or 500 nucleotides in length and sufficient to specifically hybridize under stringent conditions to pancortin or pancortin-Pablo mRNA. A preferred agent for detecting pancortin or pancortin-Pablo polypeptide is a labeled or labelable antibody capable of binding to pancortin or a dimer of pancortin-Pablo. Antibodies can be polyclonal, or more preferably, monoclonal. An intact antibody, or a fragment thereof (e.g., Fab or F(ab′)2) can be used. The term “labeled or labelable,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect pancortin or pancortin-Pablo mRNA or protein in a biological sample in vitro as well as in vivo. For example, in vitro techniques for detection of pancortin or pancortin-Pablo mRNA include Northern hybridizations and in situ hybridizations. In vitro techniques for detection of pancortin or pancortin-Pablo polypeptide include enzyme linked immunosorbent assays (ELISAs), Western blots, immunoprecipitations and immunofluorescence. Alternatively, pancortin or pancortin-Pablo polypeptide can be detected in vivo in a subject by introducing into the subject a labeled anti-pancortin or anti-pancortin-Pablo antibody. For example, the antibody can be labeled with a radioactive marker whose presence and location in a subject can be detected by standard imaging techniques. Particularly useful are methods which detect the allelic variant of a pancortin or pancortin-Pablo polypeptide expressed in a subject and methods which detect fragments of a pancortin or pancortin-Pablo polypeptide in a sample.

The invention also encompasses kits for detecting the presence of a pancortin or pancortin-Pablo polypeptide in a biological sample. For example, the kit can comprise reagents such as a labeled or labelable compound or agent capable of detecting pancortin or pancortin-Pablo polypeptide or mRNA in a biological sample; means for determining the amount of pancortin or pancortin-Pablo polypeptide in the sample; and means for comparing the amount of pancortin or pancortin-Pablo polypeptide in the sample with a standard. The compound or agent can be packaged in a suitable container. The kit can further comprise instructions for using the kit to detect pancortin or pancortin-Pablo mRNA or protein.

The methods of the invention can also be used to detect naturally occurring genetic mutations in a pancortin or pancortin-Pablo gene, thereby determining if a subject with the mutated gene is at risk for a disorder characterized by aberrant or abnormal pancortin or pancortin-Pablo nucleic acid expression or pancortin or pancortin-Pablo polypeptide activity as described herein. In preferred-embodiments, the methods include detecting, in a sample of cells from the subject, the presence or absence of a genetic mutation characterized by at least one of an alteration affecting the integrity of a gene encoding a pancortin or pancortin-Pablo polypeptide, or the misexpression of the pancortin or pancortin-Pablo gene. For example, such genetic mutations can be detected by ascertaining the existence of at least one of 1) a deletion of one or more nucleotides from a pancortin or pancortin-Pablo gene; 2) an addition of one or more nucleotides to a pancortin or pancortin-Pablo gene; 3) a substitution of one or more nucleotides of a pancortin or pancortin-Pablo gene, 4) a chromosomal rearrangement of a pancortin or pancortin-Pablo gene; 5) an alteration in the level of a messenger RNA transcript of a pancortin or pancortin-Pablo gene, 6) aberrant modification of a pancortin or pancortin-Pablo gene, such as of the methylation pattern of the genomic DNA, 7) the presence of a non-wild type splicing pattern of a messenger RNA transcript of a pancortin or pancortin-Pablo gene, 8) a non-wild type level of a pancortin or pancortin-Pablo-protein, 9) allelic loss of a pancortin or pancortin-Pablo gene, and 10) inappropriate post-translational modification of a pancortin or pancortin-Pablo-protein. As described herein, there are a large number of assay techniques known in the art that can be used for detecting mutations in a pancortin or pancortin-Pablo gene.

In certain embodiments, detection of the mutation involves the use of a probe/primer in a polymerase chain reaction (PCR) (see, e.g. U.S. Pat. No. 4,683,195 and U.S. Pat. No. 4,683,202), such as anchor PCR or RACE PCR, or, alternatively, in a ligation chain reaction (LCR), the latter of which can be particularly useful for detecting point mutations in the pancortin or pancortin-Pablo-gene (see Abravaya et al., 1995). This method can include the steps of collecting a sample of cells from a patient, isolating nucleic acid (e.g., genomic, mRNA or both) from the cells of the sample, contacting the nucleic acid sample with one or more primers which specifically hybridize to a pancortin or pancortin-Pablo gene under conditions such that hybridization and amplification of the pancortin or pancortin-Pablo-gene (if present) occurs, and detecting the presence or absence of an amplification product, or detecting the size of the amplification product and comparing the length to a control sample.

In an alternative embodiment, mutations in a pancortin or pancortin-Pablo gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see U.S. Pat. No. 5,498,531 hereby incorporated by reference in its entirety) can be used to score for the presence of specific mutations by development or loss of a ribozyme cleavage site.

In yet another embodiment, any of a variety of sequencing reactions known in the art can be used to directly sequence the pancortin or pancortin-Pablo gene and detect mutations by comparing the sequence of the sample pancortin or pancortin-Pablo gene with the corresponding wild-type (control) sequence. Examples of sequencing reactions include those based on techniques developed by Maxim and Gilbert (1977) or Sanger (1977). A variety of automated sequencing procedures can be utilized when performing the diagnostic assays, including sequencing by mass spectrometry (see, e.g., International Application No. WO 94/16101; Cohen et al., 1996; and Griffin et al. 1993).

Other methods for detecting mutations in the pancortin or pancortin-Pablo gene include methods in which protection from cleavage agents is used to detect mismatched bases in RNA/RNA or RNA/DNA duplexes (Myers et al., 1985 (b); Cotton et al., 1988; Saleeba et al., 1992), electrophoretic mobility of mutant and wild type nucleic acid is compared (Orita et al., 1989; Cotton, 1993; and Hayashi, 1992), and movement of mutant or wild-type fragments in polyacrylamide gels containing a gradient of denaturant is assayed using denaturing gradient gel electrophoresis (Myers et al., 1985(a)). Examples of other techniques for detecting point mutations include, selective oligonucleotide hybridization, selective amplification, and selective primer extension.

Methods of Treatment

Another aspect of the invention pertains to methods for treating a subject, e.g., a human, having a disease or disorder characterized by (or associated with) aberrant or abnormal pancortin or pancortin-Pablo nucleic acid expression and/or pancortin or pancortin-Pablo polypeptide activity. These methods include the step of administering a pancortin or pancortin-Pablo polypeptide/gene modulator (agonist or antagonist) to the subject such that treatment occurs. The language “aberrant or abnormal pancortin or pancortin-Pablo polypeptide expression” refers to expression of a non-wild-type pancortin or pancortin-Pablo polypeptide or a non-wild-type level of expression of a pancortin or pancortin-Pablo polypeptide. Aberrant or abnormal pancortin or pancortin-Pablo polypeptide activity refers to a non-wild-type pancortin or pancortin-Pablo polypeptide activity or a non-wild-type level of pancortin or pancortin-Pablo polypeptide activity. As the pancortin or pancortin-Pablo polypeptide is involved in a pathway involving signaling within cells, aberrant or abnormal pancortin or pancortin-Pablo polypeptide activity or expression interferes with the normal regulation of functions mediated by pancortin or pancortin-Pablo polypeptide signaling, and in particular brain cells. The terms “treating” or “treatment,” as used herein, refer to reduction or alleviation of at least one adverse effect or symptom of a disorder or disease, e.g., a disorder or disease characterized by or associated with abnormal or aberrant pancortin or pancortin-Pablo polypeptide activity or pancortin or pancortin-Pablo nucleic acid expression.

As used herein, a pancortin or pancortin-Pablo polypeptide/gene modulator is a molecule which can modulate pancortin or pancortin-Pablo nucleic acid expression and/or pancortin or pancortin-Pablo polypeptide activity. For example, a pancortin or pancortin-Pablo gene or protein modulator can modulate, e.g., upregulate (activate/agonize) or downregulate (suppress/antagonize), pancortin or pancortin-Pablo nucleic acid expression. In another example, a pancortin or pancortin-Pablo polypeptide/gene modulator can modulate (e.g., stimulate/agonize or inhibit/antagonize) pancortin or pancortin-Pablo polypeptide activity. If it is desirable to treat a disorder or disease characterized by (or associated with) aberrant or abnormal (non-wild-type) pancortin or pancortin-Pablo nucleic acid expression and/or pancortin or pancortin-Pablo polypeptide activity by inhibiting pancortin or pancortin-Pablo nucleic acid expression, a pancortin or pancortin-Pablo modulator can be an antisense molecule, e.g., a ribozyme, as described herein. Examples of antisense molecules which can be used to inhibit pancortin or pancortin-Pablo nucleic acid expression include antisense molecules which are complementary to a fragment of the 5′ untranslated region of SEQ ID NO: 1, SEQ ID NO: 3, SEQ ID NO:5, SEQ ID NO:7, SEQ ID NO:9, which also includes the start codon and antisense molecules which are complementary to a fragment of a 3′ untranslated region of SEQ ID NO: 1, SEQ ID NO: 3, SEQ ID NO:5, SEQ ID NO:7, SEQ ID NO:9.

A pancortin or pancortin-Pablo modulator that inhibits pancortin or pancortin-Pablo nucleic acid expression can also be a small molecule or other drug, e.g., a small molecule or drug identified using the screening assays described herein, which inhibits pancortin or pancortin-Pablo nucleic acid expression. If it is desirable to treat a disease or disorder characterized by (or associated with) aberrant or abnormal (non-wild-type) pancortin or pancortin-Pablo nucleic acid expression and/or pancortin or pancortin-Pablo polypeptide activity by stimulating pancortin or pancortin-Pablo nucleic acid expression, a pancortin or pancortin-Pablo modulator can be, for example, a nucleic acid molecule encoding a pancortin or pancortin-Pablo polypeptide (e.g., a nucleic acid molecule comprising a nucleotide sequence homologous to the nucleotide sequence of SEQ ID NO: 1, SEQ ID NO: 3, SEQ ID NO:5, SEQ ID NO:7, SEQ ID NO:9), or a small molecule or other drug, e.g., a small molecule (peptide) or drug identified using the screening assays described herein, which stimulates pancortin or pancortin-Pablo nucleic acid expression.

Alternatively, if it is desirable to treat a disease or disorder characterized by (or associated with) aberrant or abnormal (non-wild-type) pancortin or pancortin-Pablo nucleic acid expression and/or pancortin or pancortin-Pablo polypeptide activity by inhibiting pancortin or pancortin-Pablo polypeptide activity, a pancortin or pancortin-Pablo modulator can be an anti-pancortin or pancortin-Pablo antibody or a small molecule or other drug, e.g., a small molecule or drug identified using the screening assays described herein, which inhibits pancortin or pancortin-Pablo polypeptide activity. If it is desirable to treat a disease or disorder characterized by (or associated with) aberrant or abnormal (non-wild-type) pancortin or pancortin-Pablo nucleic acid expression and/or pancortin or pancortin-Pablo polypeptide activity by stimulating pancortin or pancortin-Pablo polypeptide activity, a pancortin or pancortin-Pablo modulator can be an active pancortin or pancortin-Pablo polypeptide or fragment thereof (e.g., a pancortin or pancortin-Pablo polypeptide or fragment thereof having an amino acid sequence which is homologous to the amino acid sequence of SEQ ID NO:2, SEQ ID NO: 4, SEQ ID NO:6, SEQ ID NO:8, SEQ ID NO:10, or a fragment thereof) or a small molecule or other drug, e.g., a small molecule or drug identified using the screening assays described herein, which stimulates pancortin or pancortin-Pablo polypeptide activity.

Other aspects of the invention pertain to methods for modulating a pancortin or pancortin-Pablo polypeptide mediated cell activity. These methods include contacting the cell with an agent (or a composition which includes an effective amount of an agent) which modulates pancortin or pancortin-Pablo polypeptide activity or pancortin or pancortin-Pablo nucleic acid expression such that a pancortin or pancortin-Pablo polypeptide mediated cell activity is altered relative to normal levels (for example, cAMP or phosphatidylinositol metabolism). As used herein, “a pancortin or pancortin-Pablo polypeptide mediated cell activity” refers to a normal or abnormal activity or function of a cell. Examples of pancortin or pancortin-Pablo polypeptide mediated cell activities include, but are not limited to: production or secretion of molecules, such as proteins, contraction, neuronal growth, cone guidance, axonal or dendritic regeneration or degeneration, proliferation, migration, differentiation, cell death, cell survival, reactive oxygen species, Ca²⁺, glutamate, phosphorylation of tyrosine, serine or threonine residues and caspase activation. In a preferred embodiment, the cell is a neural cell of the brain, e.g., a hippocampal cell. The term “altered” as used herein refers to a change, e.g., an increase or decrease, of a cell associated activity, particularly apoptosis.

In one embodiment, the agent stimulates pancortin or pancortin-Pablo polypeptide activity or pancortin or pancortin-Pablo nucleic acid expression. In another embodiment, the agent inhibits pancortin or pancortin-Pablo polypeptide activity or pancortin or pancortin-Pablo nucleic acid expression. These modulatory methods can be performed in vitro (e.g., by culturing the cell with the agent) or, alternatively, in vivo (e.g., by administering the agent to a subject). In a preferred embodiment, the modulatory methods are performed in vivo, i.e., the cell is present within a subject, e.g., a mammal, e.g., a human, and the subject has a disorder or disease characterized by or associated with abnormal or aberrant pancortin or pancortin-Pablo polypeptide activity or pancortin or pancortin-Pablo nucleic acid expression.

A nucleic acid molecule, a protein, a pancortin or pancortin-Pablo modulator, a compound, etc., used in the methods of treatment can be incorporated into an appropriate pharmaceutical composition described below and administered to the subject through a route which allows the molecule, protein, modulator, or compound etc. to perform its intended function.

A modulator of pancortin polynucleotide expression and/or a pancortin polypeptide or a pancortin-Pablo polypeptide dimer activity may be used in the treatment of various diseases or disorders including, but not limited to, the cardiopulmonary system such as acute heart failure, hypotension, hypertension, angina pectoris, myocardial infarction and the like; the gastrointestinal system; the central nervous system; kidney diseases; liver diseases; hyperproliferative diseases, such as cancers and psoriasis; apoptotic diseases; pain; endometriosis; anorexia; bulimia; asthma; osteoporosis; neuropsychiatric disorders such as schizophrenia, delirium, bipolar, depression, anxiety, panic disorders; urinary retention; ulcers; allergies; benign prostatic hypertrophy; and dyskinesias, such as Huntington's disease or Gilles dela Tourett's syndrome

Pharmacogenomics

Test/candidate compounds, or modulators which have a stimulatory or inhibitory effect on pancortin or pancortin-Pablo polypeptide activity (e.g., pancortin or pancortin-Pablo gene expression) as identified by a screening assay described herein can be administered to individuals to treat (prophylactically or therapeutically) disorders (e.g., neurological disorders) associated with aberrant pancortin or pancortin-Pablo polypeptide activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permit the selection of effective compounds (e.g., drugs) for prophylactic or therapeutic treatments based on a consideration of the individual's genotype. Such pharmacogenomics can further be used to determine appropriate dosages and therapeutic regimens. Accordingly, the activity of pancortin or pancortin-Pablo polypeptide, expression of pancortin or pancortin-Pablo nucleic acid, or mutation content of pancortin or pancortin-Pablo genes in an individual can be determined to thereby select appropriate compound(s) for therapeutic or prophylactic treatment of the individual.

Pharmacogenomics deal with clinically significant hereditary variations in the response to drugs due to altered drug disposition and abnormal action in affected persons. See, e.g., Eichelbaum, 1996 and Linder, 1997. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (GOD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2136 and CYP2C 19) has provided an explanation as to why some patients do not obtain the expected drug effects or show exaggerated drug response and serious toxicity after taking the standard and safe dose of a drug.

These polymorphisms are expressed in two phenotypes in the population, the extensive metabolizer (EM) and poor metaboiizer (PM). The prevalence of PM is different among different populations. For example, the gene coding for CYP2136 is highly polymorphic and several mutations have been identified in PM, which all lead to the absence of functional CYP2D6. Poor metabolizers of CYP2136 and CYP2C 19 quite frequently experience exaggerated drug response and side effects when they receive standard doses.

If a metabolite is the active therapeutic moiety, PM show no therapeutic response, as demonstrated for the analgesic effect of codeine mediated by its CYP2136-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of pancortin or pancortin-Pablo polypeptide, expression of pancortin or pancortin-Pablo nucleic acid, or mutation content of pancortin or pancortin-Pablo genes in an individual can be determined to thereby select appropriate agent(s) for therapeutic or prophylactic treatment of a subject. In addition, pharmacogenetic studies can be used to apply genotyping of polymorphic alleles encoding drug-metabolizing enzymes to the identification of a subject's drug responsiveness phenotype. This knowledge, when applied to dosing or drug selection, can avoid adverse reactions or therapeutic failure and thus enhance therapeutic or prophylactic efficiency when treating a subject with a pancortin or pancortin-Pablo modulator, such as a modulator identified by one of the exemplary screening assays described herein.

Monitoring of Effects During Clinical Trials

Monitoring the influence of compounds (e.g., drugs) on the expression or activity of pancortin or pancortin-Pablo polypeptide/gene can be applied not only in basic drug screening, but also in clinical trials. For example, the effectiveness of an agent determined by a screening assay, as described herein, to increase pancortin or pancortin-Pablo gene expression, protein levels, or up-regulate pancortin or pancortin-Pablo activity, can be monitored in clinical trials of subjects exhibiting decreased pancortin or pancortin-Pablo gene expression, protein levels, or down-regulated pancortin or pancortin-Pablo polypeptide activity. Alternatively, the effectiveness of an agent, determined by a screening assay, to decrease pancortin or pancortin-Pablo gene expression, protein levels, or down-regulate pancortin or pancortin-Pablo polypeptide activity, can be monitored in clinical trials of subjects exhibiting increased pancortin or pancortin-Pablo gene expression, protein levels, or up-regulated pancortin or pancortin-Pablo polypeptide activity. In such clinical trials, the expression or activity of a pancortin or pancortin-Pablo polypeptide and, preferably, other genes which have been implicated in, for example, a nervous system related disorder can be used as a “read out” or markers of the ligand responsiveness of a particular cell.

For example, and not by way of limitation, genes, including a pancortin or pancortin-Pablo gene, which are modulated in cells by treatment with a compound (e.g., drug or small molecule) which modulates pancortin or pancortin-Pablo polypeptide/gene activity (e.g., identified in a screening assay as described herein) can be identified. Thus, to study the effect of compounds on CNS disorders, for example, in a clinical trial, cells can be isolated and RNA prepared and analyzed for the levels of expression of a pancortin or pancortin-Pablo gene and other genes implicated in the disorder. The levels of gene expression (i.e., a gene expression pattern) can be quantified by Northern blot analysis or RT-PCR, as described herein, or, alternatively, by measuring the amount of protein produced, by one of the methods described herein, or by measuring the levels of activity of a pancortin or pancortin-Pablo polypeptide or other genes. In this way, the gene expression pattern can serve as an marker, indicative of the physiological response of the cells to the compound. Accordingly, this response state may be determined before, and at various points during, treatment of the individual with the compound.

In a preferred embodiment, the present invention provides a method for monitoring the effectiveness of treatment of a subject with a compound (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the compound; (ii) detecting the level of expression of a pancortin or pancortin-Pablo polypeptide, mRNA, or genomic DNA in the preadministration sample; (iii) obtaining one or more post-administration samples from the subject; (iv) detecting the level of expression or activity of the pancortin or pancortin-Pablo polypeptide, mRNA, or genomic DNA in the post-administration samples; (v) comparing the level of expression or activity of the pancortin or pancortin-Pablo polypeptide, mRNA, or genomic DNA in the pre-administration sample with the pancortin or pancortin-Pablo polypeptide, mRNA, or genomic DNA in the post administration sample or samples; and (vi) altering the administration of the compound to the subject accordingly. For example, increased administration of the compound may be desirable to increase the expression or activity of a pancortin or pancortin-Pablo polypeptide/gene to higher levels than detected, i.e., to increase the effectiveness of the agent.

Alternatively, decreased administration of the agent may be desirable to decrease expression or activity of pancortin or pancortin-Pablo to lower levels than detected, i.e. to decrease the effectiveness of the compound.

Pharmaceutical Compositions

The pancortin or pancortin-Pablo nucleic acid molecules, pancortin or pancortin-Pablo polypeptides (particularly fragments of pancortin or pancortin-Pablo), modulators of a pancortin or pancortin-Pablo polypeptide, and anti-pancortin or pancortin-Pablo antibodies (also referred to herein as “active compounds”) of the invention can be incorporated into pharmaceutical compositions suitable for administration to a subject, e.g., a human. Such compositions typically comprise the nucleic acid molecule, protein, modulator, or antibody and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants, such as ascorbic acid or sodium bisulfite; chelating agents such as ethylenediaminetetraacetic acid; buffers, such as acetates, citrates or phosphates and agents for the adjustment of tonicity, such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a pancortin or pancortin-Pablo polypeptide or anti-pancortin or pancortin-Pablo antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. The pharmaceutical compositions can be included in a container, pack, or dispenser together with instructions for administration.

### EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Identification of Pancortin Interacting Proteins

Yeast Two-Hybrid Assay

Pancortin 2 and pancortin 4 were observed to bind Pablo in yeast two-hybrid assays. Thus, a pancortin protein, a pancortin-Pablo dimer or portions thereof can be used as “bait proteins” in yeast two-hybrid or three-hybrid assays (see, e.g., U.S. Pat. No. 5,283,317 and International Application No. WO 94/10300), to identify other proteins, which bind to or interact with pancortin and/or pancortin-Pablo and are involved in pancortin and/or pancortin-Pablo activity. Such pancortin-binding proteins are also likely to be involved in the propagation of signals by the pancortin proteins or pancortin targets as, for example, downstream elements of a Pablo-mediated signaling pathway. Alternatively, such pancortin-binding proteins may be pancortin inhibitors.

The two-hybrid system is based on the modular nature of most transcription factors, which consist of separable DNA-binding and activation domains. Briefly, the assay utilizes two different DNA constructs. In one construct, the gene that codes for a pancortin protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a pancortin-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the pancortin protein.

### Example 2

### Pancortin Gene Structure

The cDNA sequences of pancortin 1 (SEQ ID NO:1), pancortin 2 (SEQ ID NO:3), pancortin 3 (SEQ ID NO:5), and pancortin 4 (SEQ ID NO:7) each align on Golden Path (Kent et al., in press 2002; Lander et al., 2001) Chromosome 9 (Table 3). In the current draft version of the Golden Path database, the entire Chromosome 9 is represented as a single 131,451,592 base pair (bp) contig (SEQ ID NO:15). The pancortins are found on the forward strand of this contig. The lengths of the sequences are given in parenthesis on the first row of Table 3. Most exon coordinates match perfectly on the genome. Thus, nucleotides 1-150 of pancortin 1 (SEQ ID NO:1), as well as pancortin 2 (SEQ ID NO:3) match up with nucleotides 128764534-128764683 on Chromosome 9. In the cases where the match was not perfect, the genomic coordinate of the imperfect end is given in parenthesis. For example, nucleotides 1-57 of pancortin 4 (SEQ ID NO:7) align with 128752282-128752338 of Chromosome 9. Thus, the genomic sequence of the pancortin gene spans bases 128752282-128796723 of Chromosome 9 of the Golden Path version of Chromosome 9. This 44,442 bp sequence is shown in SEQ ID NO:15.

TABLE 3
Genomic Structure of Pancortins 1-4
as Aligned to Chromosome 9 Sequence in the Golden Path Database

| Chr9 (Golden Path) | Pancortin 1 | Pancortin 2 | Pancortin 3 | Pancortin 4 |
|---|---|---|---|---|
| (131,451,592 bp) | (1458 bp) | (462 bp) | (1374 bp) | (378 bp) |
| 128752282-128752347 | ABSENT | ABSENT | 1-66 | 1-57 |
| | | | | (−128752338) |
| 128764534-128764683 | 1-150 | 1-150 | ABSENT | ABSENT |
| 128766739-128766888 | 151-300 | 151-300 | 67-216 | 58-216 |
| | | | | (128766731−) |
| 128772409-128772564 | 301-456 | 301-462 | 217-372 | 217-378 |
| | | (−128772567) | | (−128772567) |
| 128774831-128775050 | 457-676 | ABSENT | 373-592 | ABSENT |
| 128783294-128783400 | 677-783 | ABSENT | 593-699 | ABSENT |
| 128796049-128796723 | 784-1458 | ABSENT | 700-1374 | ABSENT |

The cDNA sequences of pancortin 1 (SEQ ID NO:1), pancortin 2 (SEQ ID NO:3), pancortin 3 (SEQ ID NO:5), and pancortin 4 (SEQ ID NO:7) each align on the Celera Genomic Axis GA_x54KREBEJAA (Venter et al. 2001) (Table 4). Celera has mapped GA_x54KREBEJAA to bases 109649680-113510886 on the forward strand of chromosome 9. The lengths of the sequences are given in parenthesis on the first row of Table 4. In the cases where the match was not perfect, the genomic coordinate of the imperfect end is given in parenthesis.

TABLE 4
Genomic Structure of Pancortins 1-4 as Aligned to the Celera Database

| GA_x54KREBEJAA | Pancortin 1 | Pancortin 2 | Pancortin 3 | Pancortin 4 |
|---|---|---|---|---|
| (3,861,206 bp) | (1458 bp) | (462 bp) | (1374 bp) | (378 bp) |
| 1811739-1811804 | ABSENT | ABSENT | 1-66 | 1-57 |
| | | | | (−1811795) |
| 1824243-1824392 | 1-150 | 1-150 | ABSENT | ABSENT |
| | | | | 58-216 |
| 1826448-1826597 | 151-300 | 151-300 | 67-216 | (1826440−) |
| 1832155-1832310 | 301-456 | 301-462 | 217-372 | 217-378 |
| | | (−1832313) | | (−1832313) |
| 1834760-1834979 | 457-676 | ABSENT | 373-592 | ABSENT |
| 1843219-1843325 | 677-783 | ABSENT | 593-699 | ABSENT |
| 1855918-1856592 | 784-1458 | ABSENT | 700-1374 | ABSENT |

### Example 3

### Pancortin Homologous Sequences, Expressed Sequence Tags and Single Nucleotide Polymorphisms

Pancortin 1 (SEQ ID NO:1) is highly identical to mouse pancortin 1 (Accession No. D78262; 98%); “olfactomedin-related, ER-localized protein” from Rat (Accession No. U03417; 98%), Gallus gallus (Accession No. AF182815; 96%), Xenopus (Accession No. AF416483; 93%); and a couple of “unknown” proteins from human (Accession No. BC011741; 99% and Accession No. BC008763; 99%). The last two human clones (Accession Nos. BC011741 and BC008763) are in the mammalian gene collection (MGC) at the National Center for Biotechnology Information (NCBI).

In addition to the above high percentage identities, pancortin also shows an intermediate sequence similarity to optimedin form B from mouse (Accession No. AF442824; 66%) and rat (Accession No. AF442822; 66%); optimedin form A from mouse (Accession No. AF442825; 66% ID) and rat (Accession No. AF442823; 64%); and related proteins from human (Accession Nos. AF397392 and AF397394; both 66%). Pancortin 1 also is similar to human olfactomedin 3 (Accession No. BC022531; 65%), to an unknown human MGC clone (Accession No. BC011361; 60%) and to a human olfactomedin related protein which is annotated to be neuronal (Accession No. AF131839; 60% ID). Pancortin 2 (SEQ ID NO:3), pancortin 3 (SEQ ID NO:5) and pancortin 4 (SEQ ID NO:7) show similar hits.

Pancortins 1-4 also have a large number of Expressed Sequence Tag (EST) hits. For example, pancortin 1 (SEQ ID NO:1) hits human ESTs (Accession Nos. BM467174, BI253790, BI253790, BM478361, AU118447, AW957157, BG104648, etc.) and mouse ESTs (Accession Nos. BM949199, BM950765, BM948100, BG342436, BM948052, etc.). Pancortin 2 (SEQ ID NO:3) hits human ESTs (Accession Nos. BI490019, AL533562, BI552459, AV750017, AL533522, etc.) and mouse ESTs (Accession Nos. BG801991, BG807643, BI107666, etc.). Pancortin 3 (SEQ ID NO:5) and pancortin 4 (SEQ ID NO:7) show similar hits.

The pancortin gene was further analyzed for single nucleotide polymorphisms (SNPs) as annotated by Celera in the human genome. Table 6 lists SNPs that occur within or near the pancortin gene. SNP IDs refer to the Celera SNP database. For SNPs outside the gene, the footnotes below the table explain their location.

TABLE 5
SNPs within and near the Pancortin Gene

| Pancortin 1 | | Nucleotide | |
|---|---|---|---|
| Coordinates | SNP ID | Sequence | SNP |
| 1157 | hCV8788652 | C | T |
| 936 | hCV8788651 | C | T |
| 237 | hCV1856773 | C | T |
| Last intron¹ | hCV1856714 | C | A |
| 3p of gene² | hCV1856715 | A | G |
| First intron³ | hCV11569860 | A | — (gap) |
| Last Intron⁴ | hCV1856673 | T | A |
| 2^{nd} last intron⁵ | hCV15877105 | T | C |

¹This is in the last intron, 58 bases upstream of where the last exon begins.
²This is 46 bases 3p of where the gene ends.
³28 bases 3p of the first exon (1-150 nucleotides of pancortin 1)
⁴8 bases into the last intron (8 bases 3p of the 677-783 exon of pancortin 1).
⁵255 bases 5p of the 677-783 exon of pancortin 1.

### Example 4

### Expression of Recombinant Pancortin and Pablo Polypeptide in Bacterial Cells

In this example, pancortin or pancortin and Pablo is/are expressed as a recombinant glutathione-S-transferase (GST) fusion polypeptide in E. coli and the fusion polypeptide is isolated and characterized. Specifically, pancortin or pancortin and Pablo is/are fused to GST and this fusion polypeptide is expressed in E. coli, e.g., strain PEB 199. As the human polypeptide of SEQ ID NO:4 (i.e., pancortin-2) and SEQ ID NO:10 (i.e., Pablo), are predicted to be approximately 17.1 kDa and 61.6 kDa, respectively; and GST is predicted to be 26 kDa, the fusion protein is predicted to be approximately 43.1 kDa and 87.6 kDa, in molecular weight, respectively. Expression of the GST-pancortin and/or GST-Pablo fusion polypeptide in PEB199 is induced with IPTG. The recombinant fusion polypeptide is purified from crude bacterial lysates of the induced PEB 199 strain by affinity chromatography on glutathione beads. Using polyacrylamide gel electrophoretic analysis of the polypeptide purified from the bacterial lysates, the molecular weight of the resultant fusion protein is determined. Alternatively, pancortin may be expressed as a recombinant His-Tag fusion polypeptide using a similar regimen as described above.

### Example 5

### Expression of Recombinant Pancortin and Pablo Polypeptide in COS Cells

To express pancortin or pancortin and Pablo in COS cells, the pcDNA/Amp vector by Invitrogen Corporation (San Diego, Calif.) will be used. This vector contains an SV40 origin of replication, an ampicillin resistance gene, an E. coli replication origin, a CMV promoter followed by a polylinker region, and an SV40 intron and polyadenylation site. A DNA fragment encoding the entire pancortin and Pablo protein and a HA tag (Wilson et al., 1984) fused in-frame to its 3′ end of the fragment is cloned into the polylinker region of the vector, thereby placing the expression of the recombinant protein under the control of the CMV promoter.

To construct the plasmid, the pancortin or pancortin and Pablo DNA sequence is amplified by PCR using two primers. The 5′ primer contains the restriction site of interest followed by approximately twenty nucleotides of the pancortin or pancortin and Pablo coding sequence starting from the initiation codon; the 3′ end sequence contains complementary sequences to the other restriction site of interest, a translation stop codon, the HA tag and the last 20 nucleotides of the pancortin or pancortin and Pablo coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the pancortin or pancortin and Pablo gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the pancortin and/or Pablo-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook et al., “Molecular Cloning: A Laboratory Manual,” 2nd, ed, Cold Spring Harbor Laboratory, Cold Spring Harbor Laboratory Press, Cold Spring Harbor, N.Y., 1989. The expression of the pancortin or pancortin and Pablo polypeptide is detected by radiolabelling (³⁵S-methionine or ³⁵S-cysteine available from NEN, Boston, Mass., can be used) and immunoprecipitation (Harlow and Lane, “Antibodies: A Laboratory Manual,” Cold Spring Harbor Laboratory Press, Cold Spring Harbor, N.Y., 1988) using a HA specific monoclonal antibody. Briefly, the cells are labelled for 8 hours with ³⁵S-methionine (or ³⁵S-cysteine). The culture media are then collected and the cells are lysed using detergents (RIPA buffer, 150 mM NaCl, I %NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the pancortin or pancortin and Pablo coding sequence is cloned directly into the polylinker of the pcDNA/Amp vector using the appropriate restriction sites. The resulting plasmid is transfected into COS cells in the manner described above, and the expression of the pancortin or pancortin and Pablo polypeptide is detected by radiolabelling and immunoprecipitation using an pancortin or pancortin-Pablo specific monoclonal antibody.

### Example 6

### Cell Line Generation

This example describes how to generate a cell line comprising the open reading frame polynucleotide sequence of SEQ ID NO:1, SEQ ID NO:3, SEQ ID NO:5, SEQ ID NO:7 or SEQ ID NO:9. The pancortin or pancortin and Pablo polynucleotide sequence of SEQ ID NO:1, SEQ ID NO:3, SEQ ID NO:5, SEQ ID NO:7 or SEQ ID NO:9 is ligated into the mammalian expression vector pcDNA3.1+zeo (Invitrogen, 1600 Faraday Avenue, Carlsbad, Calif. 92008). CHO cells are transfected with the plasmid and selected with 500 ug/ml zeocin. Zeocin resistant clones are tested for expression of pancortin or pancortin and Pablo by RT-PCR and western blotting. Subsequently, the effects of pancortin or pancortin and Pablo expression on apoptotic signaling is investigated, wherein expression may be inducible via the RU 486 system.

### Example 7

### Construction of a Pancortin Gene Targeting Vector

The identification of pancortins was initially based on cloning of brain-specific transcripts. Subsequently, pancortin was identified as a binding-partner for Pablo, a neuron-specific pro-apoptotic regulator. Differentially processed transcripts are expressed in rat brain in a developmentally and region-specific manner. Four pancortin proteins arise from the usage of two 5′ exons (A and B with independent promoters) along with distinct 3′ exons that encode two different C-termini of the proteins (termini Y and Z) (see FIG. 1, FIG. 2A and FIG. 2B). Matrixing of all combinations result in 4 species of mRNA and protein that share the middle region (M). Pancortin 3 and 4 are the dominant forms during development and may be secreted, while pancortins 1 and 2 predominate during adulthood. Of the four forms, only pancortin 2 appears to functionally bind Pablo.

While several mRNA variants of pancortin have been identified, only pancortin 2 consistently binds Pablo and induces apoptosis in vitro. The C-terminal exon (exon Y) encodes a single amino acid prior to translational stop. Deletion of this exon will lead to loss of pahcortin 2 and 4 and block association with Pablo.

Generation of a pancortin knockout animal will help define the involvement of pancortin in mediating Pablo-induced apoptosis. A Pancortin knock-out animal will facilitate understanding of effect of disrupting Pancortin/Pablo and subsequent protection from apoptotic cell death.

The over expression of Pablo was observed to be toxic in animals such as rats, mice, and in human neuronal cell lines. For example, transgenic mice over expressing Pablo had a phenotype that included tremors, hind limb clasping and death (dependent on the level of Pablo over expression). Transgenic mice having a homozygous Pablo knock-out displayed overt motor dysfunction and post-natal lethality.

Knockout Format. A conventional knockout comprises the deletion of Exon Y, resulting in a knockout of the Pancortin 2 and Pancortin 4 isoforms. Knockout of the Y exon is not expected to result in lethality as the Pancortin 1 and 3 isoforms will be left intact.

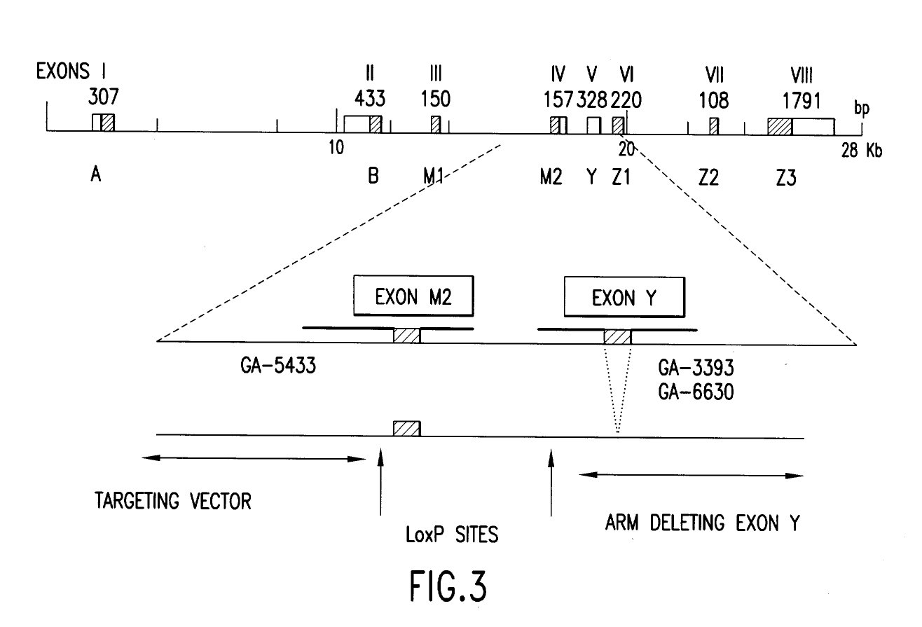

In addition, insertion of LoxP sites flanking Exon M2 will offer a conditional strategy for the knockout of all pancortin species when mated with tissue-specific Cre deleter mice. Insertion of Lox-P sites flanking exon M2 and subsequent excision is expected to obliterate or truncate the expression of proteins to pancortin 1 (BMZ) and pancortin 3 (AMZ). In vitro evidence points to the importance of M1 and M2 for pro-apoptotic function. Thus, animals bearing the deletion of M2 would be useful to compare the effects of Exon Y deletion (specific to pancortin2 and pancortin4) to the functional deletion of all pancortin subtypes. FIG. 3 presents a schematic for preparing a targeting vector for pancortin knockout.

Phenotypic characterization of pancortin knockout animals may be performed in combination, with primary neuronal cultures preceding in vivo experiments involving acute ischemic or pro-apoptotic insult.

### Example 8

### Transfection and Analysis Of Embryonal Stem Cells

Embryonic stem cells (e.g., strain D3, Doestschman, et al. 1985) are cultured on a neomycin resistant embryonal fibroblast feeder layer grown in Dulbecco's Modified Eagles medium supplemented with 15% Fetal Calf Serum, 2 mM glutamine, penicillin (50 u/ml)/streptomycin (50 u/ml), non-essential amino acids, 100 uM 2-mercaptoethanol and 500 u/ml leukemia inhibitory factor. Medium is changed daily and cells are subcultured every two to three days and are then transfected with linearized plasmid by electroporation (25 uF capacitance and 400 Volts). The transfected cells are cultured in non-selective media for 1-2 days post transfection. Subsequently, they are cultured in media containing gancyclovir and neomycin for 5 days, of which the last 3 days are in neomycin alone. After expanding the clones, an aliquot of cells is frozen in liquid nitrogen. DNA is prepared from the remainder of cells for genomic DNA analysis to identify clones in which homologous recombination had occurred between the endogenous pancortin and/or Pablo gene and the targeting construct. To prepare genomic DNA, ES cell clones are lysed in 100 mM Tris HCl, pH 8.5, 5 mM EDTA, 0.2% SDS, 200 mM NaCl and 100 ug of proteinase K/ml. DNA is recovered by isopropanol precipitation, solubilized in 10 mM Tris HCl, pH 8.0/0.1 mM EDTA. To identify homologous recombinant clones, genomic DNA isolated from the clones is digested with restriction enzymes. After restriction digestion, the DNA can be resolved on a 0.8% agarose gel, blotted onto a Hybond N membrane and hybridized at 65° C. with probes that bind a region of the pancortin or pancortin and Pablo gene proximal to the 5′ end of the targeting vector and probes that bind a region of the pancortin or pancortin and Pablo gene distal to the 3′ end of the targeting vector. After standard hybridization, the blots are washed with 40 mM NaPO4 (pH 7.2), 1 mM EDTA and 1% SDS at 65° C. and exposed to X-ray film. Hybridization of the 5′ probe to the wild type pancortin or pancortin and Pablo allele results in a fragment readily discernible by autoradiography from the mutant pancortin or pancortin and Pablo allele having the neo insertion.

### Example 9

### Generation of Pancortin and Pablo Deficient Mice

Female and male mice are mated and blastocysts are isolated at 3.5 days of gestation. 10 to 12 cells from the clone described in Example 2 are injected per blastocyst and 7 or 8 blastocysts are transferred to the uterus of a pseudopregnant female. Pups are delivered by cesarean section on the 18th day of gestation and placed with a foster BALB/c mother. Resulting male and female chimeras are mated with female and male BALB/C mice (non-pigmented coat), respectively, and germline transmission is determined by the pigmented coat color derived from passage of 129 ES cell genome through the germline. The pigmented heterozygotes are likely to carry the disrupted pancortin and/or Pablo allele and therefore these animals are mated and, Mendelian genetics predicts that approximately 25% of the offspring will be homozygous for the pancortin and/or Pablo null mutation. Genotyping of the animals is accomplished by obtaining tail genomic DNA.

To confirm that the pancortin and/or Pablo −/−mice do not express full-length pancortin and/or Pablo mRNA transcripts, RNA is isolated from various tissues and analyzed by standard Northern hybridizations with an pancortin and/or Pablo cDNA probe or by reverse transcriptase-polymerase chain reaction (RT-PCR). RNA is extracted from various organs of the mice using 4M Guanidinium thiocyanate followed by centrifugation through 5.7 M CsCl as described in Sambrook et al. (Molecular Cloning: A Laboratory Manual, 2nd Edition, Cold Spring Harbor Laboratory press (1989)). Northern analysis of mRNA isolated from pancortin and/or Pablo expressing tissues will demonstrate that the full-length pancortin and/or Pablo mRNA is not detectable in pancortin and/or Pablo −/− mice. Primers specific for the neomycin gene will detect a transcript in pancortin and/or Pablo +/− and −/− but not +/+ animals. Northern and RT-PCT analyses are used to confirm that homozygous disruption of the pancortin and/or Pablo gene results in the absence of detectable full-length pancortin and/or Pablo mRNA transcripts in the pancortin and/or Pablo −/− mice. To examine pancortin and/or Pablo protein expression in the pancortin and/or Pablo deficient mice, Western blot analyses are performed on lysates from isolated tissue using standard techniques. These results will confirm that homozygous disruption of the pancortin and/or Pablo gene results in an absence of detectable pancortin and/or Pablo protein in the −/− mice.

### Example 10

### Inhibition of Pancortin and/or Pablo Production

Design of RNA Molecules as Compositions of the Invention

All RNA molecules in this experiment are approximately 600 nts in length, and all RNA molecules are designed to be incapable of producing functional pancortin and/or Pablo protein. The molecules have no cap and no poly-A sequence; the native initiation codon is not present, and the RNA does not encode the full-length product. The following RNA molecules are designed:

(1) a single-stranded (ss) sense RNA polynucleotide sequence homologous to a portion of pancortin and/or Pablo murine messenger RNA (mRNA);

(2) a ss anti-sense RNA polynucleotide sequence complementary to a portion of pancortin and/or Pablo murine mRNA,

(3) a double-stranded (ds) RNA molecule comprised of both sense and anti-sense portion of pancortin and/or Pablo murine mRNA polynucleotide sequences,

(4) a ss sense RNA polynucleotide sequence homologous to a portion of pancortin and/or Pablo murine heterogeneous RNA (hnRNA),

(5) a ss anti-sense RNA polynucleotide sequence complementary to a portion of pancortin and/or Pablo murine hnRNA,

(6) a ds RNA molecule comprised of the sense and anti-sense pancortin and/or Pablo murine hnRNA polynucleotide sequences,

(7) a ss murine RNA polynucleotide sequence homologous to the top strand of the portion of pancortin and/or Pablo promoter,

(8) a ss murine RNA polynucleotide sequence homologous to the bottom strand of the portion of pancortin-Pablo promoter, and

(9) a ds RNA molecule comprised of murine RNA polyriucleotide sequences homologous to the top and bottom strands of the pancortin and/or Pablo promoter.

The various RNA molecules of (1)-(9) above may be generated through T7 RNA polymerase transcription of PCR products bearing a T7 promoter at one end. In the instance where a sense RNA is desired, a T7 promoter is located at the 5′ end of the forward PCR primer. In the instance where an anti-sense RNA is desired, the T7 promoter is located at the 5′ end of the reverse PCR primer. When dsRNA is desired both types of PCR products may be included in the T7 transcription reaction. Alternatively, sense and anti-sense RNA may be mixed together after transcription.

Construction of Expression Plasmid Encoding a Fold-Back Type of RNA

Expression plasmid encoding an inverted repeat of a portion of the pancortin and/or Pablo gene may be constructed using the information disclosed in this application. Two pancortin and/or Pablo gene fragments of approximately at least 600 nucleotides in length, almost identical in sequence to each other, may be prepared by PCR amplification and introduced into a suitable restriction of a vector which includes the elements required for transcription of the pancortin and/or Pablo fragment in an opposite orientation. CHO cells transfected with the construct will produce only fold-back RNA in which complementary target gene sequences form a double helix. The genomic and PCR primer coordinates are based on the sequence of SEQ ID NO: 1, SEQ ID NO: 3, SEQ ID NO:5, SEQ ID NO:7 or SEQ ID NO:9.

Assay

Balb/c mice (5 mice/group) may be injected intramuscularly or intraperitoneally with the murine pancortin and/or Pablo chain specific RNAs described above or with controls at doses ranging between 10 pg and 500 pg. Sera is collected from the mice every four days for a period of three weeks and assayed for pancortin and/or Pablo levels using the antibodies as disclosed herein.

### Example 11

### Method of the Invention in the Prophylaxis of Disease

In Vivo Assay

Using the pancortin and/or Pablo specific RNA molecules described above, which do not have the ability to make pancortin and/or Pablo protein and pancortin and/or Pablo specific RNA molecules as controls, mice may be evaluated for protection from pancortin and/or Pablo related disease through the use of the injected pancortin and/or Pablo specific RNA molecules of the invention. Balb/c mice (5 mice/group) may be immunized by intercranial injection with the described RNA molecules at doses ranging between 10 and 500 μg RNA. At days 1, 2, 4 and 7 following RNA injection, the mice may be observed for signs of pancortin and/or Pablo related phenotypic change.

According to the present invention, because the mice that receive dsRNA molecules of the present invention which contain the pancortin and/or Pablo sequence may be shown to be protected against pancortin and/or Pablo related disease. The mice receiving the control RNA molecules may not be protected. Mice receiving the ss RNA molecules which contain the pancortin and/or Pablo sequence may be expected to be minimally, if at all, protected, unless these molecules have the ability to become at least partially double stranded in vivo.

According to this invention, because the dsRNA molecules of the invention do not have the ability to make pancortin and/or Pablo protein, the protection provided by delivery of the RNA molecules to the animal is due to a non-immune mediated mechanism that is gene specific.

### Example 12

### RNA Interference in Drosophila and Chinese Hamster Cultured Cells

To observe the effects of RNA interference, either cell lines naturally expressing pancortin and/or Pablo can be identified and used or cell lines which express pancortin and/or Pablo as a transgene can be constructed by well known methods (and as outlined herein). As examples, the use of Drosophila and CHO cells are described. Drosophila S2 cells and Chinese hamster CHO-K1 cells, respectively, may be cultured in Schneider medium (Gibco BRL) at 25° C. and in Dulbecco's modified Eagle's medium (Gibco BRL) at 37° C. Both media may be supplemented with 10% heat-inactivated fetal bovine serum (Mitsubishi Kasei) and antibiotics (10 units/ml of penicillin (Meiji) and 50 μg/ml of streptomycin (Meiji)).

Transfection and RNAi Activity Assay

S2 and CHO-K1 cells, respectively, are inoculated at 1×10⁶ and 3×10⁵ cells/ml in each well of 24-well plate. After 1 day, using the calcium phosphate precipitation method, cells are transfected with pancortin and/or Pablo dsRNA (80 pg to 3 μg). Cells may be harvested 20 hours after transfection and pancortin and/or Pablo gene expression measured.

### Example 13

### Antisense Inhibition In Vertebrate Cell Lines

Antisense can be performed using standard techniques including the use of kits such as those of Sequitur Inc. (Natick, Mass.). The following procedure utilizes phosphorothioate oligodeoxynucleotides and cationic lipids. The oligomers are selected to be complementary to the 5′ end of the mRNA so that the translation start site is encompassed.

1) Prior to plating the cells, the walls of the plate are gelatin coated to promote adhesion by incubating 0.2% sterile filtered gelatin for 30 minutes and then washing once with PBS. Cells are grown to 40-80% confluence. Hela cells can be used as a positive control.

2) the cells are washed with serum free media (such as Opti-MEMA from Gibco-BRL).

3) Suitable cationic lipids (such as Oligofectibn A from Sequitur, Inc.) are mixed and added to serum free media without antibiotics in a polystyrene tube. The concentration of the lipids can be varied depending on their source. Add oligomers to the tubes containing serum free media/cationic lipids to a final concentration of approximately 200 nM (50-400 nM range) from a 100 μM stock (2 μl per ml) and mix by inverting.

4) The oligomer/media/cationic lipid solution is added to the cells (approximately 0.5 mls for each well of a 24 well plate) and incubated at 37° C. for 4 hours.

5) The cells are gently washed with media and complete growth media is added. The cells are grown for 24 hours. A certain percentage of the cells may lift off the plate or become lysed. Cells are harvested and pancortin and/or Pablo gene expression is measured.

### Example 14

### Identification of Pancortin and/or Pablo Binding Proteins and Agonists/Antagonists

Yeast strains, bacterial strains and media for yeast and bacterial selections and growth are well known in the art (see e.g., Klein et al., 1989(a), 1989b; Bartel et al., 1993(b)), as are plating procedures (Rose et al., 1990). A pancortin and/or Pablo polypeptide of the invention is expressed as a fusion protein (‘bait’) in the binding domain portion of the GAL4 protein in the pAS2-1 vector. A human brain library is then expressed in the form of fusions (prey) to the activation domain portion of the GAL4 protein in the pACT II vector. Functional interaction of pancortin and/or Pablo with a library protein will drive the expression of the reporter gene activity. The reporter phenotypes to be utilized are histidine prototrophy and beta-galactosidase activity. The pancortin and/or Pablo used as bait will be the human cDNA from the start codon to stop codon of SEQ ID NO:1, SEQ ID NO:3, SEQ ID NO:5, SEQ ID NO:7 or SEQ ID NO:9. Protein interactions identified as described above, may further be screened with ligands, wherein the ligand may attenuate the protein-protein interaction, or alternatively, the ligand may induce a protein-protein interaction, not detected in the absence of the ligand.

### Example 15

### Assays

Cells expressing pancortin and/or Pablo can be used to screen for compounds which increase (agonists) or decrease (antagonists) the effects of the pancortin-Pablo polypeptide dimer. The effects of test compounds can be screened in functional assays in which a pancortin-Pablo dimer modulates the signaling of apoptosis which can be detected by binding assays, ligand binding assays, mammalian two-hybrid assays, or assays which use an apoptotic endpoint as a readout (for example, propidium iodide uptake, tunnel staining, annexin staining, mitochondrial membrane potential dyes).

Also, a yeast two-hybrid system like the one described can be used to screen for compounds which increase or decrease pancortin-Pablo binding. Changes in growth or expression of a reporter gene (e.g. luciferase) can be used to assay effects of agonists or antagonists of pancortin-Pablo binding.

Recombinantly expressed pancortin and Pablo proteins, or fragments thereof, can be used in an ELISA type format for a cell-free type of screen. For example, a His-tagged Pablo is bound to a nickel coated well of a screening plate (e.g. 96 or 384 well plate). A GST- or thioredoxin tagged pancortin is added to the well and unbound pancortin is washed away. Pancortin binding to Pablo is quantified by immunodetection of the bound GST or thioredoxin tag. The ability of the agonists or antagonists to increase or decrease this binding can be quantified.

### Example 16

### Pablo-Pancortin Co-Immunoprecipitation

Co-immunoprecipitation studies demonstrate that endogenous Pablo protein and endogenous pancortin protein bind to each other in adult rat brain cortex. Co-immunoprecipitation studies were done following standard, published protocols.

Endogenous Co-Immunoprecipitation Protocol

A 1 ml Wheaton glass homogenizer was filled with 1 ml of either Mild Lysis Buffer (CytoSignal: IMMUNOcatcher buffer kit, cat# C04-050) or strong lysis buffer (0.5% SDS; 50 mM Tris-HCl, pH 7.5; 10% glycerol; 1% TritonX-100; 150 mM NaCl; 5 mM EDTA) containing 1 Roche Complete tablet, cat#1 836 170 and 4 mM of Roche Pefabloc SC (AEBSF), cat#104290876 per 10 ml of buffer. All apparatus and buffers were kept at 4° C. Cortex was removed from an adult rat brain, placed immediately in the buffer and homogenized on ice using 8 strokes of the glass pestle or until there is no visible tissue bits. For protein assay, Pierce BCA protein assay reagents, cat#23223, 23224 were used.

To pre-clear the lysate, 50 ul each of Roche protein A-agarose cat#101340515 and protein G-agarose, cat#102430233 were added to 2 mg of protein. The volume was brought to 750 ul using corresponding lysis buffer containing protease inhibitors. The mixture was then incubated in a rotating shaker for 3-5 hours, at 4° C.; centrifuged at 10,000×g for 1 minute in a refrigerated microfuge to remove the nonspecifically absorbed and insoluble material and the pre-cleared supernatant was collected. Using 500 ug total protein for each immunoprecipitation, 5 ug of PABLO monoclonal 33.1; PABLO polyclonal 15053; or Pancortin monoclonal 7.1 was added to each reaction and incubated at 4° C., overnight, in a rotating shaker. 60 ul of protein G-agarose was then added to the tubes with mouse IgG1 monoclonal antibodies added previously and protein A to the tubes with polyclonal antibodies. Incubate at 4° C., for 1-2 hours, in a rotating shaker and then pellet the beads by centrifuging at 10,000×g for 1 minute, at 4° C. The supernatant was removed, the beads resuspended in 1 ml of lysis buffer without protease inhibitors and the washing was repeated 2 more times.

Subsequently, 2× gel loading buffer was added to the final pellet using Invitrogen NuPage LDS sample buffer, cat# NP0007 with 20% NuPage reducing agent, cat# NP0004 to the pellet. The protein was denatured by vortexing and heating at 95° C. for 5 minutes. Protein A or G-Agarose beads were removed by centrifuge at 10,000×g for 3 minutes, at room temperature. The supernatant was then analyzed by SDS-polyacrylamide gel electrophoresis, using NuPage pre-cast gel, NuPage buffer system and antioxidant, cat# NP0005.

A membrane was pre-wet in methanol for 5 seconds, placed in transfer buffer (lx NuPage transfer buffer, cat# NP0006; 20% methanol), and the gel was transferred onto a Millipore Immobilon-P membrane, cat# IPVH07850 by standard western blotting. The western blot was probed with Pablo or pancortin antibody. Polyclonal antibody was used if a monoclonal antibody was used for immunoprecipitation of Pablo and vice versa. Only monoclonal antibody was used to probe pancortin. The blot was then incubated at room temperature, on a rocking platform for 2 hours, and then washed 3 times with Gibco PBS with 0.1% Tween-20, 5 minutes each time, and 1 time for 15 minutes.

The washed blots were the incubated with secondary antibody, HRP conjugated donkey anti rabbit (Jackson ImmunoResearch 715-035-152) for polyclonal primary antibody and HRP conjugated donkey anti mouse antibody (Jackson ImmunoResearch 715-035-150) for monoclonal primary antibody and rock on a rocking platform for 1 hour at room temperature. The blots are washed again as described above after primary antibody incubation.

Add Amersham Biosciences ECL-plus, cat# RPN 2132 onto the surface of the blot, incubate for 5 minutes, at room temperature. The signal was detected using a Phosphoimager (Molecular Dynamics Storm 860), blue fluorescence/chemifluorescence scanner, voltage set at 750 PMT.

TABLE 6

| | | Strong | |
|---|---|---|---|
| Immunoprecipation | Western Blot | lysis buffer | Mild lysis buffer |
| Pablo | Pancortin | − | + |
| Pancortin | Pablo | − | + |

Table 6 above shows that under mild lysis conditions, immunoprecipitation of Pablo also precipitates Pancortin protein, and vice versa. The inability of Pablo and pancortin to co-immunoprecipitate in the presence of strong lysis conditions may be reflective of a weaker protein-protein interaction, or possibly is reflective of properties of the Pablo and pancortin antibodies.

### Example 17

### Rat Middle Cerebral Artery Occlusion Model of Stroke

Adult male Wistar rats (Charles River, Wilmington, Mass.) 290-310 g were anesthetized with 3% isoflurane in 70% nitrous oxide and 30% oxygen through a nose cone. Temperature was maintained at 37° C. throughout the surgery using a heating lamp. Transient middle cerebral artery occlusion (MCAO) was induced for 90 minutes using the intraluminal suture method (Longa et al., 1989).

Briefly, an 18 mm length of 4-0 monofilament nylon suture coated with poly-L-lysine (Belayev et al., 1996) and a flame-rounded tip was inserted into the external carotid artery and advanced through the internal carotid to occlude the origin of the middle cerebral artery (MCA). Ninety minutes later the rats were re-anesthetized and the suture was withdrawn. Sham operated controls were subject to the same surgery but without advancement of the suture into the MCA.

TABLE 7
Pablo-Pancortin Co-Immunoprecipitation time course
following MCAO
Time post-ischemia (days)

| | Sham | 0 | 0.125 | 0.25 | 1 | 3 | 5 | 7 | 11 |
|---|---|---|---|---|---|---|---|---|---|
| Ipsilateral | + | + | + + | + + + | + + + + | − | − | + | + |
| cortex | | | | | | | | | |
| Contralateral | + | + | + + | + + + | + + + + | + + | + | + | + |
| cortex | | | | | | | | | |

There is an increase in Pablo and pancortin complex formation during the reperfusion period of injury. Table 17 above shows the amount of pancortin immunoprecipitated with anti-Pablo antibody reaches its peak by 24 hours post-ischemia. The levels drop dramatically, to below sham levels, on the ipsilateral side by 3 days and remains depressed until approximately 7 days post-ischemia. The contralateral side shows an increase in Pablo-pancortin complex similar to the ipsilateral side. However, this complex does not incorporate Bcl-xL as does the ipsilateral side, and there is no significant neuronal loss in the contralateral cortex. Also, unlike the ipsilateral side, the level of Pablo-pancortin interaction on the contralateral side slowly returns to Sham levels after the rise in the first 24 hours post-injury.

This data provides evidence that a Pablo-pancortin complex forms during the reperfusion period of the rat MCAO model of stroke. The timing of the complex formation precedes the period of significant neuronal loss and therefore may contribute to it. The rapid drop in complex formation on the ipsilateral cortex may be a result of the neurons activating the actin interacting properties of Pablo and thus may be indicative that disrupting the Pablo-pancortin complex will also be beneficial during the recovery period.

Equivalents: Those skilled in the art will recognize, or be able to ascertain using no more than routine experimentation, many equivalents to the specific embodiments of the invention described herein. Such equivalents are intended to be encompassed by the following claims.

### REFERENCES

European Application No. EP 125,023

European Application No. EP 171,496

European Application No. EP 184,187

European Application No. EP 264,166

U.S. Pat. No. 4,196,265

U.S. Pat. No. 4,522,811

U.S. Pat. No. 4,554,101

U.S. Pat. No. 4,683,195

U.S. Pat. No. 4,683,202

U.S. Pat. No. 4,736,866

U.S. Pat. No. 4,816,567

U.S. Pat. No. 4,870,009

U.S. Pat. No. 4,873,191

U.S. Pat. No. 4,873,316

U.S. Pat. No. 4,987,071

U.S. Pat. No. 5,116,742

U.S. Pat. No. 5,223,409

U.S. Pat. No. 5,272,057

U.S. Pat. No. 5,283,317

U.S. Pat. No. 5,328,470

U.S. Pat. No. 5,498,531

U.S. Pat. No. 5,766,844

U.S. Pat. No. 5,789,654

U.S. Pat. No. 5,798,209

U.S. Pat. No. 6,054,297

U.S. Pat. No. 6,201,103

U.S. Statutory Invention Registration No. H1,892

International Application No. WO 00/63364

International Application No. WO 86/01533

International Application No. WO 90/02809

International Application No. WO 90/11354

International Application No. WO 91/01140

International Application No. WO 91/17271

International Application No. WO 92/01047

International Application No. WO 92/09690

International Application No. WO 92/15679

International Application No. WO 92/18619

International Application No. WO 92/20791

International Application No. WO 93/01288

International Application No. WO 93/04169

International Application No. WO 94/10300

International Application No. WO 94/16101

International Application No. WO 97/07668

International Application No. WO 97/07669

Abravaya et al., Nucleic Acids Res., 23:675-682, 1995.

Adams et al., Nature 355:632-634, 1992.

Adams et al., Nature 377 Supp:3-174, 1995.

Adams et al., Science 252:1651-1656, 1991.

Altschul et al., J. Molec. Biol. 215:403-410, 1990.

Amann et al., Gene 69:301-315, 1988.

Bakhshi et al., Cell 41:889-906, 1985.

Baldari et al., Embo J. 6:229-234, 1987.

Banerji et al., Cell, 33:729-740; 1983.

Barembaum et al., “Noelin-1 is a secreted glycoprotein involved in generation of the neural crest” Nature Cell Biology, 2: 219-225, 1999.

Bartel and Szostak, Science 261:1411-1418, 1993.

Bartel et al. Biotechniques 14:920-924, 1993(b).

Bartel, “Cellular Interactions and Development: A Practical Approach”, pp. 153-179, 1993(a).

Belayev, Alonso, Busto, Zhao and Ginsberg, “Middle cerebral artery occlusion in the rat by intraluminal suture: neurological and pathological evaluation of an improved model”, Stroke 27:1616-1623, 1986.

Bradley, Current Opinion in Biotechnology 2:823-829, 1991.

Bradley, in “Teratocarcinomas and Embryonic Stem Cells: A Practical Approach,” E. J. Robertson, ed., IRL, Oxford, pp. 113-152, 1987.

Bunzow et al., Nature, 336:783-787, 1988.

Burge and Karlin, “Prediction of complete gene structures in human genomic DNA.” J. Mol. Biol. 268:78-94, 1997.

Byrne and Ruddle, PNAS 86:5473-5477, 1989.

Calame and Eaton, Adv. Immunol. 43:235-275, 1988.

Campes and Tilghman, Genes Dev. 3:537-546, 1989.

Chen et al., PNAS 91:3054-3057, 1994.

Chittenden et al., Embo J. 14:5589-5596, 1995.

Cleary and Sklar, Proc Natl Acad Sci USA 82:7439-7443, 1985.

Cohen et al., Adv. Chromatogr. 36:127-162, 1996.

Cotton et al., PNAS 85:4397, 1988.

Cotton, Mutat. Res. 285:125-144, 1993.

Danielson et al., “Four structurally distinct neuron-specific olfactomedin-related glycoproteins produced by differential promoter utilization and alternative mRNA splicing from a single gene” Journal of Neuroscience Research, 38(4):468-78, 1994.

D'Eustachio et al., Science 220:919-924, 1983.

Devereux et al., Nucleic Acids Research 12(1):387, 1984.

Doestschman et al., J. Embryol. Exp. Morphol. 87:27-45, 1985.

Edlund et al., Science 230:912-916, 1985.

Eichelbaum, Clin. Exp. Pharmacol Physiol, 23(10-11):983-985, 1996.

Elledge et al., Proc. Natl. Acad. Sci. USA, 88:1731-1735, 1991.

Fan et al., PNAS, 87:6223-27, 1990.

Farrow et al., Curr. Opin. Gen. Dev. 6: 45-49, 1996.

Finely et al., Proc. Natl. Acad. Sci. USA, 91:12980-12984, 1994.

Frohman et al., Proc. Natl. Acad. Sci. USA 85, 8998-9002, 1988.

Gaultier et al., Nucleic Acids Res. 15:6625-6641, 1987.

Gentz et al., Proc. Natl. Acad. Sci. USA, 86:821-824, 1989.

Griffin et al., Appl. Biochem. Biotechnol. 38:147-159, 1993.

Grutter, “Caspases: key players in programmed cell death,” Curr. Opinion in Struct. Biol., 10:649-655, 2000.

Gunnar von Heijne, “Membrane Protein Structure Prediction, Hydrophobicity Analysis and the Positive-inside Rule” J. Mol. Biol., 225:487-494, 1992.

Harlow and Lane, “Antibodies: A Laboratory Manual,” Cold Spring Harbor Laboratory Press, Cold Spring Harbor, N.Y., 1988.

Harper et al., Cell, 75:805-816, 1993.

Haselhoff and Gerlach, Nature 334:585-591, 1988.

Hayashi, Genet. Anal. Tech. Appl. 9:73-79, 1992.

Helene et al., Ann. N. Y Acad Sci. 660:27-36, 1992.

Helene, Anticancer Drug Des. 6(6):569-84, 1991.

Hogan, “Manipulating the Mouse Embryo,” Cold Spring Harbor Laboratory Press, Cold Spring Harbor, N.Y., 1986.

Inoue et al., FEBS Lett. 215:327-330, 1987(a).

Inoue et al., Nucleic Acids Res. 15:6131-6148, 1987(b).

Iwabuchi et al., Oncogene 8:1693-1696, 1993.

Johnson et al., Endoc. Rev., 10:317-331, 1989.

Karavanich and Anholt, “Evolution of olfactomedin. Structural constraints and conservation of primary sequence motifs” Annals of the New York Academy of Sciences, 855:294-300, 1998.

Karavanich and Anholt, “Molecular evolution of olfactomedin” Molecular Biology & Evolution, 15(6):718-26, 1998.

Kaufman et al., EMBO J 6:187-195, 1987.

Kent et al., “The Human Genome Browser at U.C.S.C.”, Genome Research, in press 2002.

Kessel and Gruss, Science 249:374-379, 1990.

Klein et al., Curr. Genet., 16:145-152, 1989(b).

Klein et al., Curr. Genet. 13:29-35, 1989(a).

Kondo et al., “Localization of olfactomedin-related glycoprotein isoform (BMZ) in the golgi apparatus of glomerular podocytes in rat kidneys” Journal of the American Society of Nephrology, 11 (5):803-13, 2000.

Korsmcyer, Blood 80:879-886, 1992.

Kulkarni et al., “Characterization and differential expression of a human gene family of olfactomedin-related proteins” Genetica Research, 76(1):41-50, 2000.

Kurj an and Herskowitz, Cell 933-943, 1982.

Kyte and Doolittle, J. Mol. Biol., 157:105-132, 1982.

Lakso et al., PJVAS 89:6232-6236, 1992.

Lander et al., “Initial Sequencing and Analysis of the Human Genome”, Nature 409:860-921, 2001.

Lefkowitz, Nature, 351:353-354, 1991.

Li et al., Cell 69:915, 1992.

Linder, Clin. Chem. 43(2):254-266, 1997.

Lucklow and Summers, Virology 170:31-39, 1989.

Madura et al., J. Biol. Chem. 268:12046-1205, 1993

Maher, Bioassays 14(12):807-15, 1992.

Mansour et al., Nature 336:348, 1988

Maxim and Gilbert, PNAS 74:560, 1977.

Morin et al., Nucleic Acids Res., 21:2157-2163, 1993.

Muchmore et al., Nature 381:335-341, 1996.

Myers et al., Nature 313:495, 1985(a).

Myers et al., Science 230:1242, 1985(b).

Nagano et al., “A2-Pancortins (Pancortin-3 and -4) are the dominant pancortins during neocortical development” Journal of Neurochemistry, 75(1):1-8, 2000.

Nagano et al., “Differentially expressed Olfactomedin-related glycoproteins (Pancortins) in the brain,” Mol. Brain Res. 53:12-23, 1998.

O'Gonnan et al., Science 251:1351-1355, 1991.

Oltvai and Korsmeyer, Cell 79:189-192, 1994.

Oltvai et al., Cell 74:609-619, 1992.

Orita et al., PNAS 86:2766, 1989.

Pinkert et al. Genes Dev. 1:268-277, 1987.

Rahman et al., Journal of Neuroscience 19:2016-2026, 1999.

Rose et al., “Methods in Yeast Genetics: A Laboratory Course Manual.” Cold Spring Harbor Press, Cold Spring Harbor, N.Y. (1990).

Saleeba et al., Meth. Enzymol. 217:286-295, 1992.

Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.

Sanger, PNAS 74:5463, 1977.

Schultz et al., Gene 54:113-123, 1987.

Seed, Nature 329:840, 1987.

Simon et al., Science, 252:802-8, 1991.

Smith and Johnson, Gene 67:31-40, 1988.

Smith et al., Mol. Cell Biol. 3:2156-2165, 1983.

Songyang, et al., Cell 72:767-778, 1993.

Studier et al. “Gene Expression Technology” Methods in Enzymology 185, 60-89, 1990.

Thomas and Capecchi, Cell 51:503, 1987.

Tsujimoto et al., Science 229:1390-1393, 1985.

Venter et al., “The Sequence of the Human Genome”, Science 291:1204-1351, 2001.

Wilmut et al., Nature 385:810-813, 1997.

Wilson et al., Cell 37:767, 1984.

Winoto and Baltimore. EMBO J 8:729-733, 1989.

Yin et al., Nature 369:321-323, 1994.

Zea Longa et al., “Reversible middle cerebral artery occlusion without craniectomy in rats,” Stroke 20:294-297, 1989.

Zervos et al., Cell 72:223-232, 1993.

What is claimed is:

1. An isolated polynucleotide encoding a human pancortin polypeptide, wherein the polynucleotide comprises a nucleotide sequence having at least 95% identity to the nucleotide sequence of SEQ ID NO:1, a degenerate variant thereof or a complement thereof.

2. The polynucleotide of claim 1, wherein the polynucleotide comprises the nucleotide sequence of SEQ ID NO:1, a degenerate variant thereof or a complement thereof.

3. An isolated polynucleotide encoding a human pancortin polypeptide, wherein the polynucleotide comprises a nucleotide sequence having at least 95% identity to the nucleotide sequence of SEQ ID NO:3, a degenerate variant thereof or a complement thereof.

4. The polynucleotide of claim 3, wherein the polynucleotide comprises the nucleotide sequence of SEQ ID NO:3, a degenerate variant thereof or a complement thereof.

5. An isolated polynucleotide encoding a human pancortin polypeptide, wherein the polynucleotide comprises a nucleotide sequence having at least 95% identity to the nucleotide sequence of SEQ ID NO:5, a degenerate variant thereof or a complement thereof.

6. The polynucleotide of claim 5, wherein the polynucleotide comprises the nucleotide sequence of SEQ ID NO:5, a degenerate variant thereof or a complement thereof.

7. An isolated polynucleotide encoding a human pancortin polypeptide, wherein the polynucleotide comprises the nucleotide sequence of SEQ ID NO:7 a degenerate variant thereof or a complement thereof.

8. The polynucleotide according to claims 1, 3, 5 or 7, wherein the polynucleotide is selected from the group consisting of DNA, cDNA, RNA and antisense RNA.

9. The polynucleotide of claim 8, further comprising heterologous nucleotides.

10. The polynucleotide of claim 1, wherein the polynucleotide encoding a polypeptide comprises an amino acid sequence of SEQ ID NO: 2, a variant thereof or a fragment thereof.

11. The polynucleotide of claim 3, wherein the polynucleotide encoding a polypeptide comprises an amino acid sequence of SEQ ID NO: 4, a variant thereof or a fragment thereof.

12. The polynucleotide of claim 5, wherein the polynucleotide encoding a polypeptide comprises an amino acid sequence of SEQ ID NO: 6, a variant thereof or a fragment thereof.

13. The polynucleotide of claim 7, wherein the polynucleotide encoding a polypeptide comprises an amino acid sequence of SEQ ID NO: 8, a variant thereof or a fragment thereof.

14. The polynucleotide according to claims 10, 11, 12 or 13, wherein the polypeptide binds a pablo polypeptide comprising the amino acid sequence of SEQ ID NO: 9, a variant thereof, or a fragment thereof.

15. The polynucleotide according to claims 10, 11, 12 or 13, wherein the polypeptide is a fusion polypeptide.

16. An isolated polynucleotide which hybridizes with a polynucleotide comprising the nucleotide sequence of SEQ ID NO: 1, SEQ ID NO: 3, SEQ ID NO: 5, SEQ ID NO: 7 or a complement thereof, under high stringency hybridization conditions.

17. An isolated human pancortin polypeptide encoded by a polynucleotide comprising a nucleotide sequence having at least 95% identity to the nucleotide sequence of SEQ ID NO:1, a degenerate variant thereof or a complement thereof.

18. An isolated human pancortin polypeptide encoded by a polynucleotide comprising a nucleotide sequence having at least 95% identity to the nucleotide sequence of SEQ ID NO:3, a degenerate variant thereof or a complement thereof.

19. An isolated human pancortin polypeptide encoded by a polynucleotide comprising a nucleotide sequence having at least 95% identity to the nucleotide sequence of SEQ ID NO:5, a degenerate variant thereof or a complement thereof.

20. An isolated human pancortin polypeptide encoded by a polynucleotide comprising a nucleotide sequence of SEQ ID NO:7, a degenerate variant thereof or a complement thereof.

21. The polypeptide according to claims 17, 18, 19 or 20, wherein the pancortin polypeptide binds a pablo polypeptide comprising the amino acid sequence of SEQ ID NO: 9, a variant thereof, or a fragment thereof, wherein binding modulates apoptosis in a neural cell.

22. The polypeptide according to claims 17, 18, 19 or 20, wherein the polypeptide is a fusion polypeptide.

23. An isolated human pancortin polypeptide comprising an amino acid sequence of SEQ ID NO:2, a variant thereof or a fragment thereof.

24. An isolated human pancortin polypeptide comprising an amino acid sequence of SEQ ID NO:4, a variant thereof or a fragment thereof.

25. An isolated human pancortin polypeptide comprising an amino acid sequence of SEQ ID NO:6, a variant thereof or a fragment thereof.

26. An isolated human pancortin polypeptide comprising an amino acid sequence of SEQ ID NO: 8, a variant thereof or a fragment thereof.

27. The polypeptide according to claims 23, 24, 25 or 26, wherein the polypeptide binds a pablo polypeptide comprising the amino acid sequence of SEQ ID NO:9 or a variant thereof, wherein binding modulates apoptosis in a neural cell.

28. The polypeptide according to claims 23, 24, 25 or 26, wherein the polypeptide is a fusion polypeptide.

29. An antibody specific for a pancortin polypeptide comprising the amino acid sequence of SEQ ID NO: 2, SEQ ID NO: 4, SEQ ID NO: 6, SEQ ID NO: 8, a variant thereof or a fragment thereof.

30. The antibody of claim 29, wherein the antibody is selected from the group consisting of monoclonal, polyclonal, chimeric, humanized and single chain.

31. The antibody of claim 30, wherein the antibody is monoclonal.

32. An antibody specific for a pablo-pancortin polypeptide dimer.

33. The antibody of claim 32, wherein the polypeptide dimer comprises a pablo polypeptide comprising the amino acid sequence of SEQ ID NO: 9, a variant thereof, or a fragment thereof and a pancortin polypeptide comprising the amino acid sequence of SEQ ID NO: 2, SEQ ID NO: 4, SEQ ID NO: 6, SEQ ID NO: 8, a variant thereof or a fragment thereof.

34. The antibody of claim 33, wherein the antibody is selected from the group consisting of monoclonal, polyclonal, chimeric, humanized and single chain.

35. The antibody of claim 34, wherein the antibody is monoclonal.

36. An expression vector comprising a polynucleotide comprising the nucleotide sequence of SEQ ID NO: 1, a degenerate variant thereof, a complement thereof or a fragment thereof.

37. The vector of claim 36, wherein the polynucleotide encodes a pancortin polypeptide comprising the amino acid sequence of SEQ ID NO:2, a variant thereof or a fragment thereof.

38. An expression vector comprising a polynucleotide comprising the nucleotide sequence of SEQ ID NO:3, a degenerate variant thereof, a complement thereof or a fragment thereof.

39. The vector of claim 38, wherein the polynucleotide encodes a pancortin polypeptide comprising the amino acid sequence of SEQ ID NO:4, a variant thereof or a fragment thereof.

40. An expression vector comprising a polynucleotide comprising the nucleotide sequence of SEQ ID NO:5, a degenerate variant thereof, a complement thereof or a fragment thereof.

41. The vector of claim 40, wherein the polynucleotide encodes a pancortin polypeptide comprising the amino acid sequence of SEQ ID NO:6, a variant thereof or a fragment thereof.

42. An recombinant expression vector comprising a polynucleotide comprising the nucleotide sequence of SEQ ID NO:7, a degenerate variant thereof, a complement thereof or a fragment thereof.

43. The vector of claim 42, wherein the polynucleotide encodes a pancortin polypeptide comprising the amino acid sequence of SEQ ID NO:8, a variant thereof or a fragment thereof.

44. The vector according to claims 36, 38, 40 or 42, further comprising a polynucleotide encoding a pablo polypeptide comprising the amino acid sequence of SEQ ID NO:9, variant thereof or a fragment thereof.

45. The vector according to claims 36, 38, 40 or 42, wherein the polynucleotide is selected from the group consisting of DNA, genomic DNA, cDNA, RNA and antisense RNA.

46. The vector of claim 45, wherein the polynucleotide is operatively linked to one or more regulatory elements selected from the group consisting of a promoter, an enhancer, a splicing signal, a termination signal, a ribosomal binding signal and a polyadenylation signal.

47. The vector according to claims 36, 38, 40 or 42, wherein the vector DNA is selected from the group consisting of plasmid, episomal, YAC and viral.

48. The vector of claim 47, wherein the vector is plasmid DNA.

49. A genetically engineered host cell, transformed, transfected or infected with the vector of claim 36.

50. A genetically-engineered host cell, transformed, transfected or infected with the vector of claim 38.

51. A genetically engineered host cell, transformed, transfected or infected with the vector of claim 40.

52. A genetically engineered host cell, transformed, transfected or infected with the vector of claim 42.

53. The host cell according to claims 49, 50, 51 or 52, wherein the host cell is selected from the group consisting of a bacterial cell, a fungal cell, an insect cell, a plant cell and an animal cell.

54. The host cell of claim 53, wherein the host cell is bacterial.

55. The host cell according to claims 49, 50, 51 or 52, wherein the vector expresses the polynucleotide to produce the encoded polypeptide, variant or a fragment thereof.

56. A neural cell line stably expressing a pancortin polypeptide comprising the amino acid sequence of SEQ ID NO:2, SEQ ID NO:4, SEQ ID NO:6, SEQ ID NO:8, a variant thereof or a fragment thereof.

57. A method for modulating apoptosis in a cell comprising modulating the activity of a pancortin polypeptide.

58. The method of claim 57, further comprising modulating the activity of a pablo polypeptide.

59. A method for modulating apoptosis in a cell comprising modulating the expression of a polynucleotide encoding a pancortin polypeptide.

60. The method of claim 59, further comprising modulating the expression of a polynucleotide encoding a pablo polypeptide.

61. A method for treating a subject for a nervous system disorder comprising modulating the activity of a pancortin polypeptide and/or modulating the expression of a polynucleotide encoding a pancortin polypeptide.

62. A method for assaying the effects of test compounds on the activity of a pancortin polypeptide comprising the steps of:
(a) providing a transgenic animal comprising a polynucleotide encoding a pancortin polypeptide;
(b) administering a test compound to the animal; and
(c) determining the effects of the test compound on the activity of the pancortin in the presence and absence of the test compound.

63. The method of claim 62, wherein the polynucleotide has at least one mutation selected from the group consisting of nucleotide deletion, nucleotide substitution and nucleotide insertion.

64. A method for assaying the effects of test compounds on an animal with a genome comprising a functional disruption of a polynucleotide encoding a pancortin polypeptide, the method comprising:
(a) providing a transgenic animal whose genome comprises a disruption of the endogenous polynucleotide encoding a pancortin polypeptide;
(b) administering a test compound to the animal; and
(c) determining the effects of the test compound on the activity of the pancortin polypeptide in the presence and absence of the test compound.

65. A method for assaying the effects of test compounds on the activity of a pancortin polypeptide comprising the steps of:
(a) providing recombinant cells comprising a polynucleotide expressing a pancortin polypeptide;
(b) contacting the cells with a test compound; and
(c) determining the effects of the test compound on the activity of the pancortin in the presence and absence of the test compound.

66. The method of claim 65, wherein the polynucleotide has at least one mutation selected from the group consisting of nucleotide deletion, nucleotide substitution and nucleotide insertion.

67. The method of claim 66, wherein the cell further comprise a polynucleotide expressing a pablo polypeptide.

68. A method for assaying the effects of test compounds on the binding interaction of pancortin and pablo polypeptides comprising the steps of:
(a) providing yeast cells for a yeast two-hybrid system comprising a pancortin polypeptide and a pablo polypeptide;
(b) contacting the cells with a test compound; and
(c) determining the effect of the test compound on the binding interaction of the pancortin and pablo polypeptides in the presence and absence of the test compound.

69. A method of producing a pancortin polypeptide comprising an amino acid sequence of SEQ ID NO:2, SEQ ID NO:4, SEQ ID NO:6, SEQ ID NO:8, a variant thereof or a fragment thereof, comprising:
(a) transfecting, transforming or infecting a recombinant host cell with an expression vector comprising a polynucleotide comprising a nucleotide sequence of SEQ ID NO:1, SEQ ID NO:3, SEQ ID NO:5, SEQ ID NO:7, or a degenerate variant thereof;
(b) culturing the host cell under conditions sufficient for the production of the polypeptide; and
(c) isolating the polypeptide from the culture.

70. A method for the treatment of a subject in need of reduced pancortin activity comprising:
(a) administering to the subject a therapeutically effective amount of a pancortin antagonist; and/or
(b) administering to the subject a polynucleotide encoding an antisense RNA polynucleotide comprising a nucleotide sequence that is a complement to a nucleotide sequence of SEQ ID NO:1, SEQ ID NO:3, SEQ ID NO:5, SEQ ID NO:7, a degenerate variant thereof or a fragment thereof.

71. A method for the diagnosis of a disease or the susceptibility to a disease in a subject related to the expression or activity of a pancortin polypeptide in the subject comprising:
(a) determining the presence or absence of a mutation in a polynucleotide encoding a pancortin polypeptide comprising an amino acid sequence of SEQ ID NO:2, SEQ ID NO:4, SEQ ID NO:6, SEQ ID NO:8, or a fragment thereof; and/or
(b) assaying for the presence of pancortin expression in a sample derived from the subject, wherein the pancortin expressed is a polynucleotide encoding a pancortin polypeptide comprising an amino acid sequence of SEQ ID NO:2, SEQ ID NO:4, SEQ ID NO:6, SEQ ID NO:8, or a fragment thereof.

72. A composition for treating a hyperproliferative disease comprising a pancortin polypeptide comprising the amino acid sequence of SEQ ID NO:2, SEQ ID NO:4, SEQ ID NO:6, or SEQ ID NO:8 and a pablo polypeptide comprising the amino acid sequence of SEQ ID NO:10.

73. The hyperproliferative disease of claim 72, wherein the disease is selected from the group consisting of cancer, psoriasis, restenosis, atherosclerosis and fibrosis.

74. A nucleic acid molecule which is antisense to a pancortin mRNA molecule.

75. A method of inhibiting expression of a pancortin gene in a cell comprising providing said cell with an antisense nucleic acid.

76. A non-human transgenic mammal whose genome comprises an exogenous polynucleotide which encodes a pancortin polypeptide or a fragment thereof, wherein the polynucleotide expression is under the control of a regulated promoter.

77. The mammal of claim 76, wherein the polynucleotide comprises a nucleic acid sequence of SEQ ID NO:1, SEQ ID NO:3, SEQ ID NO:5 or SEQ ID NO:7.

78 The mammal of claim 77, wherein the polypeptide comprises an amino acid sequence of SEQ ID NO:2, SEQ ID NO:4, SEQ ID NO:6 or SEQ ID NO:8.

79. The mammal of claim 76, wherein the mammal is Rattus norvegicus or Mus musculus.

80. The mammal of claim 76, wherein the regulated promoter is an inducible promoter.

81. The mammal of claim 80, wherein the inducible promoter is Gal4-E1A or tetracycline responsive element (TRE).

82. The mammal of claim 76, wherein the regulated promoter is a tissue specific promoter.

83. The mammal of claim 82, wherein the tissue specific promoter is a neuron specific promoter.

84. The mammal of claim 83, wherein the promoter is mouse Thy 1.2.

85. The mammal of claim 76, wherein the mammal is characterized by a phenotype selected from the group consisting of hind limb tremor, reduced body size, reduced hind limb grasp strength, front limb clasping, hind limb clasping and death.

86. A non-human transgenic mammal whose genome comprises a homozygous disruption in its endogenous pancortin gene, wherein the disruption prevents the expression of a functional pancortin polypeptide.

87. The mammal of claim 86, wherein the mammal is Mus musculus.

88. The mammal of claim 86, wherein the mammal is characterized by a phenotype selected from the group consisting of hind limb tremor, reduced body size, reduced hind limb grasp strength, front limb clasping, hind limb clasping and death.

89. A method for producing a non-human transgenic mammal whose genome comprises an exogenous polynucleotide which encodes a pancortin polypeptide or a fragment thereof comprising the steps of:
(a) introducing into the pronucleus of a fertilized oocyte a polynucleotide comprising a nucleic acid sequence of SEQ ID NO:1, SEQ ID NO:3, SEQ ID NO:5 or SEQ ID NO:7, wherein the polynucleotide is operatively linked to a promoter;
(b) implanting the oocyte into a pseudopregnant non-human mammal, wherein the oocyte develops into an embryo; and
(c) allowing the embryo to develop into a viable transgenic mammal.

90. The method of claim 89, wherein the polynucleotide of SEQ ID NO:1, SEQ ID NO:3, SEQ ID NO:5 or SEQ ID NO:7 encodes a full length pancortin polypeptide having an amino acid sequence of SEQ ID NO:2, SEQ ID NO:4, SEQ ID NO:6 or SEQ ID NO:8.

91. The method of claim 90, wherein the polynucleotide of SEQ ID NO:1, SEQ ID NO:3, SEQ ID NO:5 or SEQ ID NO:7 encodes a mutated Pablo polypeptide.

92. The method of claim 90, wherein the polynucleotide expression is under the control of a constitutive promoter.

93. The method of claim 89, wherein the mammal is characterized by a phenotype selected from the group consisting of hind limb tremor, reduced body size, reduced hind limb grasp strength, front limb clasping, hind limb clasping and death.

94. A method for producing a non-human transgenic mammal whose genome comprises a disruption in its endogenous pancortin gene, the method comprising:
(a) providing a polynucleotide encoding a pancortin polypeptide having a functional disruption;
(b) introducing the disrupted polynucleotide into embryonic stem cells;
(c) selecting those embryonic stem cells that comprise the disrupted polynucleotide;
(d) introducing an embryonic stem cell of step (c) into a blastocyst;
(e) transferring the blastocyst of step (d) to a pseudopregnant animal; and
(f) allowing the transferred blastocyst to develop into a mammal chimeric for the disruption;
wherein the disruption prevents the expression of a functional pancortin polypeptide.

95. The method of claim 94, further comprising breeding the chimeric mammal with a wild-type animal to obtain mammals heterozygous for the disruption.

96. The method of claim 94, further comprising breeding the heterozygous mammal to generate a mammal homozygous for the disruption.

97. The method of claim 94, wherein the mammal is characterized by a phenotype selected from the group consisting of hind limb tremor, reduced body size, reduced hind limb grasp strength, front limb clasping, hind limb clasping and death.
